(19) United States
(12) Patent Application Publication
(10) Pub. No.: US 2008/0288176 A1
(43) Pub. Date: Nov. 20, 2008

(54) Development of Prodrugs Possessing a Nitric Oxide Donor Diazen-1-Ium-1,2-Diolate Noiety Using in Vitro/in Silico Predictions

(76) Inventors: Yun Kau Tam, Hong Kong (CN); Christopher Mark Diaper, Edmonton (CA); Hugh A. Semple, Edmonton (CA); Douglas Thacher Ridgway, Edmonton (CA); Yi-Chan James Lin, Edmonton (CA); Brian Duff Sloley, Edmonton (CA)

Correspondence Address: LAW OFFICES OF ALBERT WAI-KIT CHAN, PLLC, WORLD PLAZA, SUITE 604, 141-07 20TH AVENUE, WHITESTONE, NY, 11357, US

(21) Appl. No.: 12/089,816

(22) PCT Filed: Oct. 13, 2006
(86) PCT No.: PCT/US2006/040382

§ 371 Date: Jun. 23, 2008

Related U.S. Application Data
(60) Provisional application No. 60/726,530, filed on Oct. 13, 2005.

(51) Int. Cl. C07C 245/24; C07D 207/16; C07C 215/12; G06F 19/00

(52) U.S. Cl. 702/19; 534/556; 548/535; 562/568; 534/557

(57) ABSTRACT

The present invention provides a method of using a physiologically-based pharmacokinetic model to select a prodrug molecule (NO—X) comprising a therapeutic agent X (e.g. nonsteroidal anti-inflammatory drug, (NSAID)) and an appropriate nitric oxide donor NO. The NSAID can be a non-selective or selective cyclooxygenase inhibitor or other biocompatible compound comprising a carboxyl group. The pharmacokinetic model uses in vitro and/or in silico data to estimate an optimal set of parameters that can predict whether a particular NO—X candidate is capable of producing desirable therapeutic effects, e.g. enhanced anti-inflammatory activity, reduced intestinal, cardiac and renal toxicity. Accordingly, the present invention can greatly enhance proper selection of an appropriate candidate for drug development, thereby minimizing development time and conserving costs.

### CROSS-REFERENCE TO RELATED APPLICATIONS

This application claims the priority of U.S. provisional applications Nos. 60/726,530, filed Oct. 13, 2005, 60/730,120, filed Oct. 21, 2005, 60/756,446, filed Jan. 5, 2006, and 60/812,230, filed Jun. 9, 2006. The disclosure of the preceding applications are hereby incorporated in their entireties by reference into this application.

### FIELD OF THE INVENTION

This invention relates to development of prodrug molecules comprising a therapeutic agent (such as nonsteroidal anti-inflammatory drug) and a nitric oxide donor.

### BACKGROUND OF THE INVENTION

Since the introduction of aspirin over a hundred years ago, nonsteroidal anti-inflammatory drugs (NSAIDs) have been used to treat patients who suffer from various forms of arthritis. The anti-inflammatory effects of NSAIDs are mainly mediated via the inhibition of cyclooxygenase (COX)-derived prostaglandin (PG) synthesis (Brooks et al., 1991; Cathella-Lawson et al., 2001; Rodriguez et al., 2001; Vane et al., 1998). PG inhibition is also a major side effect of NSAIDs, gastric ulcers (Wallace 2003). The reason is that PG is responsible for vascular homeostasis and gastrointestinal tract protection (Hollander, 1994; Rainsford, 1999; Schoen & Vender, 1989).

In the early 1990s, it was discovered that COX enzyme exists in two isoforms, COX-1 and COX-2. Initially, COX-1 was thought to be a constitutive and a ubiquitous enzyme that is present in a number of tissues including the GI tract (Lipsky, 1999; Buttar and Wang, 2000), whereas COX-2 was regarded as strictly an inducible enzyme. Pro-inflammatory mediators such as cytokines, growth factors, lipopolysaccharides or prostanoids would up-regulate this isoform (Hinz et al., 2000; Hinz and Brune, 2002). In theory, an inhibition of COX-2 isozyme would produce anti-inflammatory effects while sparing the GI from damages. This discovery has led to the development of selective COX-2 inhibitors. At the end of 1999, the first COX-2 inhibitor, CELECOXIB, was introduced to the market. Shortly after the introduction of CELECOXIB, a more specific COX-2 inhibitor, ROFECOXIB, was launched. Clinical studies showed that these COX-2 inhibitors are as effective in fighting pain and inflammation as the other non-selective NSAIDs (Morrison et al., 1999; Reicin et al., 2001; Cannon et al., 2000; Day et al., 2000). In a clinical trial, VIGOR (Bresalier et al., 2005), involving 8,076 patients suffering from rheumatoid arthritis, it was found that ROFECOXIB has significantly less GI side effects when compared to NAPROXEN. In another large clinical trial, CLASS (Silverstein et al., 2000), the superiority of COX-2 inhibitor over non-selective NSAID in the area of GI side effects was demonstrated by comparing CELECOXIB with a non-selective NSAID, DICLOFENAC. The perceived edge of COX-2 inhibitors over non-selective NSAIDs has led to its huge success in the market place. In 2003, CELECOXIB and ROFECOXIB took 75% of the US NSAID market (FitzGerald, 2003). Since then, there has been a race to develop more selective COX-2 inhibitors with better pharmacokinetic characteristics. Examples are VALDECOXIB (or its prodrug PARECOXIB for parenteral use), ETORICOXIB and LUMIRACOXIB.

In a clinical trial, APPROVe, conducted by Merck, investigating the effects of ROFECOXIB in preventing colorectal adenoma, it was discovered that ROFECOXIB, a selective COX-2 inhibitor, was associated with cardiovascular events such as myocardial infarction and cerebral ischemia after 18 months of use. The results of this clinical trial were recently published in the New England Journal of Medicine (Bresalier et al., 2005). The discovery of these potential side effects of ROFECOXIB has led to its withdrawal from the market. Subsequently, the other two COX-2 inhibitors have come under close scrutiny. Basically, it is questioned whether the cardiovascular events are limited to ROFECOXIB or is it a class effect. On Apr. 7, 2005, the Food and Drug Administration (FDA) requested that Pfizer suspend sales of BEXTRA (VALDECOXIB) in the United States. FDA now requires all prescription NSAIDs to provide additional information concerning cardiovascular and gastrointestinal risks.

In order to appreciate FDA's decision on warning labels for all selective and non-selective COX inhibitors, an understanding of arachidonic acid metabolism is necessary (see the following diagram: an arachidonic acid cascade. CYP: cytochrome P₄₅₀ isozymes; EETs: epoxyeicosatrienoic acids; HETEs: hydroxyeicosatetraenoic acids; HODEs: hydroxyoctadecadienoic acids; DP: prostaglandin D₂ receptor; EP: prostaglandin E₂ receptor; IP: prostacyclin receptor; FP: prostaglandin F₂ receptor; TP: prostaglandin T₂ receptor.)

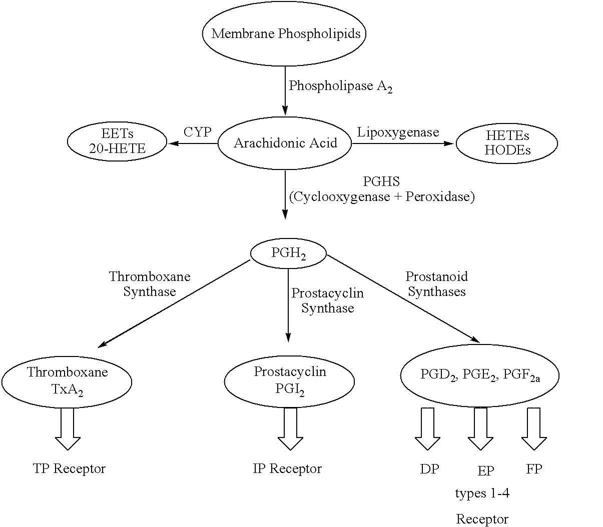

Arachidonic acid (AA) is a metabolic product of membrane-bound phospholipids through phospholipase A₂. AA is a substrate of cyclooxygenase and peroxidase to generate an endoperoxide, prostaglandin H₂ (PGH₂) (Warner and Mitchell, 2004; Davidge 2001; FitzGerald 2003a). PGH₂ is the precursor of thromboxane A₂ (TxA₂), prostanoids and prostacyclins. These reactions are mediated through tissue specific enzymes thromboxane synthase, prostanoid synthase and prostacyclin synthase, respectively. Inhibition of COX-1 isoform will lead to a reduction in the circulatory thromboxane and prostaglandin levels. In addition to other tissues, COX-1 is found in the platelet and the GI tract. Since platelet aggregation is atherogenic, a reduction of thromboxane in blood would reduce the risk of thrombi formation and therefore cardiovascular ischemia (Krötz et al., 2005). On the other hand, inhibition of COX-2 isozyme will lead to a reduction of PGI₂. This prostacyclin is a potent vasodilator and an anti-platelet agent (Krötz et al., 2005). Inhibition of COX-2 isozyme has been postulated to cause the untoward cardiovascular events that have been observed in patients (Krötz et al., 2005). Non-selective COX inhibitors such as the traditional NSAIDs (e.g. ASPIRIN, IBUPROFEN, NAPROXEN, INDOMETHACIN, etc.) have various degrees of COX-1 and 2 inhibition; therefore, they may have various degree of cardiovascular risks.

In a review written by Krötz et al. (2005), it was postulated that the balance between thromboxane and PGI₂ is a crucial determinant of cardiovascular events for both selective and non-selective NSAIDs. This has led to the postulation that the selectivity of COX inhibition has an important role to play in the cardiovascular safety of NSAIDs. Besides the ratio of COX-1/COX-2 selectivity, pharmacokinetic properties, dosage and the cardiovascular state of patients are also factors that can contribute to the observed cardiovascular risks.

Contrary to previous beliefs, the COX-2 isozyme is not only inducible, it is also expressed constitutively (Zimmermann et al., 1998; Iseki 1995; Nantel et al., 1999; Chakraborty et al., 1996; Slater et al., 1999, 1999a; Damm et al., 2001; Tegeder et al., 2000). This enzyme is expressed in various tissues including gut (Zimmermann et al., 1998; Iseki 1995), myometrium (Slater et al., 1999, 1999a) and kidneys (Tegeder et al., 2000). COX-2 inhibition can lead to sodium and fluid retention which may lead to hypertension (Krötz, 2005). Hypertension is a known atherogenic factor.

Nitric oxide (NO) is now widely recognized as a critical mediator of gastrointestinal mucosal defense, exerting many of the same actions as prostaglandins in the gastrointestinal tract (Wallace 2003). NO has been shown to reduce the severity of gastric injury in experimental models (McNaughton et al., 1989; Kitagawa et al., 1990). It has been proposed that linking a NO-releasing moiety to a NSAID may reduce the toxicity of the latter (Wallace et al. 1994). In animal studies, NO-releasing derivatives of a wide range of NSAIDs (FIG. 1), including NO-aspirin, NO-naproxen, NO-flurbiprofen, and NO-diclofenac, have been shown to spare the gastrointestinal tract, even though they suppressed prostaglandin synthesis as effectively as the parent drug (Wallace et al., 1994a and 1994b; Reuter et al., 1994; Cuzzolin et al., 1995; Davies 1997). A number of other NO-NSAIDS have been disclosed which utilize nitrooxyalkyl functionality as the source of NO (Ranatunge et al., 2006; Kartasasmita et al., 2002; Andersson et al., 2004; Gilmer et al., 2002; Almirante et al., 2006; Benedini et al., 2000; Bolla et al., 2005; Rivolta et al., 2005; Del Soldato 2002a, 2002b, 2003, 2004a and 2004b; Bandarage et al., 2000; Earl et al., 2004; Satyam 2006).

However, an important drawback to this design is that production of NO from organic nitrate esters has been reported to occur via a number of mechanisms, both enzymatic (glutathione S-transferase, cytochrome P-450 and other uncharacterized enzymes) and chemical (non-proteinous thiols) (Fung 2004). Organic nitrate esters also demonstrate a reduction of efficiency on continued use of the drugs, contributing to “nitrate tolerance” (Const and Ferdinandy 2005). In this regard, N-diazen-1-ium-1,2-diolates (also referred to as diazeniumdiolates or NONOates) have the potential to release up to 2 equivalents of NO with half-lives that correlate well with their pharmacological durations of action. These observations suggest that O²-unsubstituted diazeniumdiolates are minimally affected by metabolism, and are essentially different from currently available clinical vasodilators (Keefer 2003).

O²-Substituted diazeniumdiolates possess three attributes that make them especially attractive for designing drugs to treat a variety of disease states, namely structural diversity, dependable rates of NO release, and rich derivatization chemistry that facilitates targeting of NO to specific target organ and/or tissue sites (Keefer 2003). Unsubstituted diazeniumdiolates may be derivatized at the O² position to form NO donors which are much more resistant to physiological conditions, resulting in a pronounced increase in the half life of the NO donor. Saavedra et al. (1999) reported a NO-NSAID based on an O²-methoxy substituted diazeniumdiolate derived from piperazine. The NSAID IBUPROFEN was covalently attached to the distal nitrogen of the piperazine linker via an amide bond. The O²-methoxy diazeniumdiolate spontaneously released NO under physiological conditions with a half life of approximately 17 days. Alternatively, diazeniumdiolates can be substituted at the O²-position with acetyloxy methyl functionality which is resistant to physiological conditions but susceptible towards enzymatic hydrolysis on exposure to esterases (Saavedra et al. 2000). These NO generating moieties can be linked to other biocompatible compounds such as NSAIDS so that the NSAID and unsubstituted diazeniumdiolate are enzymatically released. Knaus et al. (2005) disclosed a series of novel NSAID molecules of this type possessing diazeniumdiolates as NO donors. These molecules have been shown to have excellent gastric protective effects in rats. However, the profiles of NO-NSAID and its active metabolites, NO donor and NSAID, absorption and disposition have not been elucidated. Furthermore, the effects of these candidates on kidney and cardiovascular function are not known.

In light of the COX-2 inhibitor debacle, there is an interest in developing an NO-NSAID using COX-2 inhibitors. It has been shown that NO has beneficial effects on the cardiovascular system and the kidneys (Mollace et al., 2005). It would be logical to synthesize and/or to administer a COX-2 inhibitor with a NO donor. Connor and Manning (2005) described a method comprising the administration of a combination of a COX-2 inhibitor and a nitric oxide donating agent.

Unlike traditional non-selective COX inhibitors which possess a carboxyl group for forming a covalent linkage with a nitric oxide donor such as diazeniumdiolates, COX-2 inhibitors like ROFECOXIB do not always have a functional handle that would readily allow the attachment of a nitric oxide donor moiety. However, it has been reported that certain COX-2 inhibitors, and prodrugs of specific COX-2 inhibitors (for example ROFECOXIB) do contain carboxylic acids that can be covalently bound to a nitrooxyalkyl NO donor via an ester linkage (Engelhardt et al., 2006; Del Soldato et al., 2004c; Letts et al., 2003). Some COX-2 inhibitors such as CELECOXIB and VALDECOXIB contain sulfonamide functionality that has been used as a site of covalent linkage to a nitrooxyalkyl NO donor (Del Soldato et al., 2004c; Bandarage et al., 2004). Alternate strategies for attaching NO donors to COX-2 inhibitors include pyrazoles containing a nitrate ester (ONO₂) moiety as a nitric oxide (NO)-donor (Ranatunge et al., 2004; Khanapure et al., 2002). Bandarage et al. (2003) formed nitrosated and nitrosylated COX-2 inhibitors through one or more sites such as oxygen (hydroxyl condensation), sulfur (sulfhydryl condensation) and/or nitrogen. Dhawan et al. (2005) studied the pharmacology of a nitrated VALDECOXIB derivative, 4-{5-[(nitrooxy)methyl]-3-phenylisoxazol-4-yl}benzenesulfonamide. Khanapure et al. (2004) have synthesized a series of nitric oxide derivatives of COX-2 inhibitors. A series of O²-unsubstituted N-diazeniumdiolate salts was reported to be attached to the COX-2 inhibitor through an aryl nitrogen. The absence of substituent at O² suggests that the nitric oxide release from the diazeniumdiolate derivative would follow that of other reported O²-unsubstituted N-diazeniumdiolates. Therefore, it is assumed that these derivatives release NO directly and do not require the action of enzymes in vivo for this to occur. This type of derivative may also lack tissue specificity in terms of NO donor and NO release. The O²-substituted diazeniumdiolates synthesized by Knaus et al. (2005), on the other hand, require the action of esterases to release the NO donor and subsequently, NO. Tissue specific delivery of NO, to some extent, can be accomplished by adjusting the molecular structure to achieve a desired hydrolysis rate in various organs such as the GI tract, liver, blood, etc. However, the adjustment of the hydrolysis rate has not been taken into consideration as the pharmacokinetics of these moieties is unknown.

The NO-donating diazeniumdiolate NO-NSAIDs described by Knaus, et al. (2005) are designed to be released in blood by serum esterases. This approach of NO-NSAID design may not be optimal. Esterases are traditionally known to be non-specific. However, recent studies show that there are higher concentrations of certain esterases in specific organs such as liver and intestine. It has been shown that exposure of orally administered NO-NSAIDs, which include the ones synthesized by Knaus et al. (2005), NCX-4016 and AZD3582, to plasma is minimal. Hence, in the design of NO-NSAIDs, a systematic approach which takes into account the drug-like properties of these candidates is imperative. A flexible molecular library of O²-substituted diazeniumdiolate NO-NSAID candidates is required to generate and modify their properties in a controlled fashion.

### SUMMARY OF THE INVENTION

The present invention provides a physiologically based pharmacokinetic/pharmacodynamic model. This model requires in vitro/in silico input to estimate pharmacokinetic/pharmacodynamic parameters of a test candidate. This model is useful for: (1) screening a NO-NSAID candidate for its suitability of development, and (2) providing information for synthesis of a new NO-NSAID (both selective and non-selective) candidate that may have a better chance of success in the development process.

The physiologically based pharmacokinetic/pharmacodynamic model of the present invention contains a series of compartments that describe the time course of a nonsteroidal anti-inflammatory prodrug, its active metabolites and nitric oxide release in intestine, liver, kidneys, blood/plasma and heart after prodrug administration. The time course of the prodrug, its active metabolites and nitric oxide release can be simulated using a series of in vitro and in silico inputs. The stability of each component in the gastrointestinal lumen is estimated using data collected from artificial gastric and intestinal juice. Intestinal metabolism is estimated using intestinal microsomes and absorption rate is estimated using permeability data collected from a cell monolayer such as Caco-2. Hepatic elimination is estimated using liver microsomes and stability in plasma is calculated using degradation of each component in the media. Plasma protein binding can either be measured using a standardized in vitro method or it can be estimated using an in silico method. The distribution of each component in various parts of the body is estimated using an in silico method. The rate of nitric oxide release is estimated using an in vitro endothelial cell model. The time course of prodrug, its active metabolites and nitric oxide can be simulated in human and animal using this physiologically based pharmacokinetic/pharmacodynamic model provided that the corresponding in vitro and in silico data are used as inputs.

This model has been used successfully to predict the time course of NO-NSAID prodrugs and NSAID after prodrug administration. Advantages and deficiencies of existing NO-NSAIDs were identified. Based on these results, a general structure of an NO-NSAID which would provide an optimal delivery of nitric oxide to the gut, heart and kidneys has been designed. This NO-NSAID molecule contains an NSAID molecule which is connected to a nontoxic linker (e.g. an amino acid) through an alkyl diester. A nitric oxide donor is attached to the linker through an ester bond on the other end. The nitric oxide releasing moiety is preferably a diazeniumdiolate

NSAID applicable in the present invention includes, but is not limited to, non-selective COX inhibitors such as acetylsalicylic acid (ASA, CH₃COOC₆H₄COOH), IBUPROFEN (C₁₃H₁₈O₂), naproxen (NAP, C₁₄H₁₄O₃,), indomethacin (C₁₉H₁₆ClNO₄), or diclofenac (C₁₄H₁₀Cl₂NNaO); selective COX-2 inhibitors such as CELECOXIB which contain a sulfonamide group or prodrugs of ROFECOXIB which contains a carboxyl group.

In one embodiment, the present invention provides a method of pairing a therapeutic agent with an appropriate nitric oxide donor to create an effective prodrug molecule. The method comprises: (i) obtaining in vitro or in silico pharmacokinetic or pharmacodynamic data, (ii) placing the data into a physiologically-based pharmacokinetic model comprising a compartment model which divides a gastrointestinal tract into compartments, and a second compartment model which divides a body into plasma/blood and tissue compartments, and (iii) generating output parameters from the pharmacokinetic model, wherein the output parameters determine the pairing of a therapeutic agent with an appropriate nitric oxide donor to create an effective prodrug molecule.

The present invention also provides a prodrug molecule selected by the above method, wherein the prodrug molecule comprises a therapeutic agent and a nitric oxide donor.

In another embodiment, there is provided a prodrug molecule comprising a nonsteroidal anti-inflammatory drug and a nitric oxide releasing moiety, wherein the moiety has a half-life that is longer than the total time period for hydrolysis and absorption, and wherein a therapeutic dosage of nitric oxide is released into enterocytes, thereby protecting against damage caused by gastrointestinal irritation, bleeding or ulceration.

The present invention also encompasses uses of the prodrug molecules identified by the method described herein to provide therapeutic treatments. The present invention also provides a kit comprising the prodrug molecules identified or described herein.

The present invention also provides a prodrug molecule comprising: (i) a nitric oxide releasing moiety linked to an amino acid through a linkage that is susceptible to enzymatic hydrolysis or cleavage, and (ii) a therapeutic agent directly linked to said amino acid, or linked to said amino acid through a spacer, wherein the linkage between the therapeutic agent and the spacer, or the linkage between the spacer and the amino acid is susceptible to enzymatic hydrolysis or cleavage, wherein the release of the nitric oxide releasing moiety and the therapeutic agent from the prodrug molecule can be controlled independently.

In another embodiment, there is provided a compound with the formula of:

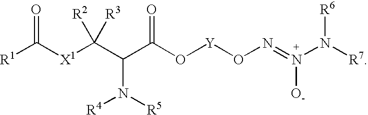

In another embodiment, there is provided a compound with the formula of:

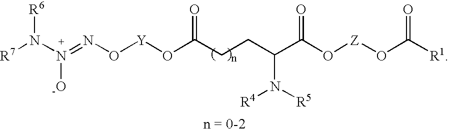

The present invention also provides a compound with the formula of:

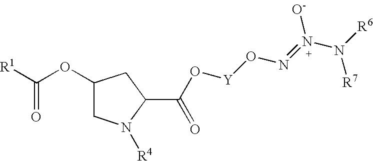

The present invention also provides a compound with the formula of:

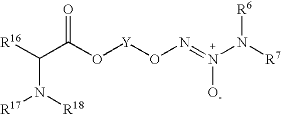

The present invention also provides a compound with the formula of:

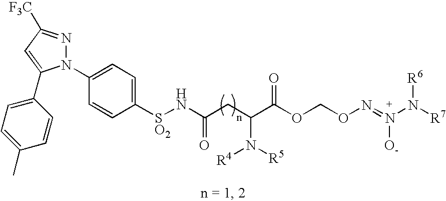

The present invention also provides a compound with the formula of:

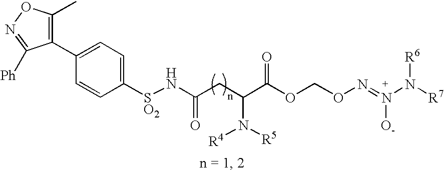

### BRIEF DESCRIPTION OF THE DRAWINGS

FIG. 1 shows the chemical structures of some representative NO-NSAIDS (organic nitrates).

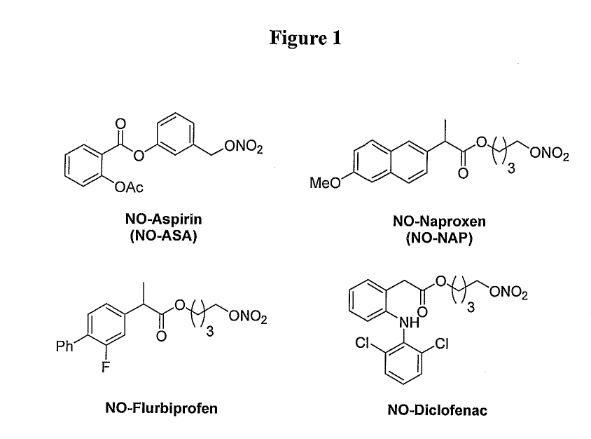

FIG. 2 shows the structures of N3-108 and N3-112.

FIG. 3 shows hydrolysis of PYRO-NO-ASA (N3-108) in human intestinal microsomes.

FIG. 4 shows hydrolysis of PYRO/NO-ASA (N3-108) in human liver microsomes.

FIG. 5 shows hydrolysis of DMA/NO-ASA (N3-112) in human intestinal microsomes.

FIG. 6 shows hydrolysis of DMA/NO-ASA (N3-112) in human liver microsomes.

FIG. 7 shows ulcer index of NAPROXEN, its two diazeniumdiolate prodrug compounds and their comparators.

FIG. 8 shows AUC₀₋₆ₕ (μM-h) of NAPROXEN after dosing with its two diazeniumdiolate prodrug compounds and AZD3582.

FIG. 9 shows serum nitrate concentrations for NAPROXEN, its two diazeniumdiolate prodrug compounds and their comparators.

FIG. 10 shows cardiac tissue prostacyclin (PGI2) to thromboxane A2 (TXB2) ratios for NAPROXEN, its two diazeniumdiolate prodrug compounds and their comparators.

FIG. 11 shows ratios of urine alanine aminopeptidase to creatinine concentration (AAP/Cr) for NAPROXEN, its two diazeniumdiolate prodrug compounds and their comparators.

FIG. 12 shows urine N-acetylglucosaminidase to creatinine concentration ratios (NAG/Cr) for NAPROXEN, its two diazeniumdiolate prodrug compounds and their comparators.

FIG. 13 shows the layout of the entire pharmacokinetic/pharmacodynamic model with simplified organ modules.

FIG. 14 shows the layout of the intestinal segment modules.

FIG. 15 is a detailed layout of intestinal segment 1 showing input from the stomach compartment and blood flow divided between enterocytes and intestinal tissue.

FIG. 16 is a detailed layout of intestinal segment 2 showing blood flow divided between enterocytes and intestinal tissue.

FIG. 17 is a detailed layout of intestinal segment 3 showing blood flow divided between enterocytes and intestinal tissue.

FIG. 18 is a detailed layout of intestinal segment 4 showing blood flow divided between enterocytes and intestinal tissue.

FIG. 19 is a detailed layout of intestinal segment 5 showing blood flow divided between enterocytes and intestinal tissue.

FIG. 20 is a detailed layout of intestinal segment 6 showing blood flow divided between enterocytes and intestinal tissue.

FIG. 21 is a detailed layout of intestinal segment 7 showing blood flow divided between enterocytes and intestinal tissue.

FIG. 22 is a detailed layout of gastric compartment where dose is introduced.

FIG. 23 is a detailed layout of heart compartment.

FIG. 24 is a detailed layout of kidney compartment.

FIG. 25 is a detailed layout of liver compartment showing its dual (portal and arterial) blood supply.

FIG. 26 is a detailed layout of plasma, arterial and venous compartments.

FIG. 27 is a detailed layout of tissue compartment showing bidirectional distribution from the capillary bed into the interstitial fluid and hence to the intracellular space and back.

FIG. 28 is a detailed layout of lung compartment.

FIG. 29 shows a comparison between literature (circles) and simulated results from the pharmacokinetic/pharmacodynamic model for NAPROXEN after administration of 3 mg/kg naproxen in rats. - is a line generated using the model. The shaded area is a two-fold variation of the estimated values. The circles are data obtained from FIG. 1 of Runkel et al. (1972).

FIG. 30 shows a comparison between literature (circles) and simulated results from the pharmacokinetic/pharmacodynamic model for NAPROXEN after administration of 300 mg naproxen in human. - is a line generated using the model. The shaded area is a two-fold variation of the estimated values. The circles are data obtained from FIG. 6 of Runkel et al. (1972).

FIG. 31 is a comparison between literature (circles) and simulated results from the pharmacokinetic/pharmacodynamic model for AZD 3582 and NAPROXEN after administration of 15 μmol/kg AZD 3582 in rats. - is a line generated using the model. The shaded area is a two-fold variation of the estimated values. The circles are data obtained from FIG. 3 of the Fagerholm preclinical paper (2005).

FIG. 32 shows a comparison between literature (circles) and simulated results from the pharmacokinetic/pharmacodynamic model for AZD 3582 and NAPROXEN after administration of 375 mg AZD 3582 in human. - is a line generated using the model. The shaded area is a two-fold variation of the estimated values. The circles are data obtained from FIG. 6 of the Fagerholm clinical paper (2005).

FIG. 33 shows a model estimation of distribution of AZD 3582 in a rat after a 50 mg/kg of AZD 3582 was administered orally.

FIG. 34 shows a model estimation of distribution of AZD 3582 in a 70 kg human after a 50 mg/kg of AZD 3582 was administered orally.

FIG. 35 shows a model estimation of the distribution PYRO/NO-NAP (N-119) in a rat after a 50 mg/kg of N-119 was administered orally.

FIG. 36 shows the Diazenium Diolate candidates reported by Knaus et al., 2005.

FIG. 37 shows a general structure of the new generation NO—NSAID which can be adjusted to provide systemic delivery of NO.

FIG. 38 shows a COX2-AA-NONOate Prodrugs based on Sulfonamide COX-2 Inhibitors.

FIG. 39 shows hydrolysis of di-NAPROXEN prodrug (NAP-AA-NAP).

FIG. 40 shows the structure of DMA/NO-AA-DMA/NO.

FIG. 41 shows differential enzymatic hydrolysis of NO-AA-NSAIDS.

FIG. 42 shows a pathway of hydrolysis for CMD113 and CMD114.

FIG. 43 shows a second pathway of hydrolysis for CMD113 and CMD114.

FIG. 44 shows hydrolysis of CMD113 in rat intestinal microsomes. Naproxen-AA and NO-AA are meant to be trends only, they are not quantitative.

FIG. 45 shows hydrolysis of CMD113 in rat liver microsomes. Naproxen-AA and NO-AA are meant to be trends only, they are not quantitative.

FIG. 46 shows hydrolysis of CMD113 in human intestinal microsomes. Naproxen-AA and NO-AA are meant to be trends only, they are not quantitative.

FIG. 47 shows hydrolysis of CMD113 in human liver microsomes. Naproxen-AA and NO-AA are meant to be trends only, they are not quantitative.

FIG. 48 shows hydrolysis of CMD114 in rat intestinal microsomes. Naproxen-AA and NO-AA are meant to be trends only, they are not quantitative.

FIG. 49 shows hydrolysis of CMD114 in rat liver microsomes. Naproxen-AA and NO-AA are meant to be trends only, they are not quantitative.

FIG. 50 shows hydrolysis of CMD114 in human intestinal microsomes. Naproxen-AA and NO-AA are meant to be trends only, they are not quantitative.

FIG. 51 shows hydrolysis of CMD114 in human liver microsomes. Naproxen-AA and NO-AA are meant to be trends only, they are not quantitative.

FIG. 52 shows a proposed differential hydrolysis of NO-AA-COX2 prodrugs.

FIG. 53 shows a general method for the preparation of nonoate-amino acid-NAPROXEN prodrugs 26.

FIG. 54 shows a general method for the preparation of N-Ac NAP-Glu-NAP 9.

FIG. 55 shows a general method for the preparation of N-Ac DMA/NO-Glu-NO/DMA 14.

FIG. 56 shows a general method for the preparation of CMD113 and CMD114.

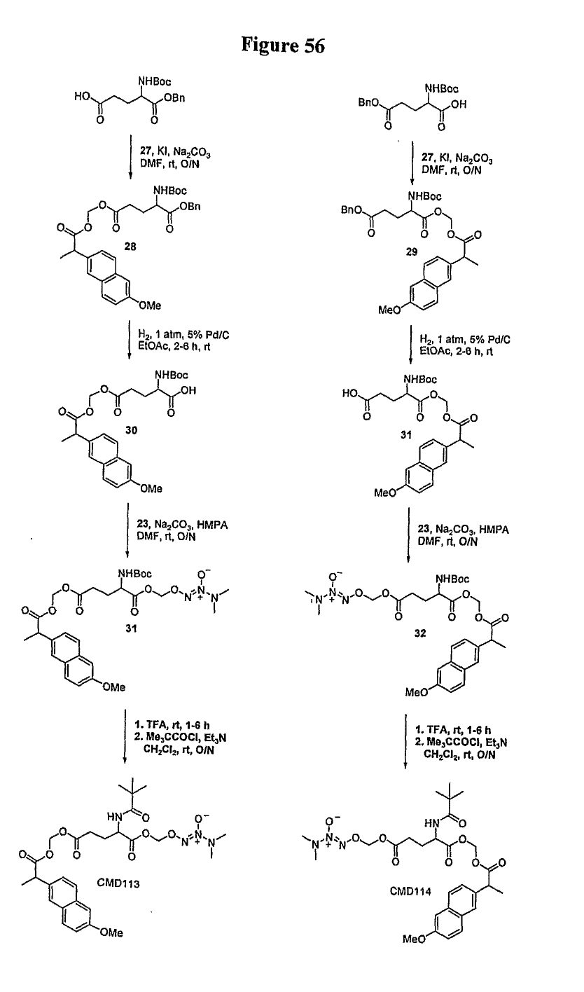

### DETAILED DESCRIPTION OF THE INVENTION

In order to produce a lead with a reasonable chance of success in clinical trials, it is imperative to understand the relative pharmacokinetic and pharmacodynamic of the NSAID, the diazeniumdiolate derivative and the NO donor. This set of information is important for selecting an appropriate NO donor for a NSAID. The present invention provides a process by which a physiologically based pharmacokinetic/pharmacodynamic model requiring in vitro and in silico input is used to predict the pharmacokinetic and pharmacodynamic behaviors of the prodrug moiety.

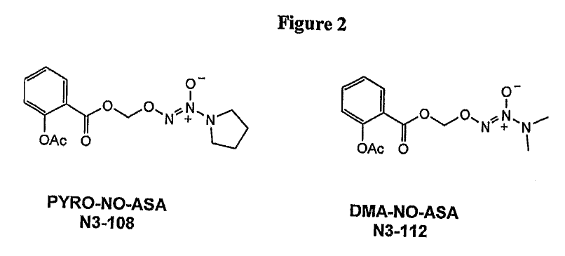

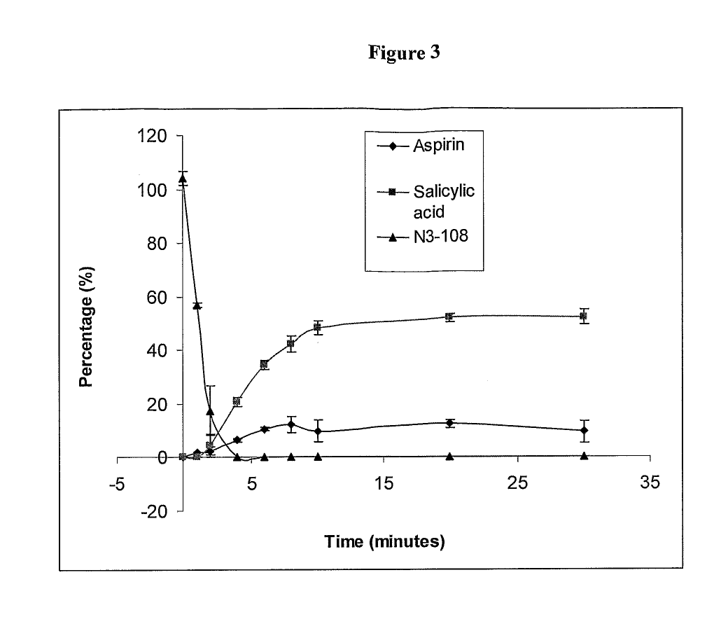

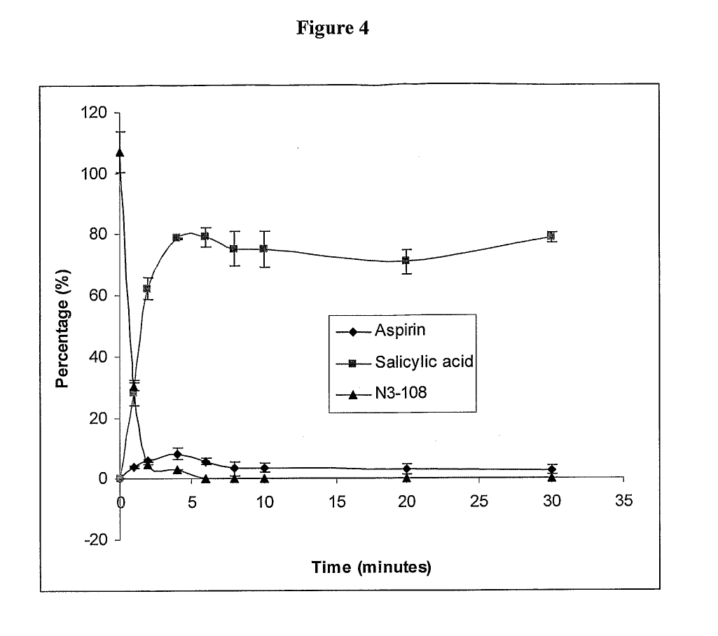

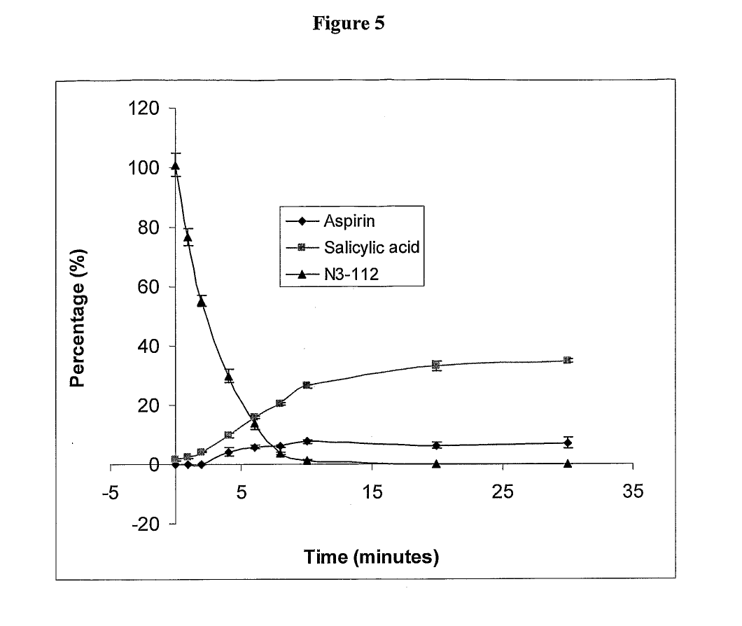

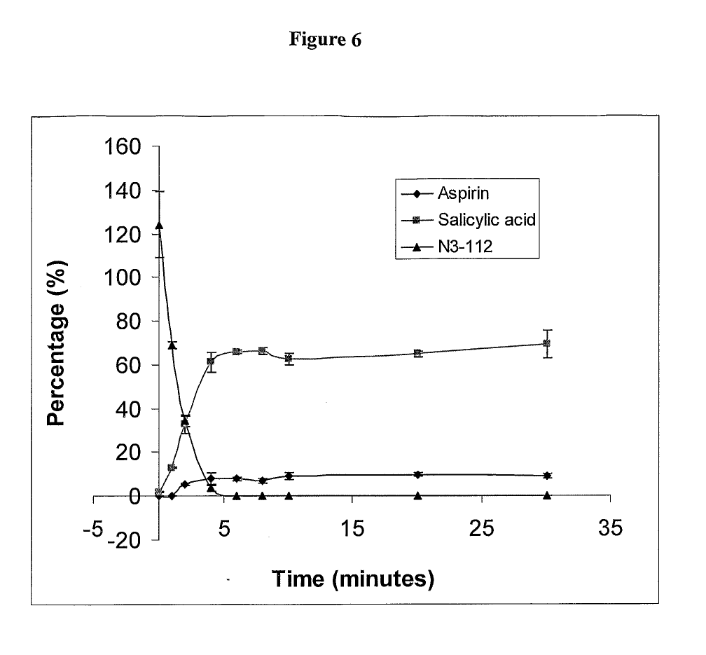

The selection of in vitro tests is designed to provide parameters for the above mentioned model. The use of an inappropriate test would result in wrong predictions. For example, Knaus et al. (2005) used guinea pig serum and porcine esterases to hydrolyze their O²-substituted diazeniumdiolate derivatives of ASPIRIN, as disclosed in U.S. Ser. No. 60/681,842. These tests provided very little information in terms of in vivo human NO-NSAID metabolism rate and the extent to which metabolic conversion to NO donor and NSAID occurred. However, when some of these candidates such as PYRO-NO-ASA (N3-108) and DMA-NO-ASA (N3-112) (FIG. 2) were incubated with human intestinal and liver microsomes (FIGS. 3 to 6), it was found that they are rapidly hydrolyzed. The half-life values were less than one minute. Based on these observations, it has become apparent that prior to entering the systemic circulation, it is likely that the NO-NSAID would have been hydrolyzed in the enterocytes and little or no NO-NSAID would have been detected in the blood stream. Furthermore, the release of NO will commence immediately once the NO donor is detached from the NSAID.

Prodrug design using esterases to release the active principle has been commonly employed (Beaumont et al., 2003). The function and distribution of esterases have been studied extensively, particularly in the last few years. Although esterases are known to be non-specific, there are dramatic inter-species and inter-organ differences. The use of wrong esterases for prodrug development has led to wrong lead selection and therefore, failure (Beaumont et al., 2003; Mizen and Burton 1998).

The release of NO donor in enterocytes provides a higher probability of gastrointestinal protection. However, it is not certain whether NO-NSAID containing diazeniumdiolates would provide protection to other organs such as the heart and kidneys. Gao's group (Frehm et al., 2004) along with other research groups (Singel et al., 2005) have been investigating hemoglobin as a nitric oxide carrier in the blood. This is a hypothesis that describes systemic delivery of nitric oxide to various tissues. In his latest commentary, Gao (2005) stated, “Nitric oxide should never be considered as a solitary and discrete chemical entity in any biological systems.”

Tissue specific delivery may be improved using an appropriate diazeniumdiolate molecule. Keefer and his co-workers have developed a series of diazeniumdiolates with NO generating half-lives ranging from 2 seconds to over 20 hours (Keefer, 2005). Furthermore, Keefer et al. has functionalized diazeniumdiolates with carbohydrates so that the NO is released by the action of glucosidases, thereby limiting NO release to tissues containing this class of enzyme (Showalter et al., (2005). However, in the absence of a systematic approach, the selection of an appropriate diazeniumdiolate is challenging.

The challenge of developing a successful prodrug molecule NO—X (e.g. NO-NSAID) for the treatment of arthritis, cardiovascular and other ailments including cancer is due largely to the difficulty of obtaining the desired rate, extent, and site of nitric oxide release.

For example, if a molecule of a NO-NSAID is predominantly absorbed into the systemic circulation after oral administration, gastric and intestinal membranes can be protected from ulceration only by the nitric oxide released in the blood. The concentration of nitric oxide in the stomach and intestine may not be high enough because NO donor concentration in the blood will be at least an order of magnitude lower than the concentration existing locally in the intestine during the absorption process.

If the NO donor has a short half-life, this will probably not be sufficient to protect the gastrointestinal tract from a NSAID with a longer half-life because the NO released has a very short duration in the body.

Rapid release of NO donor from NO-NSAID in the enterocytes during the absorption process may provide optimal gastrointestinal protection; however, the concentration of nitric oxide in other organs such as heart and kidneys may not be high enough for protection because the NO never reaches the systemic circulation.

The present invention provides a physiologically based pharmacokinetic/pharmacodynamic model for estimating an optimal set of parameters for chemically pairing an NSAID or other therapeutic or biocompatible agents with an appropriate NO donor such as diazeniumdiolate.

The prodrug design approach described herein is not only applicable to NO-NSAID. Other therapeutic or biocompatible agents can be linked to a NO donor such as diazeniumdiolate to optimize delivery and release in specific organs. The use of a biocompatible principle for this purpose is a design for diazeniumdiolate as the sole therapeutic agent.

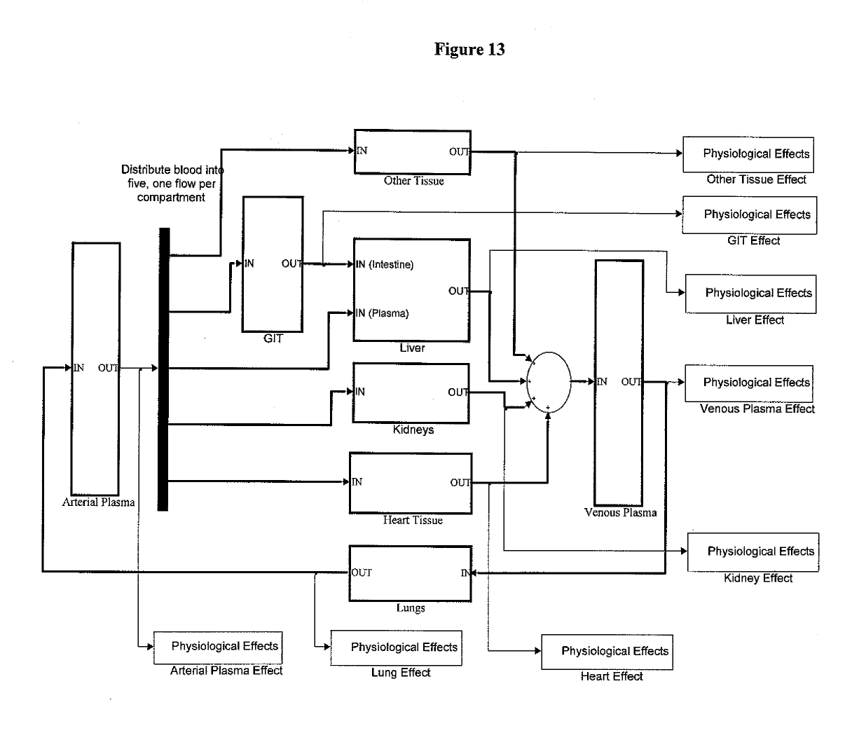

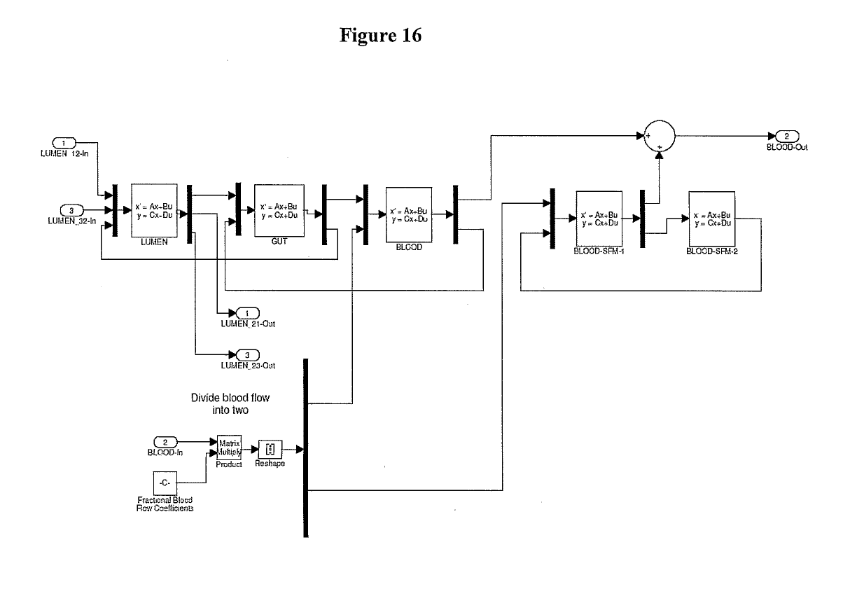

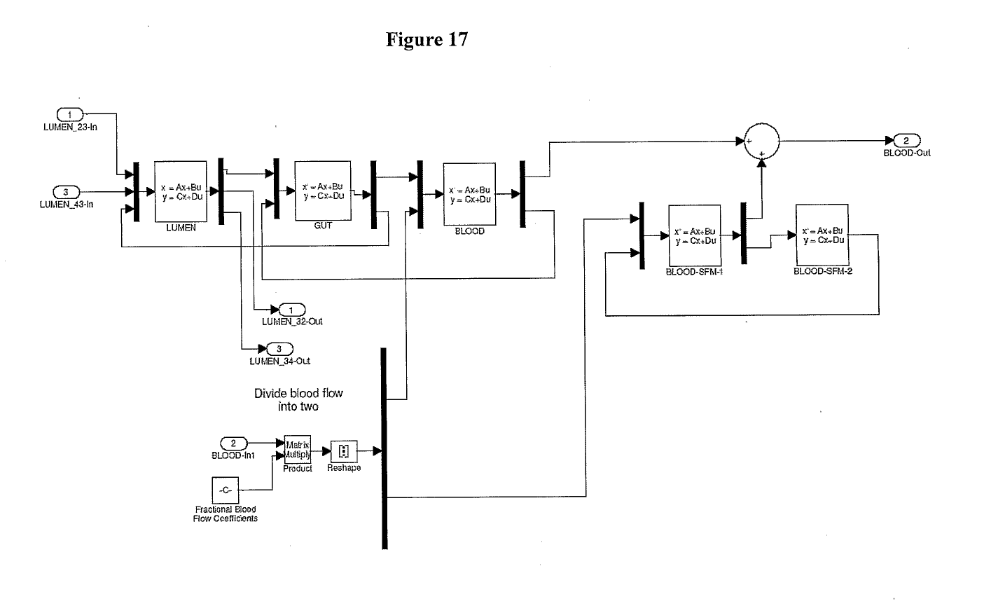

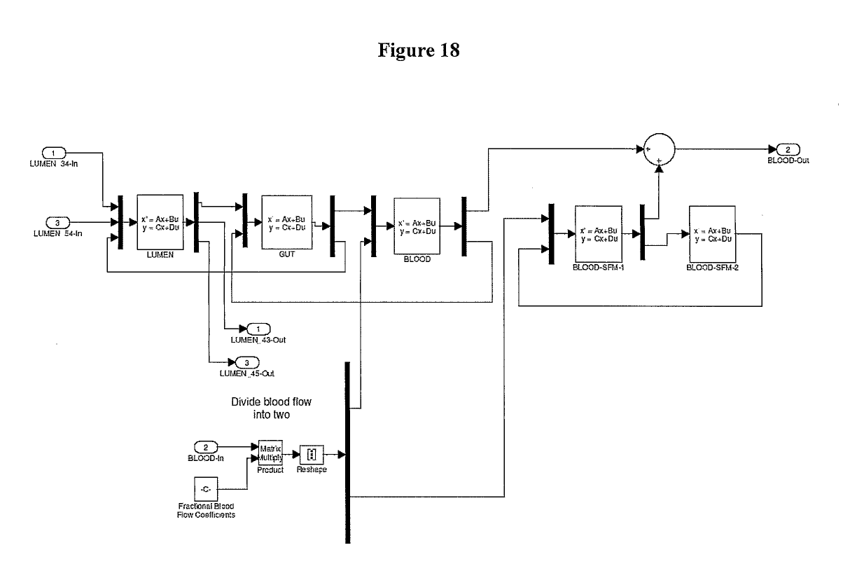

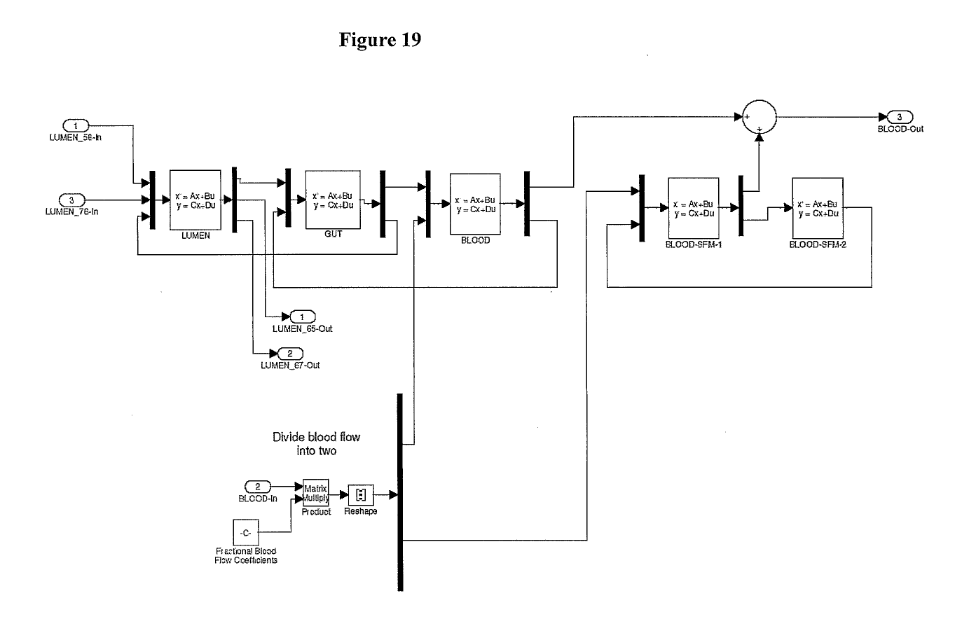

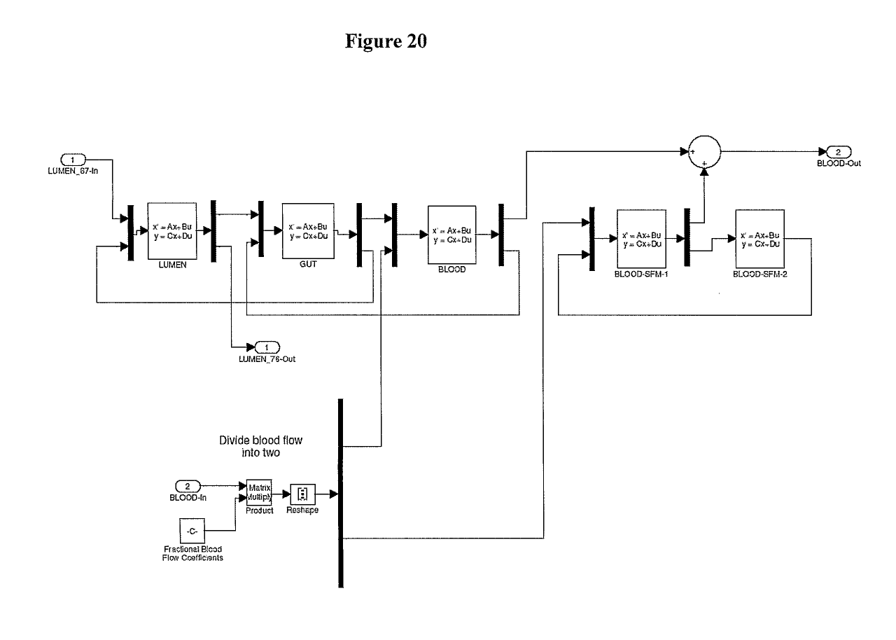

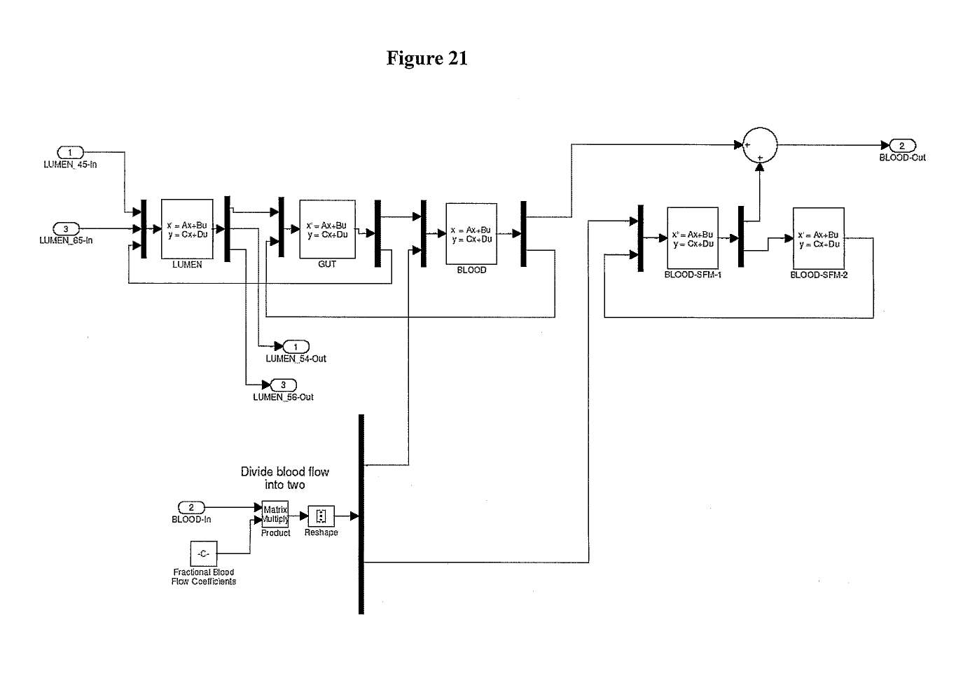

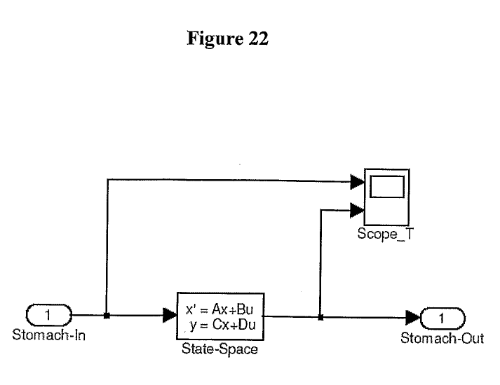

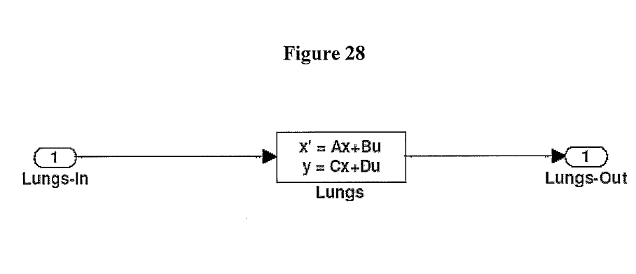

The pharmacokinetic/pharmacodynamic model of the present invention describes the time course of absorption, distribution, metabolism, NO release (FIGS. 13-28), and COX inhibition in animals and human. This pharmacokinetic/pharmacodynamic model comprises a seven compartment model to describe gastrointestinal absorption and a number of physiological compartment models to describe the time course of individual species in the rest of the body including relevant organs and tissue reservoirs. Pharmacodynamic compartments describing the time course of NO release and COX inhibition are attached to the appropriate pharmacokinetic compartments (FIG. 13). The same pharmacokinetic/pharmacodynamic model can be easily adapted to describe the time course of other prodrug moieties. In one embodiment, input parameters of this model are obtained from a series of in vitro tests or in silico estimates of the NO-NSAID or its active and stable metabolite, for example, a molecule that contains a diazeniumdiolate and a linker molecule:

Representative in vitro tests or in silico estimates include: (a) pKa estimation or measurement; (b) Log P measurement or in silico estimation; (c) Solubility in various physiological fluids; (d) Permeability. Caco-2 and/or NOVOKIN's proprietary animal and human cell lines can be used to obtain this parameter; (e) Metabolic rate in the intestine and liver. Human or animal intestinal microsomes, S9 fraction, and cytosol can be used for this purpose. Human or animal hepatocytes, liver microsomes, S9 fraction, and cytosol can also be used; (f) Hydrolysis in human or animal plasma; (g) Serum or plasma protein binding. It can be measured in vitro or estimated in silico; (h) The rate of NO release; (i) Existing pharmacokinetic and pharmacodynamic data of NSAID or a biocompatible agent; (j) Existing NO release rate of known diazeniumdiolate if applicable; (k) Stability in gastric and intestinal environment; and (l) In silico volume of distribution estimation.

Representative outputs of this simulation for a particular NO—X (e.g. NO-NSAID) species are listed as follows: (a) Stability of NO—X in the gastrointestinal tract; (b) Time course and extent of NO—X absorption in the intestine; (c) Time course and extent of NO and X release in the enterocytes; (d) Time course of NO generation from the NO donor in various tissues including gastrointestinal tract, liver, heart and kidneys; (e) Time course of COX-1 inhibition in the intestine; (f) Time course of NO in blood; (g) Time course of NO in tissues including gastrointestinal tract, liver, heart and kidneys; (h) Time course of NO in blood and tissues including gastrointestinal tract, liver, heart and kidneys; (i) Estimation of systemic effect contributed by nitric oxide.

For example, an optimal candidate of NO-naproxen for treating arthritis should have the following parameters: (a) Stable under acidic and basic conditions; (b) Stable under gastrointestinal environments; (c) Optimal hydrophilic/hydrophobic properties; (d) Maximum absorption into enterocytes; (e) Significant percentage of the NO-NSAID dose should be hydrolyzed into NO donor and NSAID in the enterocytes; (f) NO donor should be absorbed to a significant extent. Preferably, a significant percentage of nitric oxide is released from the total NO donor into enterocytes. The concentration of nitric oxide should be high enough to protect the stomach and intestinal tract from irritation, bleeding and ulceration. A significant percentage of the nitric oxide donor should be released in the gastrointestinal tract, preferably, 5 to 50% of the dose equivalent; (g) The NO donor should be adequately hydrolyzed in the plasma and/or endothelial cells to release NO.

In one embodiment, the present invention provides a method of pairing a therapeutic agent with an appropriate nitric oxide donor to create an effective prodrug molecule. The method comprises: (i) obtaining in vitro or in silico pharmacokinetic or pharmacodynamic data, (ii) placing the data into a physiologically-based pharmacokinetic/pharmacodynamic model, and (iii) generating output parameters from the pharmacokinetic/pharmacodynamic model, wherein the output parameters determine the pairing of a therapeutic agent with an appropriate nitric oxide donor to create an effective prodrug molecule.

In one embodiment, the pharmacokinetic model of the present invention comprises (i) a seven compartment model which divides a gastrointestinal tract into seven compartments, wherein said seven compartment model describes gastrointestinal absorption of said prodrug molecule; and (ii) a group of compartment models which divides a body into plasma/blood and tissue compartments (such as heart, kidney, and liver), wherein said group of compartment models describes the time course of the therapeutic agent, the nitric oxide donor, and nitric oxide in gastrointestinal tract, blood, and tissues. Representative in vitro or in silico input data to the model include pKa values, octanol/water partition coefficients, solubility data, permeability values, metabolism data, hydrolysis data, serum protein binding data, nitric oxide release rate, pharmacokinetic and pharmacodynamic data of a therapeutic agent, and stability data in gastric and intestinal environments.

The present invention also provides a prodrug molecule selected by the above method, wherein the prodrug molecule comprises a therapeutic agent and a nitric oxide donor. In general, the therapeutic agent can be a nonsteroidal anti-inflammatory drug or an antibiotic. Representative nonsteroidal anti-inflammatory drugs include, but are not limited to, non-selective cyclooxygenase isozyme inhibitors or cyclooxygenase-2 inhibitors. Examples of non-selective cyclooxygenase isozyme inhibitor include acetylsalicylic acid (CH₃COOC₆H₄COOH), IBUPROFEN (C₁₃H₁₈O₂), NAPROXEN (C₁₄H₁₄O₃,) indomethacin (C₁₉H₁₆ClNO₄), and diclofenac (C₁₄H₁₀Cl₂NNaO). Moreover, the cyclooxygenase-2 inhibitor may comprise a carboxyl group. An example of nitric oxide donor is a diazeniumdiolate such as diazen-1-ium-1,2-diolate. And one of ordinary skill in the art would readily apply an antibiotic as a therapeutic agent in view of the teaching of the present invention.

In another embodiment, there is provided a prodrug molecule comprising a nonsteroidal anti-inflammatory drug and a nitric oxide releasing moiety, wherein the moiety has a half-life that is longer than the total time period for hydrolysis and absorption, and wherein a therapeutic dosage of nitric oxide is released into enterocytes, thereby protecting against damage caused by gastrointestinal irritation, bleeding or ulceration. Moreover, a therapeutic dosage of nitric oxide may be released into blood stream, thereby protecting one or more organ system such as heart, kidney, and cardiovascular system. In general, the therapeutic agent can be a nonsteroidal anti-inflammatory drug or an antibiotic, and an example of a nitric oxide releasing moiety is a diazeniumdiolate such as diazen-1-ium-1,2-diolate.

The present invention also provides a prodrug molecule comprising: (i) a nitric oxide releasing moiety linked to an amino acid through a linkage that is susceptible to enzymatic hydrolysis or cleavage, and (ii) a therapeutic agent directly linked to said amino acid, or linked to said amino acid through a spacer, wherein the linkage between the therapeutic agent and the spacer, or the linkage between the spacer and the amino acid is susceptible to enzymatic hydrolysis or cleavage, wherein the release of the nitric oxide releasing moiety and the therapeutic agent from the prodrug molecule can be controlled independently. In general, the linkage susceptible to enzymatic hydrolysis or cleavage is an ester linkage, thioester linkage, amide linkage, or sulfonamide linkage. The amino acid in this prodrug molecule can be hydroxyproline, glutamic acid, or aspartic acid. Furthermore, the amino acid may also comprise a free or substituted amine or amine salt.

The present invention also provides a compound of the formula I:

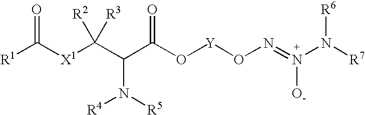

wherein R¹ is an uncarboxylated core of a non-steroidal anti-inflammatory drug, (e.g. naproxen, aspirin, ibuprofen, indomethacin, salicylic acid, mesalamine, flunixin, ketorolac, tolfenamic acid, niflumic acid, mefenamic acid, meclofenamic acid, flufenamic acid, enfenamic acid, etodolac, pirazolac, tolmetin, bromofenac, fenbufen, mofezolac, diclofenac, pemedolac, sulindac, suprofen, ketoprofen, tiaprofenic acid, fenoprofen, indoprofen, carprofen, loxoprofen, ibuprofen, pranoprofen, bermoprofen, zaltoprofen, flurbiprofen, tenoxicam, piroxicam, meloxicam, lornoxicam, tenidap, paracetamol, salactamide); or a structure of the formula II:

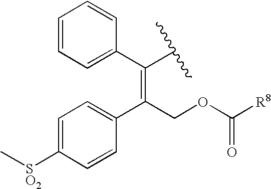

wherein R⁸ is hydrogen, an unsubstituted or substituted C₁₋₁₂ straight chain alkyl, an unsubstituted or substituted C₃₋₁₂ branched chain alkyl.

X in the formula I can have a structure of the formula III:

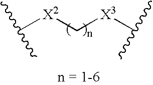

wherein X² is oxygen, sulfur, or NH and X³ is oxygen, sulfur, or NH.

Alternatively, X¹ in the formula I can have a structure of the formula IV:

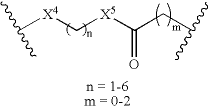

wherein X⁴ is oxygen, sulfur, or NH and X⁵ is oxygen, sulfur, or NH.

In another embodiment, X¹ in the formula I can have a structure of the formula V:

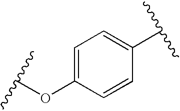

In yet another embodiment, X¹ in the formula I can have a structure of the formula VI:

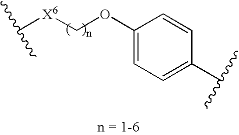

where X⁶ is oxygen, sulfur, or NH.

R² in the formula I can be hydrogen, an unsubstituted or substituted C₁₋₁₂ straight chain alkyl, an unsubstituted or substituted C₃₋₁₂ branched chain alkyl.

R³ in the formula I can be hydrogen, an unsubstituted or substituted C₁₋₁₂ straight chain alkyl, an unsubstituted or substituted C₃₋₁₂ branched chain alkyl.

R⁴ in the formula I can be hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl; or a structure of the formula VII:

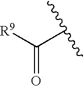

wherein R⁹ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl; an amide derivative linked via a carboxy group of an amino acid e.g. β-alanine, alanine, 2-aminobutyric acid, 6-aminocaproic acid, α-aminoisobutyric acid, α-aminosuberic acid, arginine, asparagines, aspartic acid, citrulline, β-cyclohexylalanine, cysteine, 3,4-dehydroproline, glutamic acid, glutamine, glycine, histadine, homocitrulline, homoserine, hydroxyproline, β-hydroxyvaline, isoleucine, leucine, lysine, methionine, norleucine, norvaline, ornithine, penicillamine, phenylalanine, phenylglycine, proline, pyroglutamine, sarcosine, serine, statine, threonine, tryptophan, tyrosine, valine, or an amide derivative of a polypeptide.

In another embodiment, R⁴ in the formula I is a structure of the formula VIII:

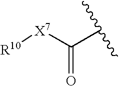

wherein X⁷ is oxygen, sulfur, or NH, and R¹⁰ is an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl.

In yet another embodiment, R⁴ in the formula I is a structure of the formula IX:

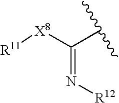

wherein X⁸ is oxygen, sulfur, or NH; and R¹¹ is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl; and R¹² is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl.

R⁵ in the formula I can be hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl; a structure of formula VII, a structure of formula VIII, or a structure of formula IX.

R⁶ in the formula I can be hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII.

R⁷ in the formula I can be hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII, or NR⁶R⁷ is a cyclic heterocycle of the formula X:

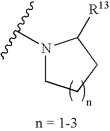

wherein R¹³ is hydrogen, or a structure of formula XI:

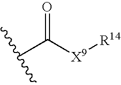

wherein X⁹ is oxygen, sulfur, or NH, and R¹⁴ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, an amino acid wherein X⁹ is the amino group of the amino acid (e.g. b-alanine, alanine, 2-aminobutyric acid, 6-aminocaproic acid, a-aminoisobutyric acid, a-aminosuberic acid, arginine, asparagines, aspartic acid, citrulline, b-cyclohexylalanine, cysteine, 3,4-dehydroproline, glutamic acid, glutamine, glycine, histadine, homocitrulline, homoserine, hydroxyproline, b-hydroxyvaline, isoleucine, leucine, lysine, methionine, norleucine, norvaline, ornithine, penicillamine, phenylalanine, phenylglycine, proline, pyroglutamine, sarcosine, serine, statine, threonine, tryptophan, tyrosine, valine, or a polypeptide linked via an amino functional group).

Alternatively, NR⁶R⁷ is a cyclic heterocycle of the formula XII:

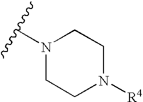

wherein Y is a structure of the formula XIII:

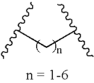

wherein Y is a structure of the formula XIV:

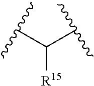

wherein R¹⁵ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl.

The present invention also provides a compound of the formula XV:

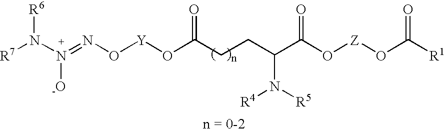

wherein Z is a structure of the formula XIII, or a structure of the formula XIV.

The present invention also provides a compound of the formula XVI:

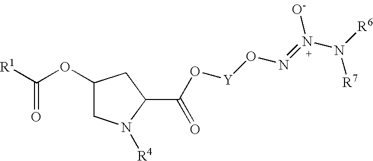

The present invention also provides a compound of the formula XVII

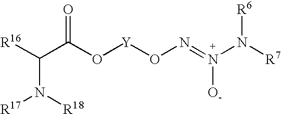

where the sub structure of the formula XVII

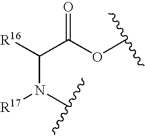

represents the core structure of the amino acids alanine, 2-aminobutyric acid, acid, α-aminosuberic acid, arginine, asparagines, aspartic acid, citrulline, β-cyclohexylalanine, cysteine, 3,4-dehydroproline, glutamic acid, glutamine, glycine, histadine, homocitrulline, homoserine, hydroxyproline, β-hydroxyvaline, isoleucine, leucine, lysine, methionine, norleucine, novaline, ornithine, penicillamine, phenylalanine, phenylglycine, proline, pyroglutamine, sarcosine, serine, threonine, tryptophan, tyrosine, or valine; wherein R¹⁸ is a structure of the formula XVIII

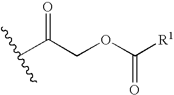

Alternatively, R¹⁸ is a structure of the formula XIX:

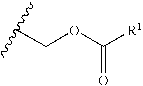

or R¹⁸ is a structure of the formula XX:

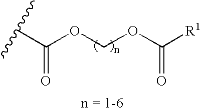

or R¹⁸ is a structure of the formula XXI:

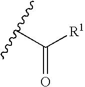

The present invention also provides for a structure of the formula XXII:

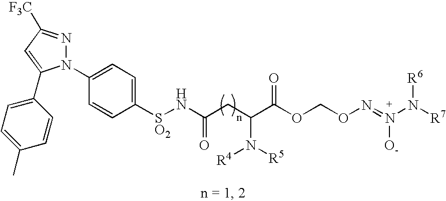

The present invention also provides a structure of the formula XXIII:

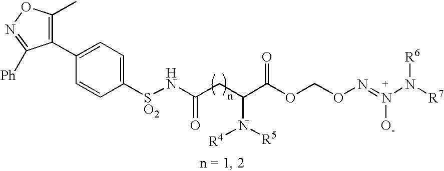

Compounds of the present invention which contain one or more asymmetric atoms can exist and be used as optically pure enantiomers, mixtures of enantiomers, mixtures of enantiomers of pure diastereomers, mixtures of both enantiomers and diastereomers, completely racemic mixtures. Compounds of the present invention which contain one or more carbon-carbon double bonds may exist as pure E or Z isomers or mixtures of these isomers. Compounds of the invention which contain one or more carbon-nitrogen double bonds may exist as pure E or Z isomers or mixtures of these isomers. Compounds of the invention which contain one or more atropisomers may contain pure isomers or mixtures of these isomers. The present invention anticipates and includes all such isomers and mixtures thereof.

Compounds of the present invention which contain at least one functional group salifiable with acids (e.g. primary, secondary or tertiary amines) can be transformed into the corresponding salts. Organic acids which could be used in this capacity include oxalic, tartaric, maleic, succinic, citric, trifluoroacetic acids. Examples of inorganic acids which could be used in this capacity are nitric, hydrochloric, sulfuric and phosphoric acids.

The invention being generally described, will be more readily understood by reference to the following examples which are included merely for purposes of illustration of certain aspects and embodiments of the present invention, and are not intended to limit the invention.

### EXAMPLE 1

### Selecting Appropriate NO-NSAID Candidate

The main objectives of this example are to (1) provide in vivo data (i.e. NSAID and NO kinetic data) to train the in silico manifestation of the pharmacokinetic/pharmacodynamic model; (2) validate model predictions; and (3) select appropriate NO-NSAID candidate(s) for future development. An NO-NSAID candidate will be declared as a lead when it shows a potential of maintaining its original NSAID anti-inflammatory activity, backed up by NO production and PK data, without its untoward gastrointestinal, cardiovascular and kidney events.

Anti-inflammatory activities of non-selective and selective NSAIDs were indirectly measured using biomarkers indicating their ability to inhibit COX-1 and -2 activities. NO activities were measured that were relevant to their potential ability to counteract NSAID side effects such as cardiovascular and kidney events.

Myocardial infarction and ischemic events in high risk patients led one of the most popular COX-2 inhibitors to its demise (Bresalier et al., 2005). Non-selective NSAIDS may also cause similar problems due to their ability to inhibit COX-2. It has been postulated that a shift in the ratio of PGI₂ to thromboxane A₂ during NSAID treatment would provide early indications of atherogenicity (Krötz et al., 2005). Adenosine diphosphate (ADP) generation is an indicator of myocardial infarction which has no connection with the arachidonic acid cascade (Borna et al., 2005). ADP level has been shown to be lowered by NO. Long term NSAID use has been linked to kidney damage and hypertension (Zafirovska et al., 1993). After a single dose of NSAID, proximal tubular damage has been demonstrated (Porter et al., 1999). This was associated with an increase in the urine the ratio of alanine-amino-peptidase (AAP) and creatinine.

Table 1 is a summary of the protocol for a study which was conducted in male Wistar rats weighing 275-300 grams. The animals were allowed to acclimatize for at least five days prior to the commencement of the study. The study protocol was approved by the local animal ethics committee.

TABLE 1
A Protocol for Evaluating NAPROXEN Derivative Diazeniumdiolate
NO-NSAID Candidates

| | | No. of | | | |
|---|---|---|---|---|---|
| | | Animals | Dosage | Dosage | Dosage |
| | | per | Level | Concentration | Volume |
| Treatment Group | Treatment | Group | (mg/kg) | (mg/mL) | (mL/kg) |
| VEH | vehicle | 5 | NA | NA | 5 |
| NAP | naproxen | 5 | 250 | 50 | 5 |
| PYRO/NO- | pyrrolidino- | 5 | Molar | Specified in | 5 |
| NAP | diazeniumdiolate- | | equivalent | raw data | |
| | naproxen | | to the NAP | | |
| | | | group | | |
| DMA/NO- | dimethylamino- | 5 | Molar | Specified in | 5 |
| NAP | diazeniumdiolate- | | equivalent | raw data | |
| | naproxen | | to the NAP | | |
| | | | group | | |
| AZD3582 | AstraZeneca/Nicox- | 5 | Molar | Specified in | 5 |
| | naproxen nitrate | | equivalent | raw data | |
| | ester 2 | | to the NAP | | |
| | | | group | | |
| ROF | Rofecoxib 10 | 5 | 250 | 50 | 5 |

The animals were fasted overnight prior to test substance administration. The test substance was administered orally by gavage on the morning of the study day. All test animals had blood collected during the test period in both EDTA (ethylenediaminetetraacetic acid) tubes and SST (Serum Separator Tubes) at 1, 3 and 6 hours after dosing. Blood was collected into EDTA tubes only at 1 and 3 hours by tail tip amputation. Volume of blood collected was between 0.5 and 1 mL at each of these collections. The final collection was by puncture of the abdominal vena cava under isoflurane general anesthesia. Final blood collection was targeted as follows: 1^{st} EDTA #1—1.5 mL; 2^{nd} SST—1.5 mL; 3^{rd} EDTA #2—as much as possible.

EDTA samples were centrifuged, and the plasma portion was collected and frozen at −80° C. SST samples were incubated at 37° C. for approximately 45 minutes, centrifuged, and the serum was collected and frozen at −80° C. until analysis.

All test animals, housed in metabolic cages, had urine collected over wet ice for 6 hours after dosing. Aliquots of the urine from each animal were collected to determine creatinine concentration. The remainder of the urine was centrifuged at 1000 g for 10 minutes. The supernatant was collected and mixed with analytical grade ethylene glycol at a rate of 0.4 mL ethylene glycol per 1 mL urine supernatant. The ethylene glycol/urine mixture was stored at −80° C. until analysis.

All rats were euthanized by removal of the heart 6 hours after initial dosing, following final blood collections. Immediately after terminal blood collections were complete, the thorax was opened and the heart removed. The heart was cut in half longitudinally. Half of the heart was placed in formalin for histological examination. The other half was quickly rinsed in saline and then freeze-clamped (crushed between the jaws of a pair of modified tongs, cooled by liquid nitrogen). An entire kidney was freeze-clamped. The other kidney was left in situ for examination by the pathologist. Freeze-clamped tissues were wrapped in labeled aluminum foil and stored in liquid nitrogen until they could be transferred to a −80° C. freezer. Freeze-clamped tissues were shipped on dry ice to NOVOKIN for analysis.

Each animal from all groups underwent a full necropsy under the supervision of a board certified veterinary pathologist. The stomach of each rat was cut along the greater curvature, contents removed into a polypropylene (Falcon) tube, the mucosa rinsed with saline and any obvious ulcers or erosions were measured along the longest axis. This measurement was recorded for each ulcer or erosion observed in each stomach. The falcon tubes and their contents were frozen at −80° C. and shipped to Novokin on dry ice for analysis.

The stomach, duodenum, jejunum, ileum, cecum, colon, liver, kidneys and heart were examined and collected into 10% neutral buffered formalin. Two stomachs from each group regarded as being representative of that group had their mucosal surfaces photographed. Other tissues were also photographed.

The above tissues were all processed for histopathological examination using hematoxylin and eosin staining. Additional unstained slides were provided to the sponsor for TUNEL staining.

The aggregate length of all gastric ulcers found in a given animal was calculated. The mean aggregate ulcer length across animals in a group was calculated. This mean value was reported as the ulcer index for that group.

The ulcer index of each modified drug was compared with the ulcer index of its associated parent drug and the controls using analysis of variance and Duncan's multiple range test for pairwise comparisons. Statistical analysis was done using SAS® (SAS Institute Inc., Cary, N.C.).

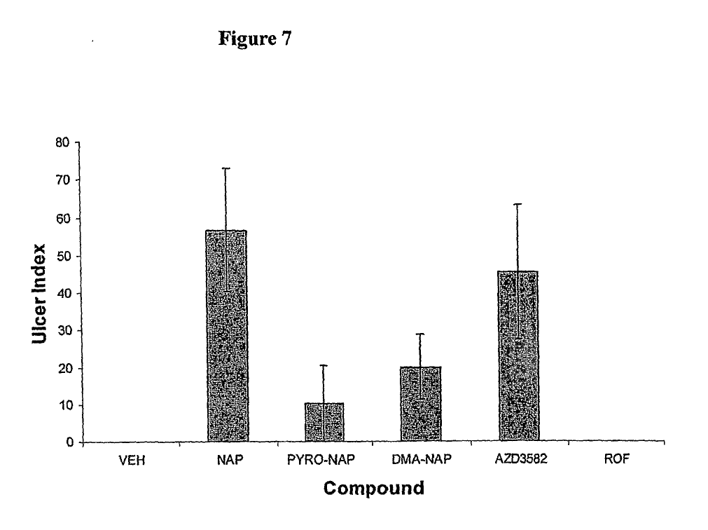

The results of this study showed that naproxen and AZD3582 were severely ulcerogenic (Table 2, FIG. 7). DMA/NO-NAP was somewhat less ulcerogenic, while PYRO/NO-NAP and ROF were not significantly different from vehicle.

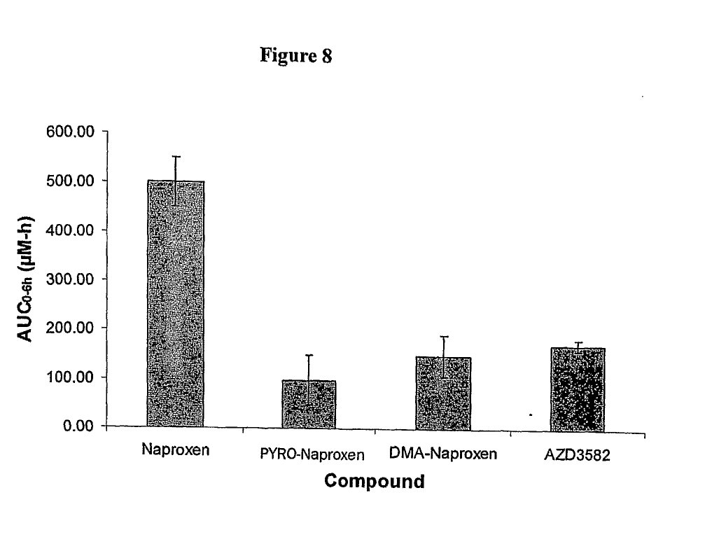

AUC₀₋₆ₕ (μM-h) values for NAPROXEN after dosing with the prodrug candidates or AZD3582 (Table 3, FIG. 8) were much lower than for NAPROXEN parent drug, indicating that all three compounds had poorer absorption than the parent drug.

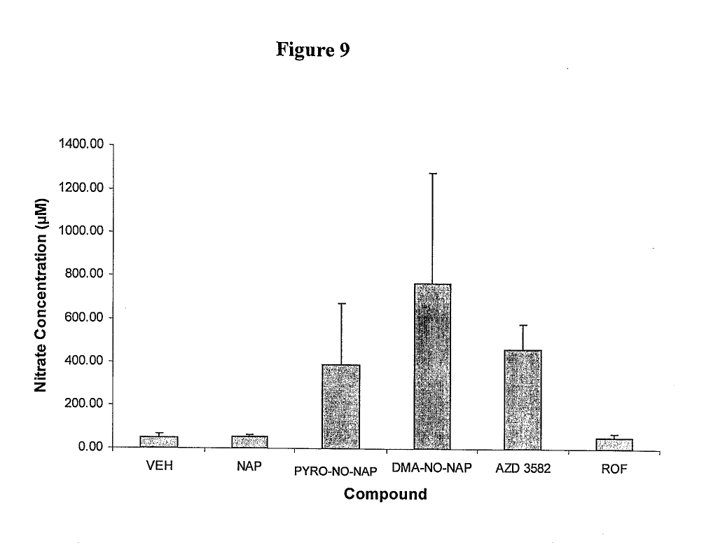

Release of NO as indicated by the serum nitrate concentrations at 6 hours (Table 4, FIG. 9) was similar among the NO donors but erratic, resulting mostly in trends toward higher concentrations even though the means were several-fold higher than ROF or VEH.

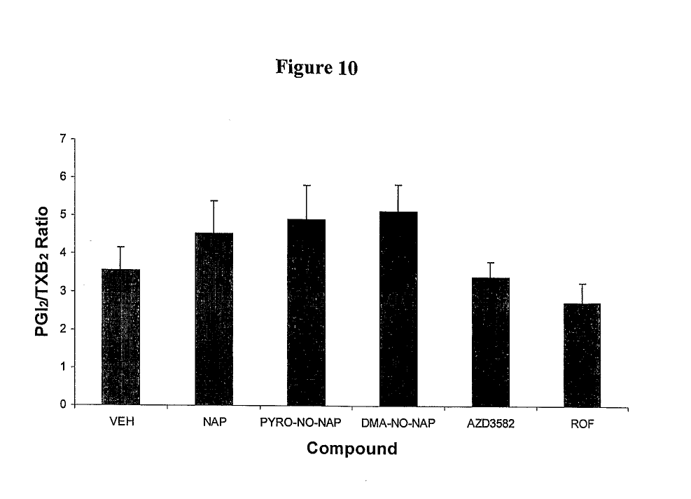

NAP lowered both PGI2 and TXA2 levels (Table 5, FIG. 10), leading to an insignificant change in the PGI2/TXA2 ratio when compared to control (p>0.05). Both diazeniumdiolate compounds have higher ratios than NAP with DMA-NO-NAP ratios achieving statistical significance (p<0.05), suggesting decreased cardiovascular risk. In contrast, both ROF, in keeping with literature reports, but also AZD3582 exhibited lower ratios than vehicle, suggesting possible increased risk although this was only a trend in this experiment. The cardiac ADP levels and all of the other potential nucleotide levels and ratios indicating energetic stress on the heart remained the same across all groups, indicating that this biomarker is insensitive to any particular toxicity exerted by this group of compounds (data not shown).

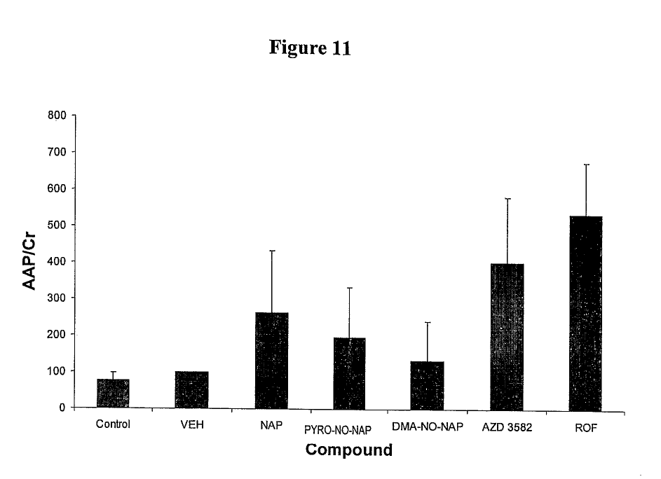

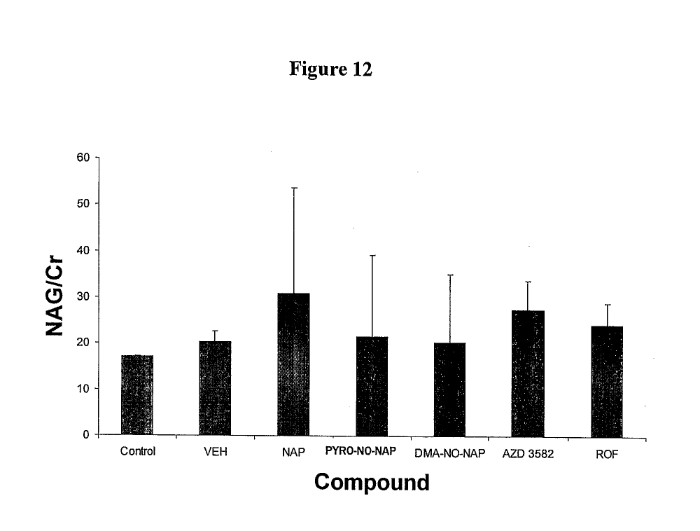

NAPROXEN and its diazeniumdiolate prodrug compounds caused no significant increase in the urine AAP/creatinine ratio (Table 6, FIG. 11) while ROF and AZD3582 did cause an increase in this biomarker, indicating proximal tubular kidney damage by these two compounds. None of the compounds caused a significant change in NAG/cr (Table 7, FIG. 12), indicating a lack of toxicity toward distal tubules.

In summary, the new NO-NSAID candidates appeared to be effective and have the following advantages over ROFECOXIB and AZD3582: (a) plasma nitrite and nitrate levels increased; (b) ulcer indices were lower than that of NAPROXEN or AZD3582 but not ROFECOXIB treated animals. The results were similar to that of the control for the new compounds; (c) cardiac PGI2/TXB2 was higher than vehicle for NAPROXEN and the new compounds, and lower for ROFECOXIB and AZD3582 suggesting a cardiotoxicity benefit with diazeniumdiolate NO-donors; (d) urine AAP/creatinine ratio was similar to that of the control and lower than that of the AZD3582 and ROFECOXIB treated animals, suggesting a renal toxicity benefit to the diazeniumdiolates.

These results were compared to those of the corresponding in vitro microsomal data. Because the NO-NSAID candidate is estimated to release NO completely in the gut and the in vivo data show that the new compounds have cardiac and kidney effects, it suggests that NO is being transported at least somewhat by carriers such as nitrosothiols and NO-hemoglobin. This comparison provided information that is of tremendous value in the future design of NO-NSAID and input value of the pharmacokinetic/pharmacodynamic model.

This example suggests that in spite of potential NO-related benefits shown in each biomarker category, the actual reproducibility and degree of improvement may not be sufficient to ensure commercial success of these compounds. The reason is that the dosage used in this study is in the toxic range and the amount of NO release will be at least several times lower at clinical doses. An in vitro study (Knaus et al., 2005) showed that the diazeniumdiolate candidates used in this study have 13 times the capacity of producing NO when compared to the candidates such as AZD 3582 which contains organic nitrates as NO donors. Interestingly, the nitrate levels observed in this study is nowhere close; suggesting a lot of NO has been “wasted” in vivo (FIG. 9). Therefore, it will be necessary to further improve the solubility, permeability and NO release characteristics over the studied compounds before taking a new compound through the development process. In particular, a more stable or stabilized NO donor to link to NAPROXEN would improve systemic delivery and hence cardiac and renal distribution/NO exposure of these organs, increasing the benefit derived from the NO. This example demonstrates the necessity for improvements to the Knaus et al. molecules, and these can effectively and efficiently be directed through the use of pharmacokinetic/pharmacodynamic modeling conducted in silico.

TABLE 2
Results of Duncan's Multiple Range Test For Pairwise Comparisons
of Ulcer Indices

| Category | Mean | Groups | | | | |
|---|---|---|---|---|---|---|
| VEH | 0.000 | A | | | | |
| ROF | 0.000 | A | B | | | |
| PYRO-NO-NAP | 1.450 | | B | C | | |
| DMA-NO-NAP | 1.695 | | | C | D | |
| AZD 3582 | 3.444 | | | | D | E |
| NAP | 4.282 | | | | | E |

Pairs with the same letter are not significantly different (p > 0.05).

TABLE 3
Results of Duncan's Multiple Range Test For Pairwise Comparisons
of AUC₀₋₆ₕ (μM-h) Values

| Category | Mean | Groups | | |
|---|---|---|---|---|
| PYRO-NO-Nap | 99.262 | A | | |
| DMA-NO-Nap | 150.570 | A | B | |
| AZD 3582 | 173.168 | | B | |
| Naproxen | 501.606 | | | C |

Pairs with the same letter are not significantly different (p > 0.05).

TABLE 4
Results of Duncan's Multiple Range Test For Pairwise Comparisons
of Serum Nitrate Concentrations

| Category | Mean | Groups | | |
|---|---|---|---|---|
| VEH | 51.424 | A | | |
| NAP | 55.167 | A | B | |
| ROF | 55.818 | A | B | C |
| PYRO-NO-NAP | 389.000 | A | B | C |
| AZD 3582 | 460.667 | | B | C |
| DMA-NO-NAP | 768.000 | | | C |

Pairs with the same letter are not significantly different (p > 0.05).

TABLE 5
Results of Duncan's MULTIPLE RANGE TEST FOR PAIRWISE
COMPARISONS of Cardiac Tissue PGI2/TXB2

| Category | Mean | Groups | | |
|---|---|---|---|---|
| ROF | 2.765 | A | | |
| AZD 3582 | 3.432 | A | | |
| VEH | 3.562 | A | B | |
| NAP | 4.544 | | B | C |
| PYRO-NO-NAP | 4.937 | | | C |
| DMA-NO-NAP | 5.157 | | | C |

Pairs with the same letter are not significantly different (p > 0.05).

TABLE 6
Results of Duncan's Multiple Range Test For Pairwise Comparisons
of AAP/Cr.

| Category | Mean | Groups | | |
|---|---|---|---|---|
| VEH | 100.000 | A | | |
| DMA-NO-NAP | 132.412 | A | B | |
| PYRO-NO-NAP | 195.569 | A | B | |
| NAP | 263.788 | A | B | C |
| AZD 3582 | 403.400 | | B | C |
| ROF | 538.367 | | | C |

Pairs with the same letter are not significantly different (p > 0.05).

TABLE 7
Results of Duncan's Multiple Range Test For Pairwise Comparisons
of Urine NAG/Cr values

| Category | Mean | Groups |
|---|---|---|
| VEH | 16.193 | A |
| DMA-NO-NAP | 20.403 | A |
| PYRO-NO-NAP | 21.515 | A |
| ROF | 24.176 | A |
| AZD 3582 | 27.439 | A |
| NAP | 30.865 | A |

Pairs with the same letter are not significantly different (p > 0.05).

### EXAMPLE 2

### Physiologically-Based Pharmacokinetic/Pharmacodynamic Model

The objective of this example is to demonstrate an embodiment of an in silico physiologically-based pharmacokinetic computer model which incorporates all of the principal processes and parameters and which is able to generate output as described.

The model consists of a number of compartments, each representing a specific anatomic region. For each compound of interest in the model, each compartment has a specific volume (volume of distribution) and has a uniform interior concentration (“well-stirred” condition) of the compound (FIG. 13). Compounds are transported between compartments with rates proportional to the amount of material in the originating compartment (first-order kinetics). These transports reflect diffusion and bulk flows between physiologically adjoining compartments. Many of these transports represent blood plasma circulation, and the sum of these flows into any compartment is equal to the sum of all blood plasma flows out of the compartment. Within a compartment, compounds undergo metablokic reactions, producing metabolites in stoichiometric proportions, at compound and compartment specific rates. The new materials are produced in amounts proportional to those of the original compounds (first-order kinetics)

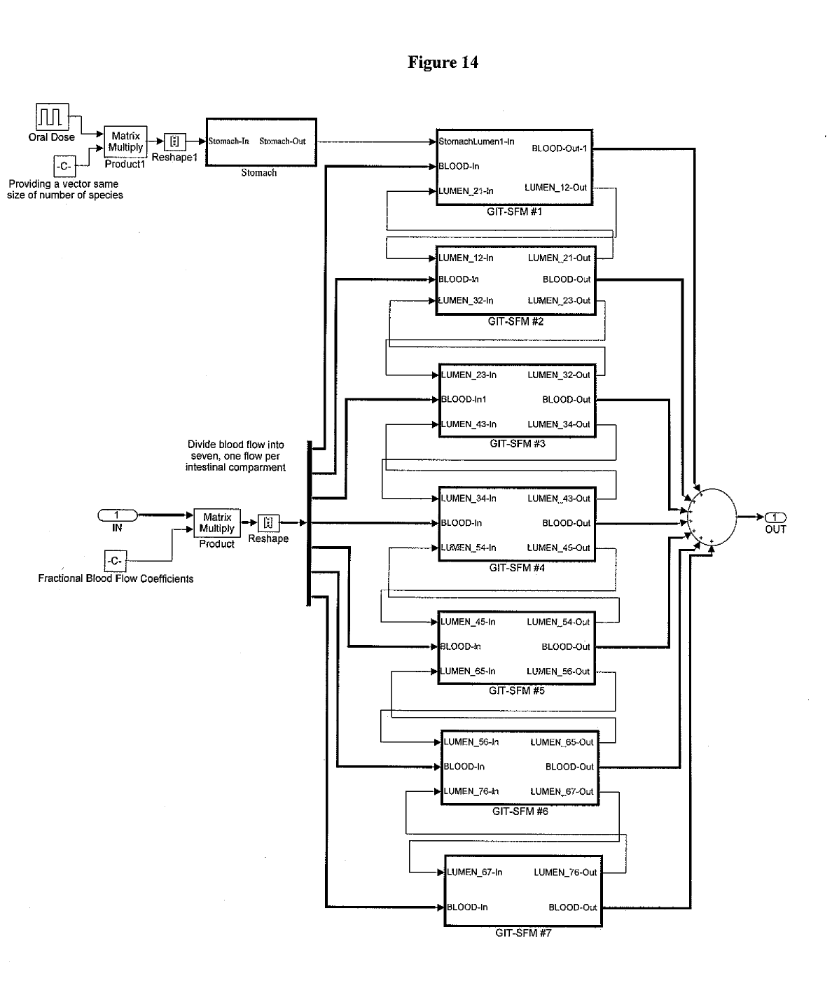

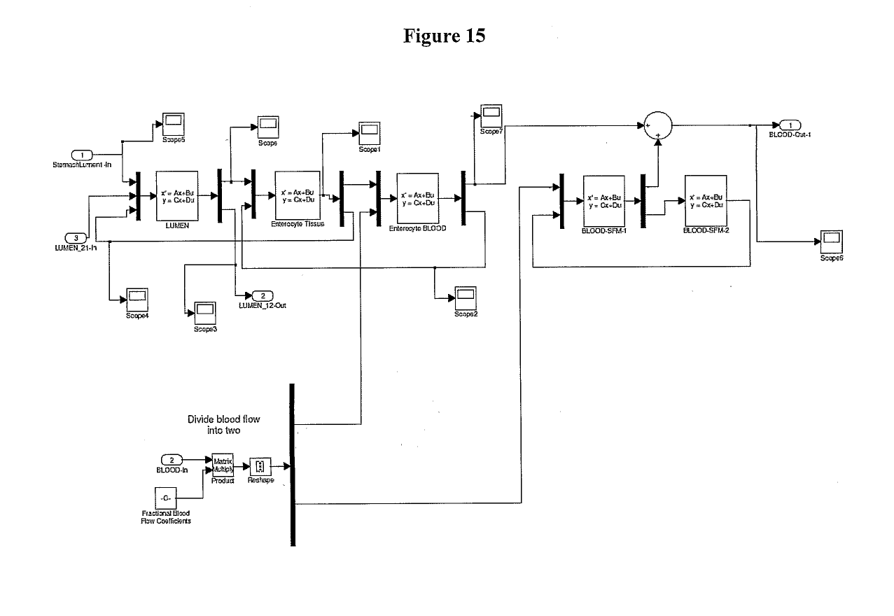

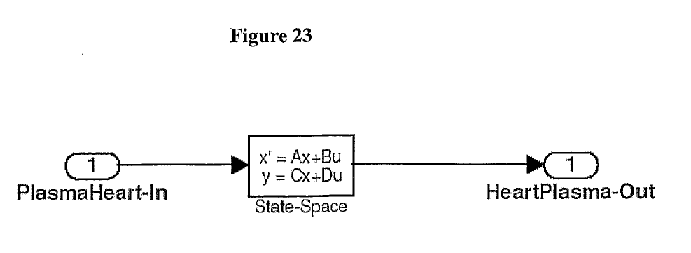

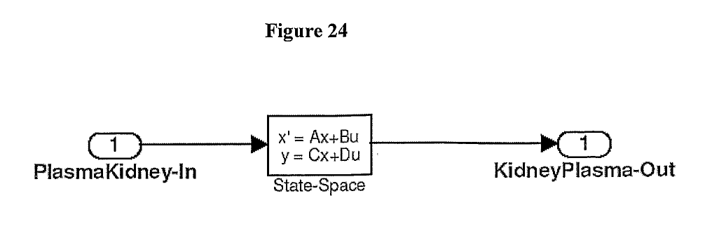

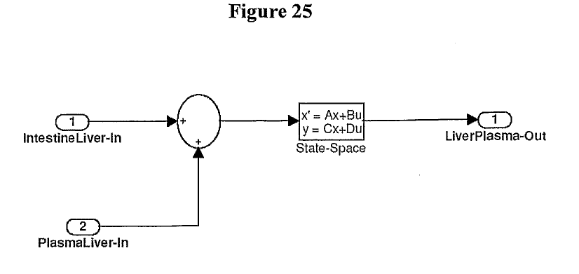

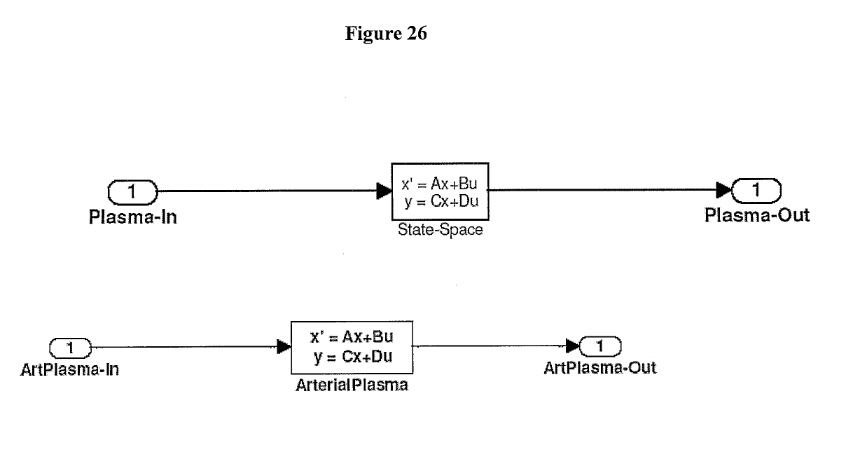

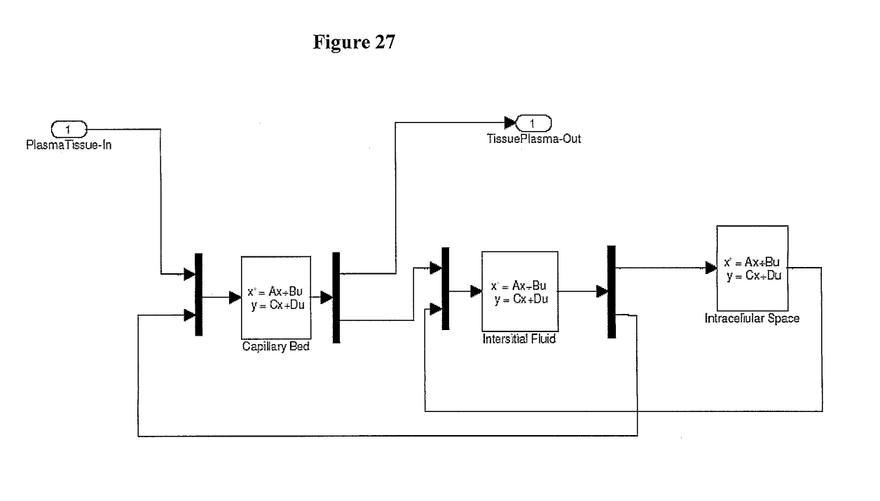

The simulation consists of an arterial blood plasma compartment (FIG. 26) with flows to an intestinal region (FIG. 14). Flows lead from the intestinal region to a compartment representing the liver (FIG. 25). The arterial blood plasma compartment also has flows to four compartments representing heart (FIG. 23), liver (FIG. 25), kidney (FIG. 24), and other tissues (FIG. 27), respectively. Flows lead from these four compartments into a venous blood plasma compartment. This has a flow into a lung compartment, which in turn flows into the arterial blood plasma compartment. The intestinal region is divided into seven segments, each comprised of five compartments (FIGS. 15 to 21). One of the five compartments of each intestinal segment represents the intestinal lumen and these luminal compartments are connected in sequence to reproduce drug transit including peristaltic behavior of the intestine. Within each of the seven intestinal segments, the lumen compartment has bi-directional flows with an enterocyte (absorptive cells lining the lumen) compartment, which also has bi-directional flows with a blood plasma compartment. A second blood plasma compartment has bi-directional flows with the fifth compartment representing the other intestinal tissues supplied by the cranial mesenteric artery. Each blood plasma compartments receives an in-flow from the arterial compartment has an equal output to the hepatic compartment. No other intestinal compartments are connected to the rest of the model. A final compartment with a flow into the first lumen compartment is used to model an oral dose of drug (FIG. 15). Physiological data from human and animals for body weight, cardiac output, blood flow to various organs, volumes and weight of each organ, extracellular and intracellular fluid, lipids, were collected from the literature and are summarized in following tables. In silico estimation of log P, plasma protein binding, and per organ volumes of distribution, is accomplished using the methods published by Ghose et al. (1998), Lobell and Sivarajah (2003), and Poulin and Thiel, (2002), respectively.

Whenever in vitro estimate is attainable, the in vitro results will be used, for example, plasma protein binding. Methods reported by Bowalgaha & Miners (2001), Martignoni et al. (2006), Tong et al. (2001) and Thulesen et al. (1999) were used for in vitro and in vivo scale-up for clearance in the intestine, absorption rate constant and hepatic clearance. The simulation begins with no material in all compartments except for the initial bolus in one compartment (typically the stomach compartment). The simulation then estimates the changing distribution of the material with time.

The current version of the simulation is implemented using the MatLab and its Simulink Toolbox (both The Mathworks, Natick, Mass.), and is a mixture of the Simulink graphical model interface, MatLab command language, and a code-generation routines written in Perl. The structure of the model is depicted in FIGS. 13-28.

### EXAMPLE 3

### Effects of NSAIDs and NO Donors on Platelet Aggregation

The objectives of this example are to: (1) study the effects of NSAIDs and NO donors on platelet aggregation, vasodilation, and thrombus formation; (2) study the potential interaction between NSAIDs and NO donors in platelet aggregation, vasodilation and thrombus formation; and (3) the effects of NSAIDs and NO on COX functions.

The methods published by Al et al. (2006), Hanson et al. (2005), Turkan et al. (2004) and Tubara et al. (2001) will be used to achieve these objectives.

In vitro results obtained from these studies will be used to simulate the time course of platelet aggregation, vasodilation and thrombi formation after administration of NO-NSAID candidates.

### EXAMPLE 4

### Training of the Pharmacokinetic/Pharmacodynamic Model

The objectives of this example are: (1) to train the physiologically-based pharmacokinetic/pharmacodynamic (PBPK/PD) model and (2) to use the PBPK/PD model to predict in vivo pharmacokinetic and pharmacodynamic behavior of potential candidates in human and rat. The physiological, in vitro and in silico inputs into the model are listed in Table 8.

NAPROXEN, AZD 3582 and PYOR/NO-NAPROXEN are used to train the model. The in vitro parameters are generated in house unless they are specified otherwise. The model parameters are listed in Table 9.

TABLE 8
Input for the Physiologically-Based
Pharmacokinetic/Pharmacodynamic Model

| Parameter name | Rat | Human | Source |
|---|---|---|---|
| Body mass, kg | 0.25 | 70 | |
| Stomach Volume, mL | 1.1 | 155 | Bernareggi and Rowland, 1991 |
| Relative small intestine | 1.40 | 0.91 | Tables 5 & 7 - Brown et al., 1997 |
| weight, % body weight | | | |
| Total lumen volume mL | 2.0 | | Pang, 2003 |
| Total plasma volume, enterocyte, | 0.9 | | Pang, 2003 |
| mL | | | |
| Total plasma volume, serosa, mL | 0.81 | | Pang, 2003 |
| Intestinal enterocyte/serosa ratio | 1.11 | * | Pang, 2003 |
| Intestinal radius, cm | 0.2 | 2.0 | RIVM report, 1999 |
| Stomach to lumen clearance, | 100 | 100 | arbitrary, fast value |
| mL/min | | | |
| Clearance to next lumen segment, | 0.1 | 0.1 | based 0.03 mL/min in Pang, 2002 |
| mL/min | | | |
| Cardiac output, blood, mL/min | 110 | 5200 | Table 22 - Brown et al., 1997 |
| Plasma fraction of blood | 0.58 | 0.58 | Table II - Davis, 1993 |
| Fraction of aortic blood to GIT | 0.153 | 0.181 | Table 23 - Brown et al., 1997 |
| Fraction of aortic blood to hepatic | 0.02 | 0.046 | ″ |
| artery | | | |
| Fraction of aortic blood to kidneys | 0.141 | 0.175 | ″ |
| Fraction of aortic blood to heart | 0.051 | 0.04 | ″ |
| Fraction of intestinal blood to | 0.3 | * | Pang, 2003 |
| enterocytes | | | |

* Values reported for the rat were used in man.

TABLE 9
Input Parameters for the Simulation of AZD 3582, PYRO/NO-NAP (N-119)
and NAPROXEN.

| Test Substances | Rat | Human | Source |
|---|---|---|---|
| AZD3582 intestinal microsomal | 0.09 | 554 | In-house data |
| activity, mL/min/organ | | | |
| AZD3582 liver microsomal | 1029 | 118566 | In-house data |
| activity, mL/min/organ | | | |
| AZD3582 intestinal permeability, | 30 × 7.0e⁻⁶ | 15 × 6.0e⁻⁶ | Fagerholm et al., 2005 |
| cm/s | | | (human paper) |
| AZD3582 to Naproxen, intestinal | 10 | 36 | In-house data |
| molar percent conversion, % | | | |
| AZD3582 to Naproxen, liver | 40 | 72 | In-house data |
| molar percent conversion, % | | | |
| AZD3582 gastric decay rate | 0 | 0 | In-house data |
| constant, /mint | | | |
| AZD3582 intestinal lumen decay | 0 | 0 | In-house data |
| rate constant, /min | | | |
| AZD3582 plasma decay rate | 1.0 | 1.0 | In-house data |
| constant, /min | | | |
| N-119 intestinal microsomal | 1 | 188 | In-house data |
| activity, mL/min/organ | | | |
| N-119 liver microsomal activity, | 1920 | 567000 | In-house data |
| mL/min/organ | | | |
| N-119 to Naproxen, intestinal | 10 | 87 | In-house data |
| molar percent conversion, % | | | |
| bN-119 to Naproxen, liver molar | 40 | 72 | In-house data |
| percent conversion, % | | | |
| N-119 gastric decay rate constant, | 0 | 0 | In-house data |
| /mint | | | |
| N-119 intestinal lumen decay rate | 0.025 | 0.025 | In-house data |
| constant, /min | | | |
| N-119 plasma decay rate constant, | 1.4 | — | In-house data |
| /min | | | |
| Naproxen intestinal | 5e⁻⁵ | 8e⁻⁴ | Fagerholm et al., 2005 |
| permeability*, cm/s | | | |
| Naproxen liver microsomal | 0.0075 | 0.0092 | Estimated from Table II- |
| activity, mL/min/organ | | | Runkle, 1972 |

** NAPROXEN has zero permeability from intestinal enterocyte to lumen.

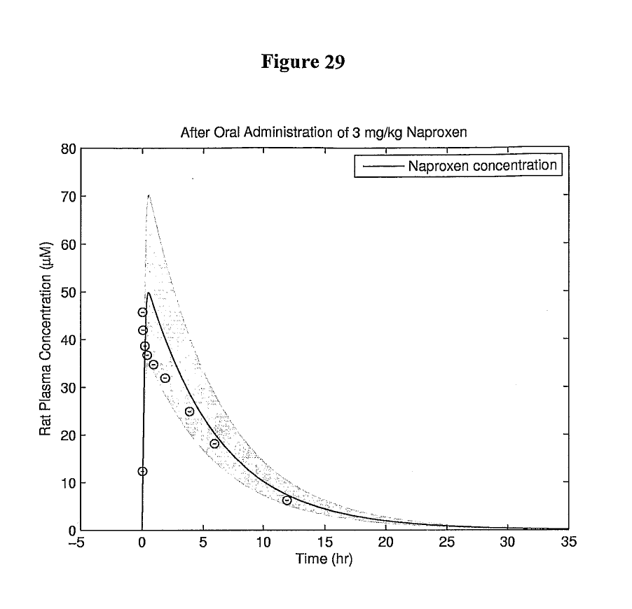

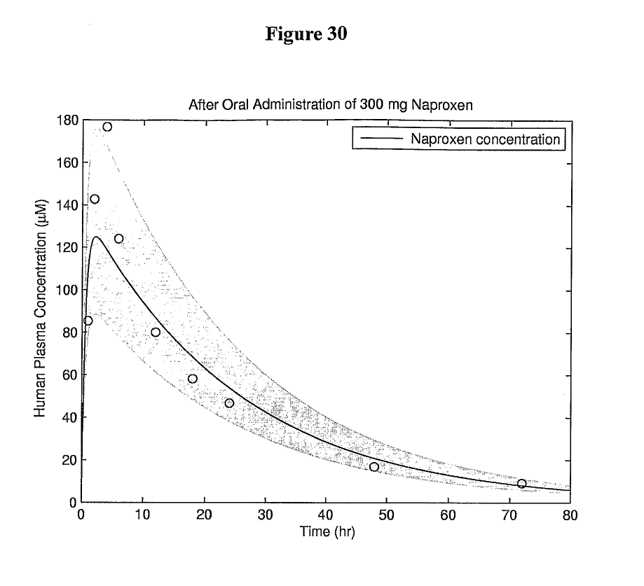

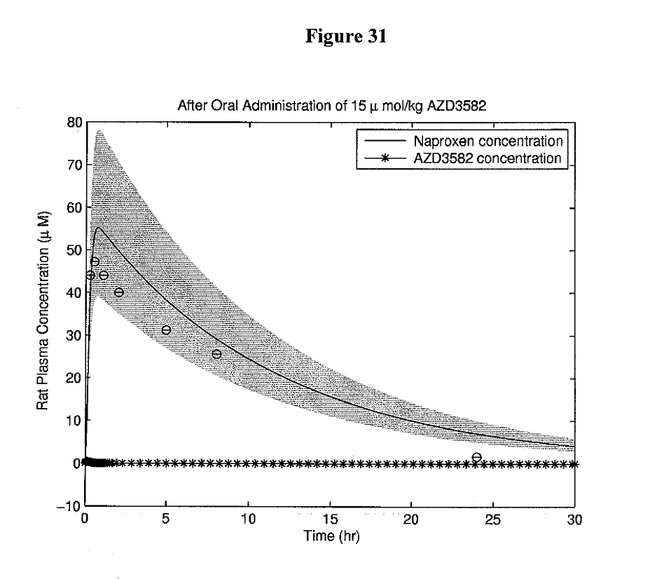

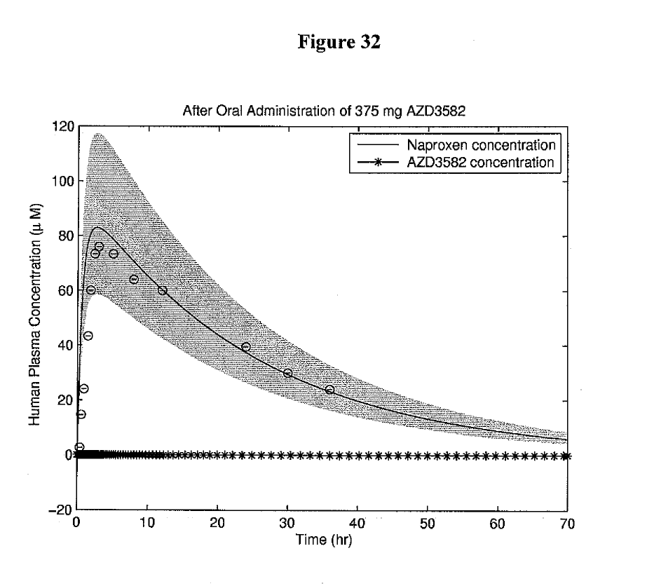

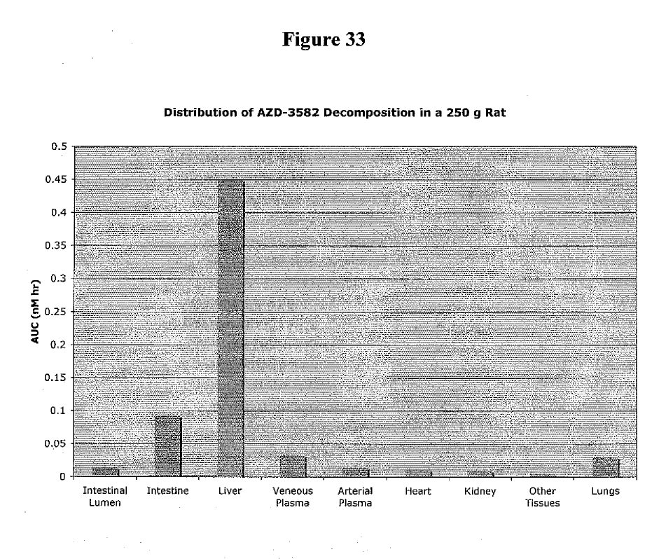

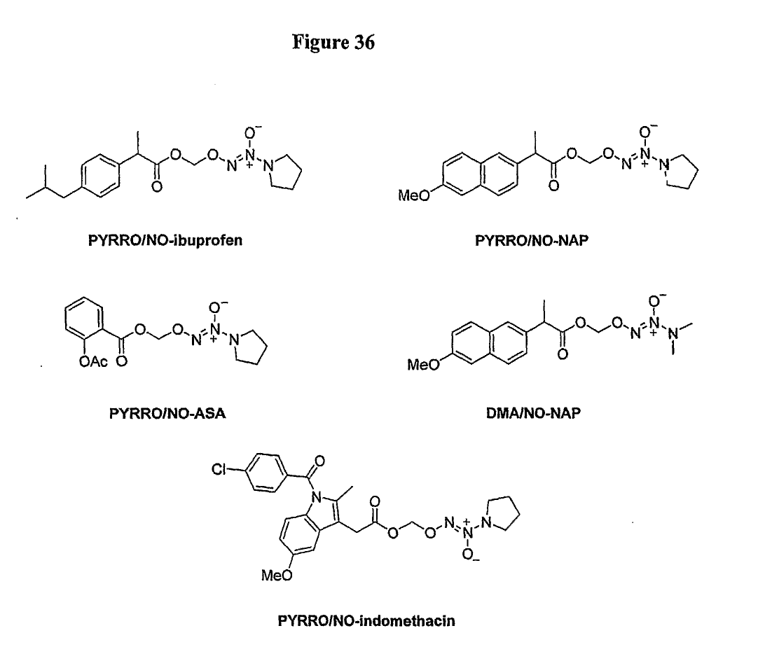

The output data are summarized in FIGS. 29-35 and Table 10. In general, the PBPK/PD model adequately predicts the plasma concentration of NAPROXEN (FIGS. 29 & 30) and NAPROXEN formed from AZD 3582 (FIGS. 31 & 32 and 34 & 35) The predicted values are within a two-fold range of the reported data. Consistent with the data reported in the literature (Fagerholm et al., 2005 and Fagerholm & Bjornsson, 2005), the model also predicts extremely low AZD 3582 bioavailability. The cause for the extensive first-pass effect is due to its extensive hydrolysis in the intestine and the liver. Both organs have significant contributions to its first-pass removal in rat and human (FIGS. 33 and 36). Since the fate of the metabolites which carry the NO donor is not known, it is difficult to predict where NO is being generated. It should be noted that rat and human are different in terms of first-pass removal of AZD 3582. Liver first-pass is dominant in rat; whereas intestinal first-pass is dominant in human. This example shows the importance of using in vitro tests instead of using in silico prediction when it comes to metabolism estimation. As a general rule, the rate of metabolism in rat is faster than that in human; however, we do see several differences in the present studies.

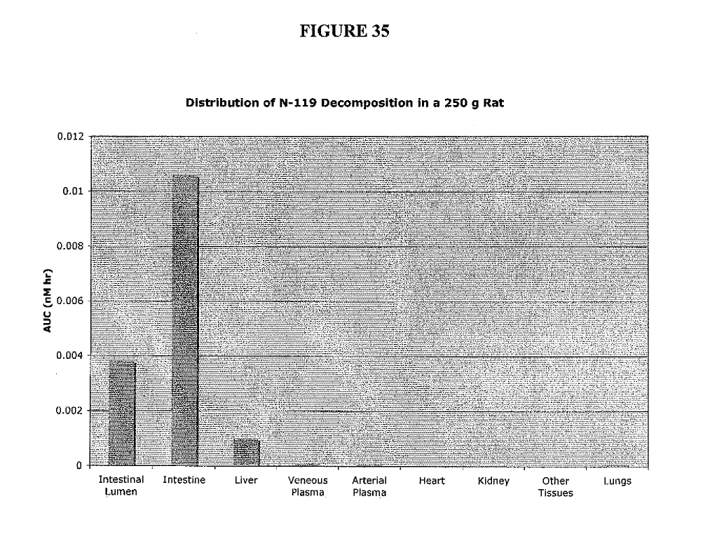

FIG. 35 shows the AUC of the diazeniumdiolate in the lumen, gastrointestinal tissue, liver, heart, kidneys and the rest of the body after oral N-119 administration. Since N-119 is not stable in the intestinal lumen and the rate of hydrolysis is high in rat intestinal microsomes (Table 9), this prodrug candidate released most of its nitric oxide before it enters the liver. Is estimated that systematic exposure of nitric oxide will be at a minimum. The results of this simulation are consistent with that the relatively low plasma nitrate level after the administration of PYRO/NO-naproxen. The plasma nitrate level is not that much higher than that of AZD 3582, although the NO generating capacity of PYRO/NO-naproxen is 13 times higher than that of AZD 3582 in vitro (Knaus et al., 2005). This set of data is consistent with the speculation that the set of candidates generated by Knaus et al. (2005) is not appropriate for further development, despite observable effects in the heart and kidneys.

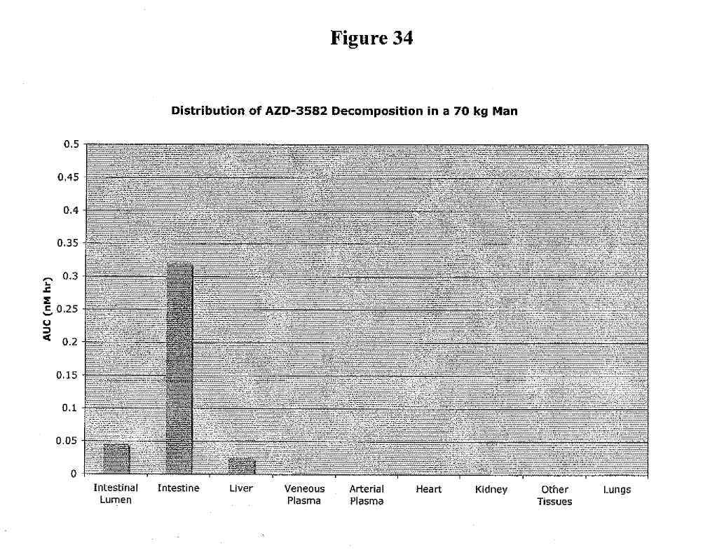

The pharmacokinetic behavior of AZD 3582 is similar to that of PYRO/NO-NAPROXEN except that AZD 3582 is more stable in the intestinal lumen and intestinal microsomes (Table 9). The release profile of NAPROXEN (FIGS. 33-34), after the administration of AZD 3582, suggests that the nitric oxide donor was also released in the first-pass organs. In the absence of measurable nitric oxide donor and metabolite data, it is not possible to estimate when NO was being generated. The in vivo study listed in Example 1 showed that the in vivo plasma nitrate levels were not high enough to trigger any observable effects in heart and kidneys (FIGS. 9-12). It is deduced that there is not enough NO being generated in the systemic circulation. This could be due to: (1) too much NO is being generated in the intestinal lumen, intestine and liver; and/or (2) the NO generating capacity is too low.

TABLE 10
Pharmacokinetic Parameters Estimated by the Model Using in vitro
and in silico Inputs Listed in Tables 8 and 9.

| | |
|---|---|
| | AZD3582 |
| Naproxen | Naproxen |

| PK Parameters | Rat | Human | Rat | Human |
|---|---|---|---|---|
| Dose, mg | 0.75 | 300 | 1.3 | 375 |
| V_{d}, L/kg | 0.09 | 0.12 | 0.09 | 0.12 |
| t_{1/2, hr} | 7.8 | 17.5 | 7.8 | 17.5 |
| Cₘₐₓ, μM | 50 | 125 | 55.2 | 83 |
| Tₘₛₐ, hr | 0.55 | 2.25 | 0.74 | 2.79 |

### EXAMPLE 5

### Developing Candidate Compounds

The present invention also provides a process of developing and improving the pipeline of compounds such as the described NO—NSAIDs using all of the elements described in the aforementioned examples.

The process begins with several prototype compounds with some of the desired characteristics, e.g. DMA/NO-NAPROXEN and PYRO/NO-NAP based on the Knaus (2005) chemistry. The simulations results of DMA/NO-NAPROXEN were similar to that of PYRO/NO-NAPROXEN. For the sake of simplicity, the results of PYRO/NO-NAPROXEN are shown above in Example 4.

The evaluations described in Example 4 show that the pharmacokinetic model described in this invention is capable of identifying imperfections of potential candidates. The candidates designed by Knaus et al. (2005) (FIG. 36) have the advantage of releasing a higher quantity of NO. However, these candidates lack specificities in NO delivery in terms of targeting internal organs such as heart and kidneys.

It becomes obvious from these simulations that an ideal candidate should have an optimal log P value at physiological pH. More importantly, the release of nitric oxide and NSAID should have certain degree of specificity. For example, it would be desirable to have a significant dose of NSAID released after prodrug administration, such that the antiiflammatory action will take effect soon. However, the NO donor should be less labile during the first-pass after the prodrug administration.

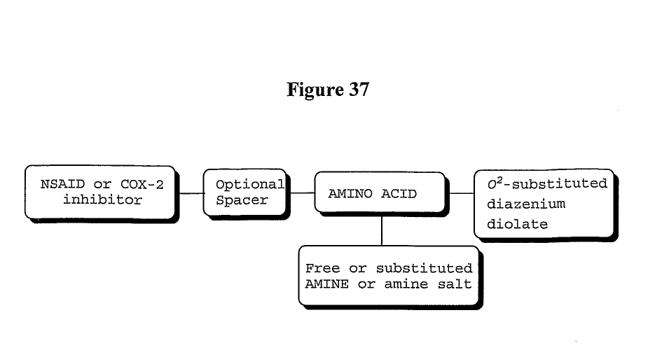

Based on the simulation results, the present invention describes a modular chemical library (FIG. 37) which can be used to systematically control the physical properties and kinetics of a NO prodrug so that systemic delivery of NO can be achieved. The goal is to deliver an optimal dose of NO to the gastrointestinal tract, heart and kidneys so that the potential side effects of NSAIDs can be mitigated.

Examples of NO-NSAID prodrugs containing an amino acid have been reported (Ranatunge et al., 2006; Kartasasmita et al., 2002; Andersson et al., 2004; Gilmer et al., 2002; Almirante et al., 2006; Benedini et al., 2000; Bolla et al., 2005; Rivolta et al., 2005; Del Soldato, 2002a, 2002b, 2003, 2004a and 2004b). These are limited to examples employing nitrooxyalkyl functionality as the source of NO. The rate of NO release from these prodrugs (or degradation products thereof) is determined by the rate of nitrooxyalkyl reduction, which occurs via multiple pathways (Fung, 2004; Carini et al., 2002; Gao et al., 2005; Satyum, 2006) and is therefore difficult to control.

In the present invention, the release rate of NO from the prodrug can be systematically controlled. The mechanism of NO release from the O²-substituted diazeniumdiolate occurs in two distinct steps. Enzymatic release of the diazeniumdiolate from the prodrug gives an O²-unsubstituted diazeniumdiolate which subsequently undergoes rapid decomposition under physiological conditions to release NO. If the correct O²-substituted diazeniumdiolate is used, the NO-NSAID candidate will be stable towards physiological pH. If the release rate of diazeniumdiolate from the candidate compound via enzymatic hydrolysis (t_{1/2}=minutes to hours) exceeds the half life of the released unsubstituted diazeniumdiolate to a sufficient degree (examples include, but are not limited to, PROL-NO (t_{1/2} 2 s, Keefer, 2005), PYRRO-NO (t_{1/2} 3 s, Saavedra, 2000)), it can be considered that enzymatic hydrolysis of the ester linkage between the amino acid and diazeniumdiolate is the rate determining step for NO release.

The rate of enzymatic release can be controlled by a number of factors. In the NO-NSAID candidates developed by Knaus et al. (2005) (FIG. 8), the O²-substituted diazeniumdiolate is directly attached to the NSAID. Enzymatic cleavage results in concomitant release of both the NSAID and O²-unsubstituted diazeniumdiolate. The rate of enzymatic hydrolysis and therefore the release rate of NO and NSAID is directly controlled by the choice of the diazeniumdiolate. One limitation of this approach is that in addition to altering the rate of enzymatic hydrolysis, altering the diazeniumdiolate can affect both the half-life (t_{1/2}) and efficiency of NO generation. Furthermore, the toxicity/metabolic profile will change because different secondary amines will be generated during NO release. Although the list of potential diazeniumdiolates derived from secondary amines and amino acids is large, the number of biologically viable derivatives that can be used in a prodrug is limited due to the toxicity of many amines and/or their metabolic products (Mattioni et al., 2003; Myers et al., 1997). It is therefore desirable to be able to control the rate of release of a specific diazeniumdiolate derivative with well characterized kinetic and toxicological parameters when developing NO-NSAID candidates.

In the present invention, the NSAID or COX-2 inhibitor and the O²-substituted diazeniumdiolate are independently attached to a central amino acid via functionality (typically ester, thioester, amide or sulfonamide) which is susceptible to enzymatic hydrolysis or cleavage. This permits the enzymatic release of the NSAID and diazeniumdiolate to occur at different rates. Control of the absolute and relative release rates of NO (resulting from the release of the diazeniumdiolate) and a specific NSAID can be controlled by modifying the modules of the structure (FIG. 37), specifically the amino acid, the amino acid nitrogen substituent, and/or the spacer connecting the NSAID to the amino acid. Although the choice of diazeniumdiolate will modify the rate of enzymatic release, the enzymatic release rate can be changed by altering the other modules while keeping the diazeniumdiolate constant. This invention permits generation of new candidates with different kinetic, physical, pharmacodynamic and pharmacokinetics properties without the need to alter the NSAID or diazeniumdiolate.

Enzymatic degradation of the modular structure of the present invention is designed to produce NO, a secondary amine, formaldehyde, an N-substituted amino acid and the NSAID. N-substituted amino acids can be considered as either prodrugs of the corresponding parent amino acid, (Pitman, 1981) or additional therapeutic agents (Chandran, 2005; Yu et al., 2006). Examples of suitable N-substituents include (but are not limited to) amides (Crankshaw et al., 2002), carbamates (Hansen et al., 1992) and α-hydroxy or α-acyloxymethyls (Bundgaard et al., 1987).

### EXAMPLE 6

### Controlling the Release Rate of Diazeniumdiolate from the NO-NSAID Prodrug

Three examples of the modular library (1-3) were synthesized based on three common modules; NAPROXEN as the NSAID, hydroxyproline as the amino acid and DMA diazeniumdiolate. They varied only in the nitrogen substituent of the amino acid, i.e. free amine (1), acetyl (2) and pivaloyl (3) groups. Their chemical stability was evaluated in phosphate buffer at different pH's over a 30 minute period (Table 11). The free amine 1 underwent rapid degradation over the pH range 2.5-7.0 to release the unsubstituted diazeniumdiolate (not shown) and the NSAID-amino acid 4. The N-acetyl derivative 2 was determined to undergo a much slower rate of decomposition to generate the unsubstituted diazeniumdiolate (not shown) and NSAID-amino acid 5 at pH 7.0. The prodrug 2 was found to be stable at pH 2.5-5.0 over a 30 minute period. A pivaloyl amide 3 was observed to be stable across the entire pH range 2.5-7.0.

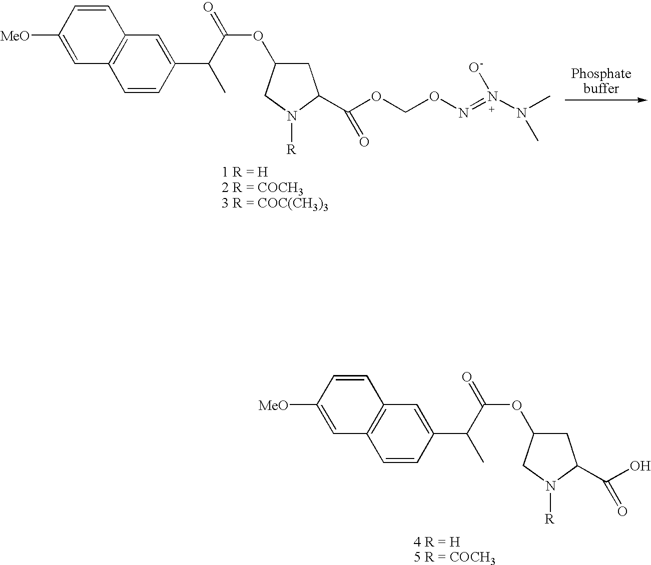

TABLE 11
Loss of Diazeniumdiolate With Phosphate Buffer at Various pH's

| | Stability (phosphate buffer, 30 min) | | |
|---|---|---|---|
| Compound | pH 2.5 | pH 5.0 | pH 7.0 |
| 1 | x | x | x |
| 2 | ✓ | ✓ | x |
| 3 | ✓ | ✓ | ✓ |

The sensitivity of the prodrug 3 towards enzymatic hydrolysis was evaluated by LC-MS. Liver and intestinal microsomal preparations were used. It was found that cleavage of the unsubstituted diazeniumdiolate resulting in the formation of the NSAID-amino acid 6 was rapid in both the liver (complete hydrolysis after 2 hours) and intestinal (50% conversion after 2 hours) preparations. However, these rates were slower than the enzymatic hydrolysis rates determined for the conversion of 1→4 and 2→5 (complete hydrolysis observed in 10 and 30 minutes respectively in liver microsomal preparations).

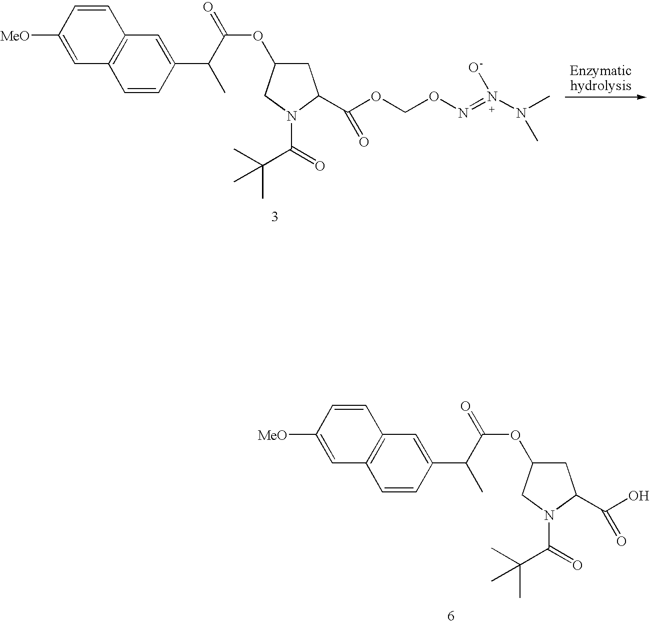

Enzymatic hydrolysis of the ester linking NAPROXEN to the amino acid was found to be slow in all cases. After 2 hours, a 2% release of naproxen was observed in liver microsomes.

A further embodiment of this invention is the recognition that the use of two non-equivalent esters to independently release the NSAID and diazeniumdiolate may proceed via the action of specific esterases or other enzyme classes. As the distribution of esterases varies throughout human tissues and organs, it is possible for specific enzymes to release either the NSAID and/or the diazeniumdiolate selectively at a specific target tissue or organ.

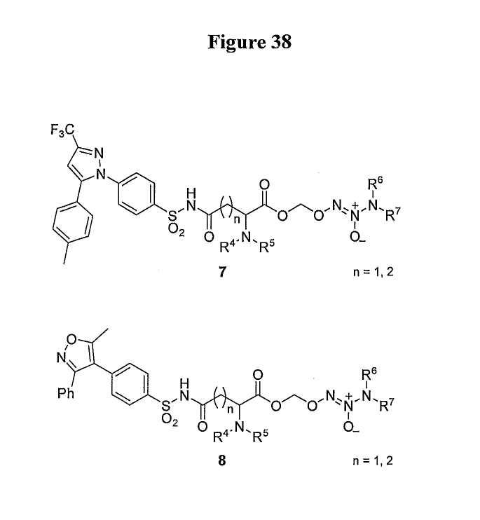

A limitation of the NO-NSAIDS developed by Knaus et al. (2005) is the exclusive use of NSAIDs that contain carboxylic acids (FIG. 36). A similar limitation applies to the attachment of the NSAID IBUPROFEN via an amide linkage to the distal nitrogen of a piperazine diazeniumdiolate as reported by Saavendra et al. (1999). A further embodiment of this invention is the ability to attach other functionalized drugs or linker units (including but not limited to alcohols, thiols, amines, sulfonamides) to the amino acid module when the parent amino acid is a diacid such as aspartic (n=1) or glutamic acid (n=2). This principle is demonstrated by the diazeniumdiolate prodrugs of CELECOXIB and VALDECOXIB, potent and selective COX-2 inhibitors (FIG. 38). In these examples, the drug (CELECOXIB or VALDECOXIB) is attached to the amino acid of the prodrug via its sulfonamide functionality to give structures 7 and 8 respectively.

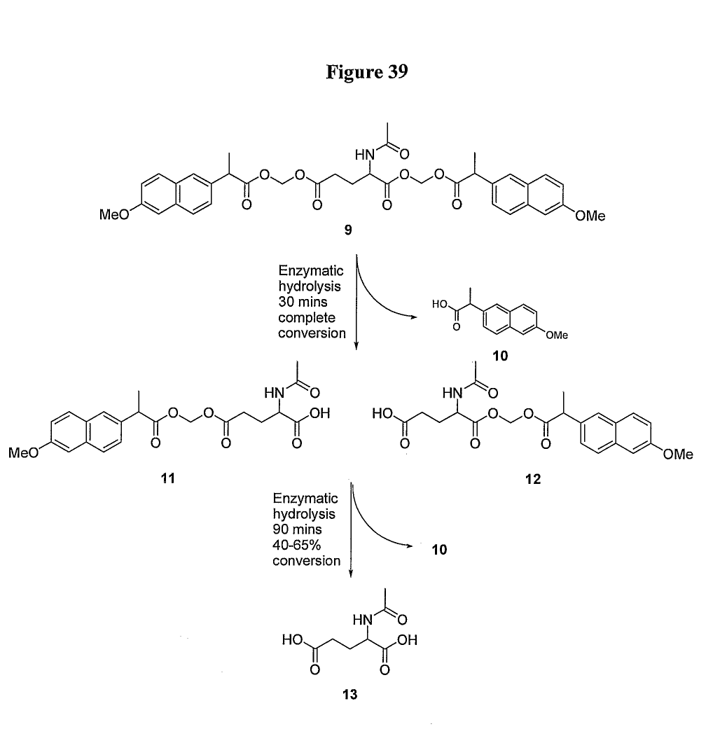

A further embodiment of this invention is the recognition that cleavage of either the NSAID/linker or the diazeniumdiolate from a diacid amino acid module (including but not limited to aspartic and glutamic acid) results in a pronounced reduction in the rate of enzymatic hydrolysis of the remaining NSAID/linker or diazeniumdiolate. This is exemplified by the di-NAPROXEN prodrug (NAP-AA-NAP) 9 (FIG. 39). Initial enzymatic cleavage of both esters occurs rapidly and non-selectively (complete hydrolysis after 30 minutes in liver microsomes) to give a mixture consisting predominantly of NAPROXEN (AA-NAP) prodrugs 11 and 12. Cleavage of the second linker released a further molecule of NAPROXEN from the AA-NAP's 11 and 12. This however proceeds at a much slower rate (40-65% conversion to 13 after 90 minutes).

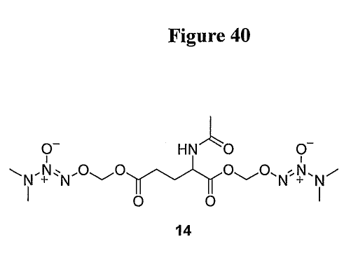

Studies on di-DMA-diazeniumdiolate N-acyl glutamic acid (DMA/NO-AA-DMA/NO) 14 (FIG. 40) have shown that an analogous change of hydrolysis rate also occurs when esters link diazeniumdiolates to glutamic and aspartic acid amino acids.

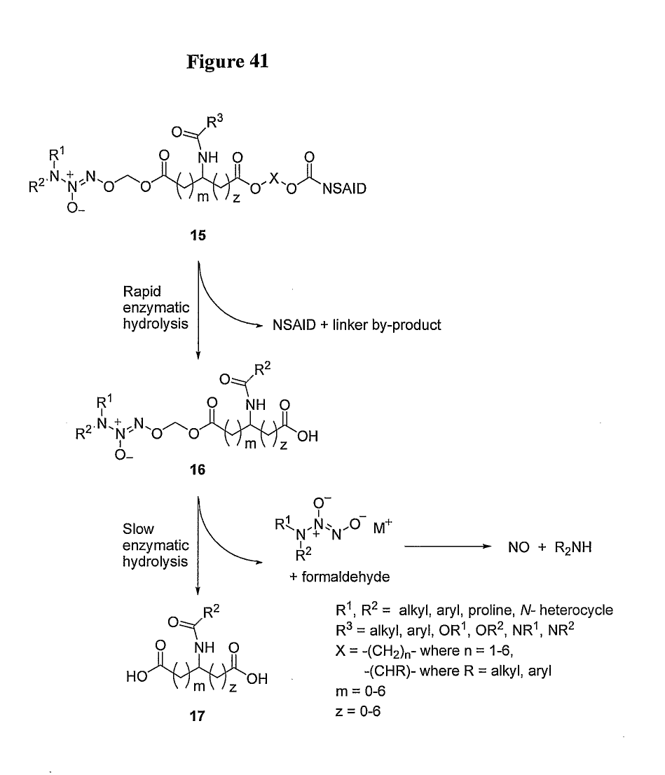

This principle can be further extended to a diazeniumdiolate based NO-NSAID prodrugs (NO-AA-NSAID) 15 (FIG. 41). If the two ester linkages for the NSAID and diazeniumdiolate moieties are initially hydrolyzed at different rates (for example, the NSAID is initially cleaved more rapidly than the diazeniumdiolate), the NSAID will be predominantly released first from 15. This will generate a mono acidic diazeniumdiolate prodrug ester (NO-AA) 16 and the free NSAID (or biologically relevant salts thereof). The (NO-AA) 16 would subsequently undergo slower enzymatic release of the diazeniumdiolate than is the case for the NO-AA-NSAID 15, therefore providing a slow release of NO. conversely, if the diazeniumdiolate was initially cleaved faster from the NO-AA-NSAID, then there will be fast generation of diazeniumdiolate (and therefore NO) with slow release of the NSAID

### EXAMPLE 7

### Differential Release of the NSAID and the Diazenium-Diolate from NO-AA-NSAID

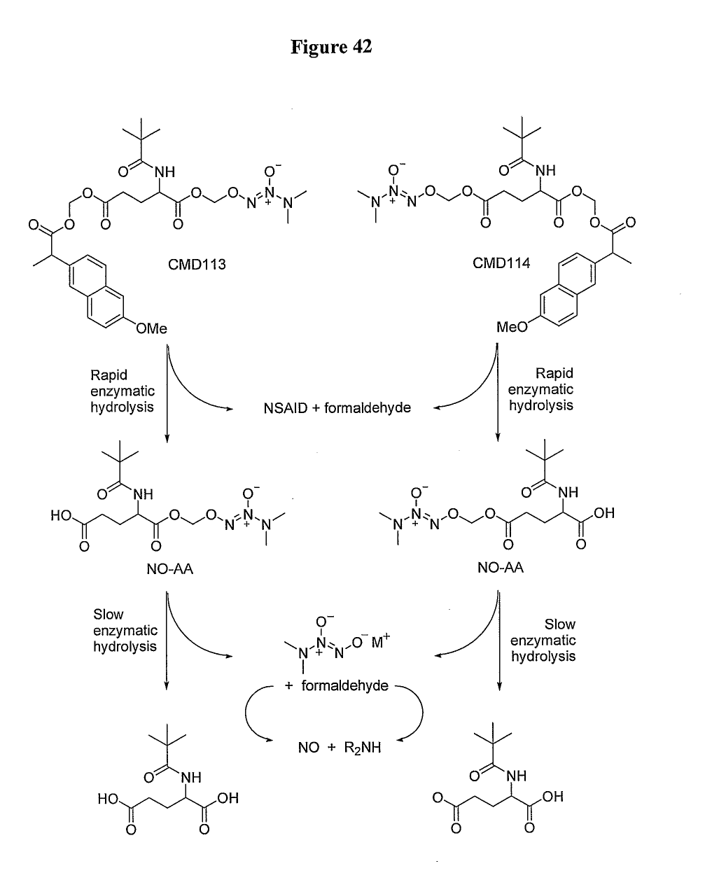

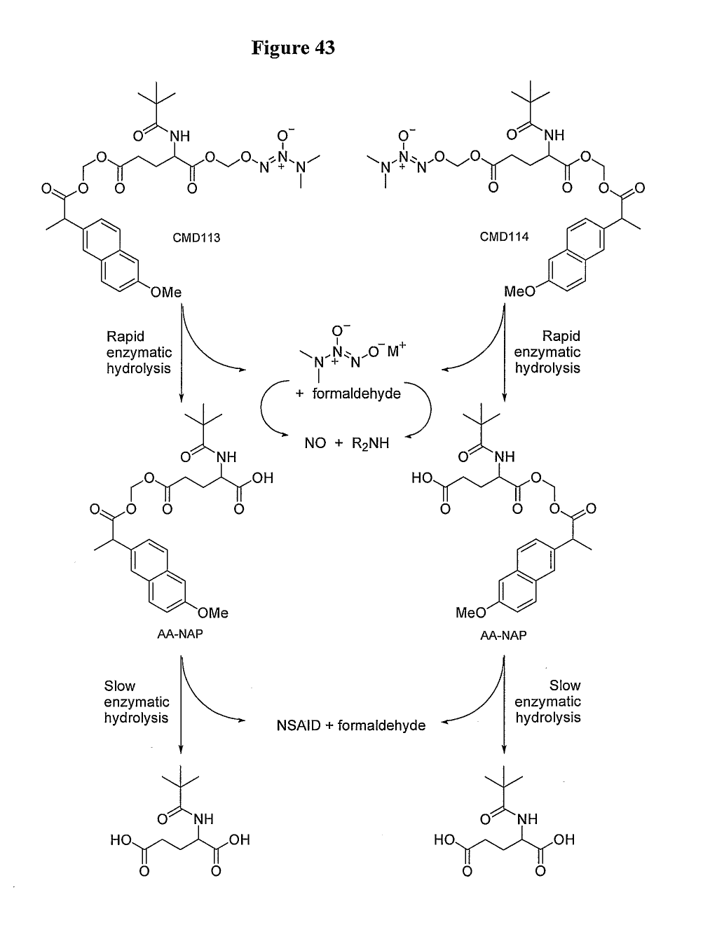

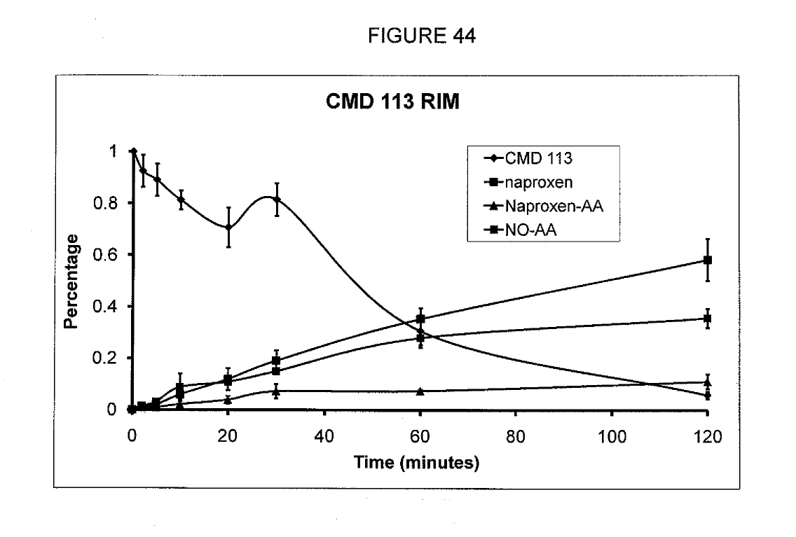

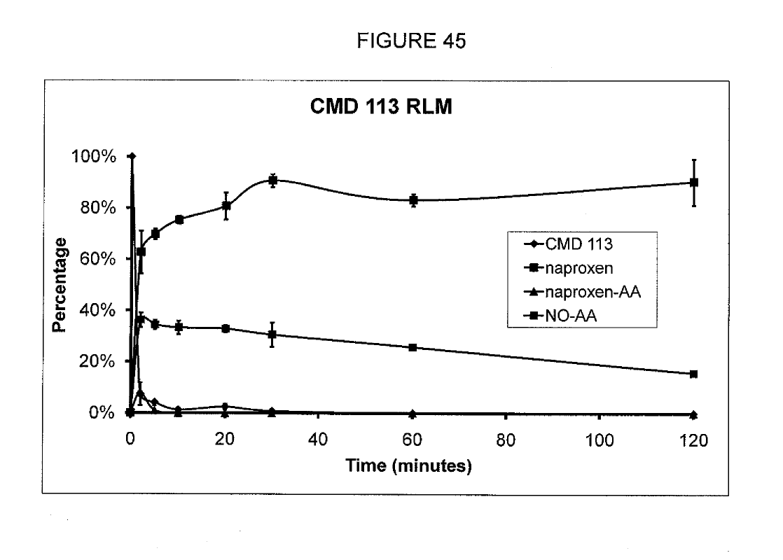

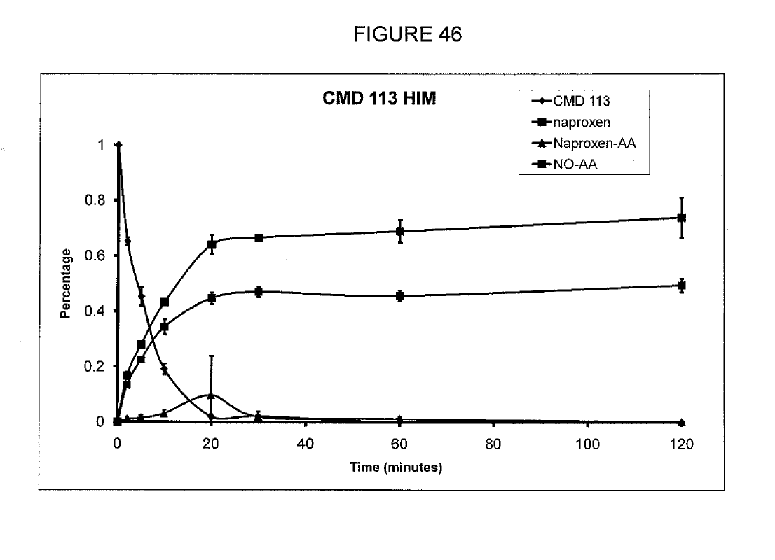

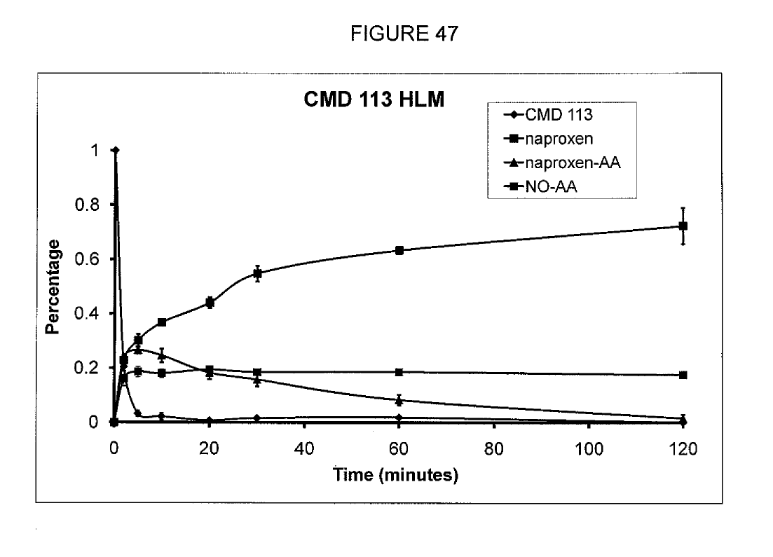

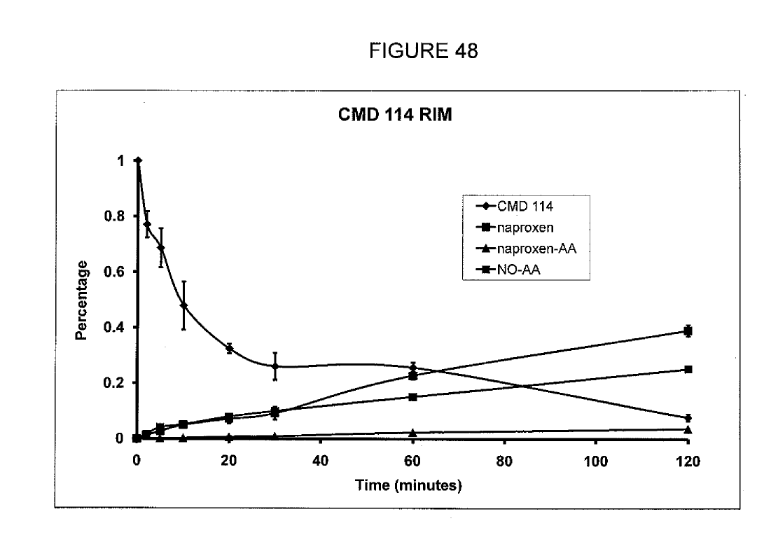

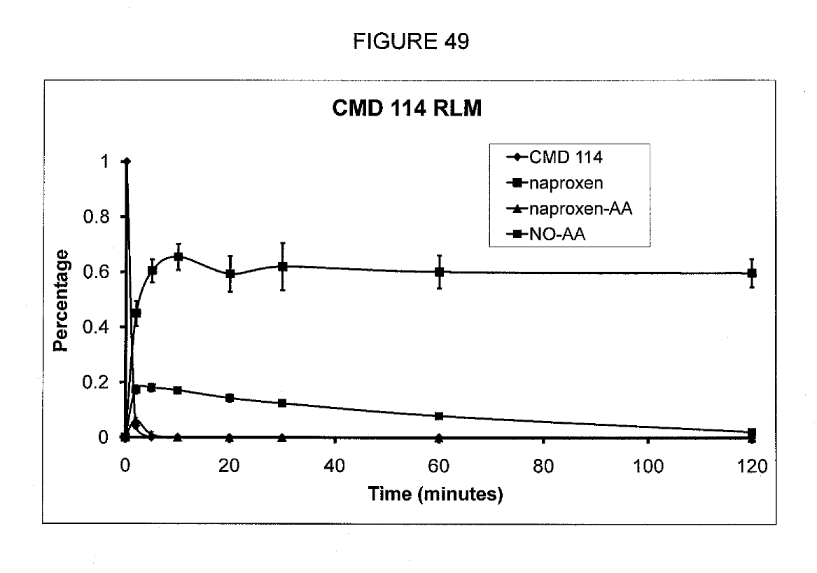

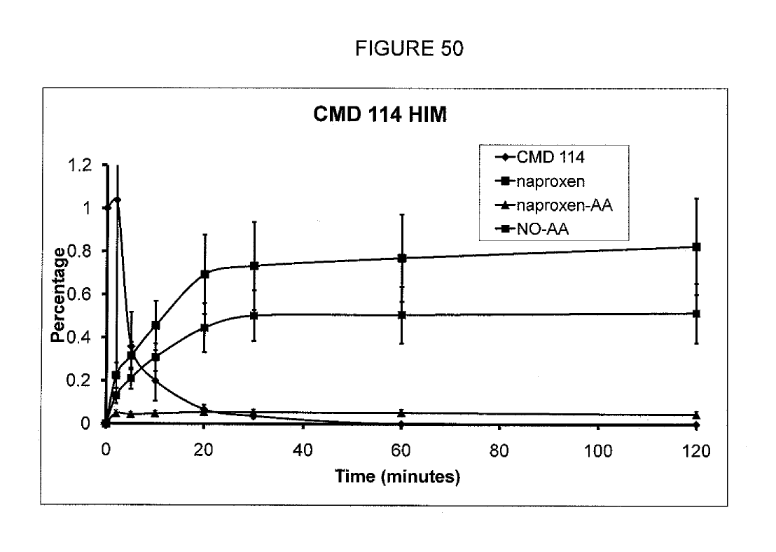

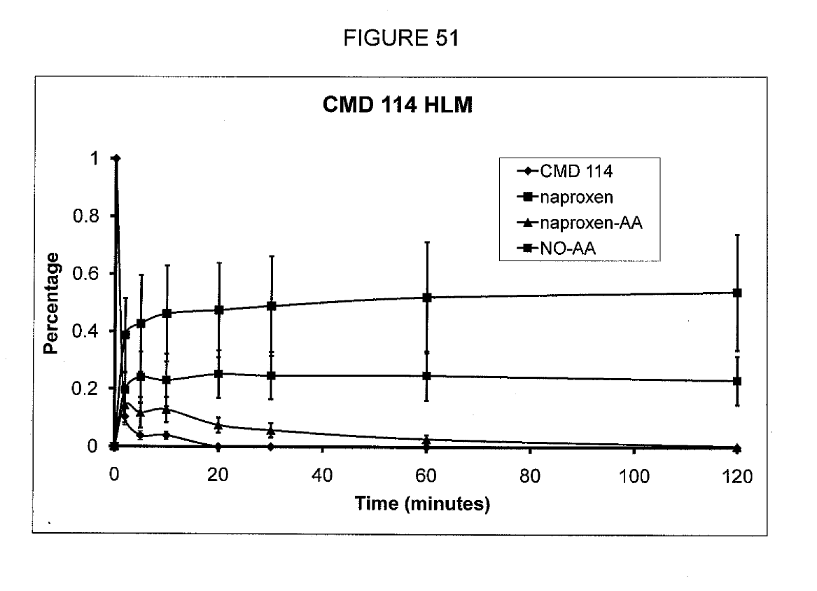

This principle of differential hydrolysis rates has been demonstrated for NO-AA-NSAIDS CMD113 and CMD114 (FIGS. 42-43). Exposure of both CMD113 and CMD114 to various enzymatic preparations (FIGS. 44-51) resulted in rapid initial loss of the prodrug candidate to give NAPROXEN and the NO-AA (FIG. 42) and slower competitive release of the dimethylamino diazeniumdiolate to give the AA-NAP (FIG. 43). Subsequent enzymatic hydrolysis of the remaining ester in both the NO-AA (FIG. 42) and AA-NAP (FIG. 43) proceeded at a much slower rate than the initial hydrolysis to give N-pivaloyl glutamic acid.

It is important to note that the NO-AAs formed from CMD 113 and CMD 114 have several features in common. The first one is that both compounds are relatively stable in both intestinal and liver microsomes of human and rats (FIGS. 44-51). The second one is that both of these compounds hydrolyzed in the rat plasma at a relatively fast rate (Tables 12 & 13). It is clear that NO-AAs, with similar structures, may be stable after they are released from their respective prodrug moiety in intestinal and liver; but these species will be able to release NOD and subsequently NO in plasma. The difference in the response to intestinal and liver microsomes vs. plasma is an important feature in the design of NO-AA. Ideally, the structure of a potential NO-AA should be susceptible to all esterases, with optimal release rates.

TABLE 12
Input Parameters for the Simulation of CMD 113

| CMD 113 | Rat | Human | Source |
|---|---|---|---|
| Intestinal microsomal activity, | 2 | 4813 | In-house data |
| mL/min/organ | | | |
| Liver microsomal activity, | 6545 | 434763 | In-house data |
| mL/min/organ | | | |
| Intestinal molar conversion to | 62 | 75 | In-house data |
| naproxen, % | | | |
| Liver molar conversion to | 90 | 72 | In-house data |
| naproxen, % | | | |
| gastric decay rate constant, /mint | 0 | 0 | In-house data |
| intestinal lumen decay rate | 0.2 | 0.2 | In-house data |
| constant,/min | | | |
| plasma decay rate constant, /min | 1.2 | — | In-house data |
| NO-AA half-life in plasma, min. | 9.2 | — | In-house data |

TABLE 13
Input Parameters for the Simulation of CMD 114

| CMD 114 | Rat | Human | Source |
|---|---|---|---|
| Intestinal microsomal activity, | 9 | 5006 | In-house data |
| mL/min/organ | | | |
| Liver microsomal activity, | 7667 | 721224 | In-house data |
| mL/min/organ | | | |
| Intestinal molar conversion to | 42 | 78 | In-house data |
| naproxen, % | | | |
| Liver molar conversion to | 60 | 50 | In-house data |
| naproxen, % | | | |
| gastric decay rate constant, /mint | 0 | 0 | In-house data |
| intestinal lumen decay rate | 0.67 | 0.67 | In-house data |
| constant,/min | | | |
| plasma decay rate constant, /min | — | — | In-house data |
| NO-AA half-life in plasma, min. | 3.8 | — | In-house data |

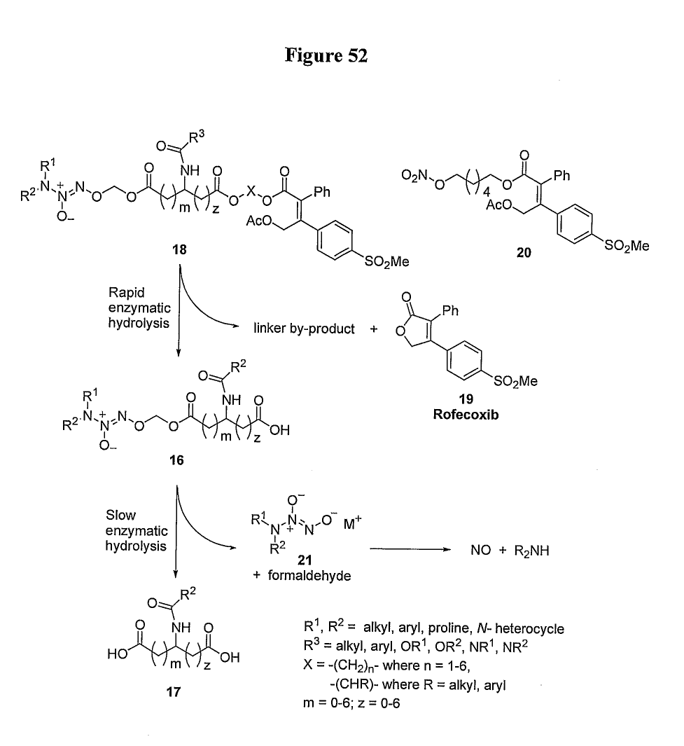

This principle can be extended to include COX-2 inhibitors (including but not limited to ROFECOXIB). Prodrugs of COX-2 inhibitors such as ROFECOXIB and some COX-2-inhibitors contain a carboxylic acid or alcoholic functional handles (for examples see Black et al., 1997, 1998a, 1998b, 1999), which can be used to attach the molecule to the modular scaffold described herein. In such cases, the COX-2 inhibitor prodrug (such as that shown in 18 based on a known ROFECOXIB prodrug 20 (Engelhardt et al., 2006) will be released rapidly (as described previously for the analogous NSAID derivatives), resulting in the same NO-AA 16 (FIG. 52). As before, hydrolysis of the NO-AA 16 to release the diazeniumdiolate 21 (and therefore NO) will be significantly slower than the initial hydrolysis step. This will provide for rapid release of the COX-2 inhibitor (or prodrug thereof) and slow release of the diazenium diolate (and therefore NO).

### EXAMPLE 8

### General Methods for the Preparation of NONOate-Amino Acid-NAPROXEN Prodrugs 26

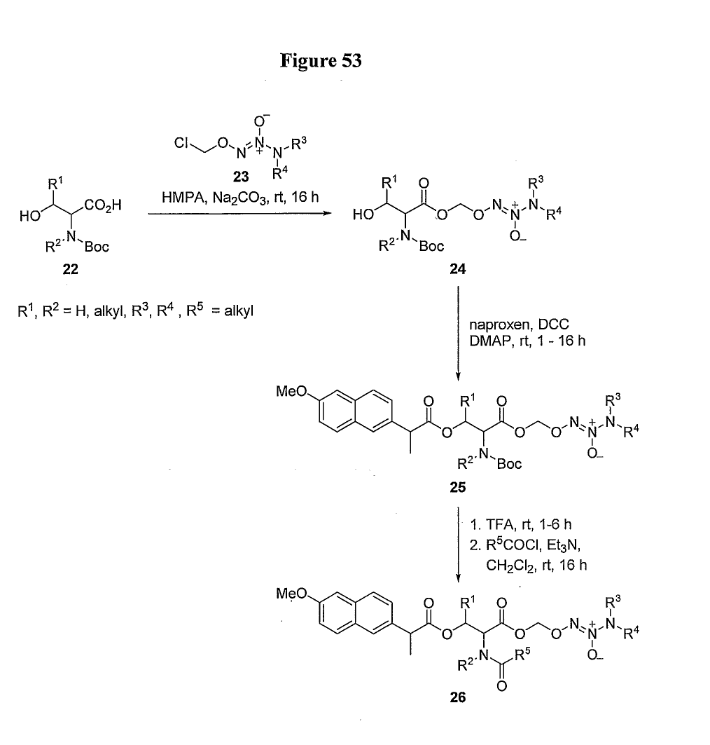

### N-Boc Protected NO-AA's 24 (FIG. 53)

A solution of the chloride 23 [(11.0 mmol), cf. Knaus et al. (2005)], in hexamethylphosphorus triamide (HMPA) (5 mL) was added to a suspension of the N-Boc amino acid 22 (9.13 mmol), (Engelhardt et al., 2006) and Na₂CO₃ (9.13 mmol) in HMPA (5 mL) at room temperature (rt) and the resulting mixture was stirred overnight. Water was then added to the mixture and the resulting aqueous layer was extracted with ethyl acetate (EtOAc) (×5). The organic fractions were collected, dried (Na₂SO₄ or MgSO₄) and concentrated in vacuo. The residue was purified by flash chromatography (silica gel) eluting typically with hexane/EtOAc to give 24.

### N-Boc Protected NO-AA-NAP's 25 (FIG. 53)

A solution of 24 (1.0 mmol), naproxen (1 mmol) dicyclohexylcarbodiimde (DCC) (1.0 mmol) and 4-(dimethylamino)pyridine (DMAP) (0.1 mmol) in anhydrous CH₂Cl₂ (10 mL) was stirred at rt for a period of 1 h—overnight [(the reaction was monitored by thin layer chromatography (TLC)]. The resulting white precipitate was removed by filtration and the filtrate was concentrated in vacuo. The residue was purified by flash chromatography (silica gel) eluting typically with hexane/EtOAc to give 25.

### N-Acetyl NO-AA-NAP's (R⁵=Me) 26 (FIG. 53)

25 (0.1 mmol) was dissolved in trifluoroacetic acid (TFA) (1 mL) at rt and stirred for 1-6 h (reaction monitored by TLC). The resulting mixture was concentrated in vacuo. The residue was taken up into acetic acid (AcOH) (1 mL) and acetic anhydride (Ac₂O) (0.18 mL) was added dropwise with stirring at rt. The resulting mixture was stirred at rt overnight. The reaction mixture was then concentrated in vacuo and the residue was purified by flash chromatography (silica gel) eluting typically with EtOAc/CHCl₃ to give 26.

### N-Pivaloyl NO-AA-NAP's (R⁵═CMe₃) 26 (FIG. 53)

25 (1 mmol) was dissolved in TFA (5 mL) at rt and stirred for 1-6 h (reaction monitored by TLC). The resulting mixture was concentrated in vacuo. The residue was taken up into CH₂Cl₂ (5 mL) and pivaloyl chloride (PivCl) (0.17 mL) followed by Et₃N (0.32 mL) were added dropwise with stirring at rt. The resulting mixture was stirred at rt overnight. The reaction mixture was then concentrated in vacuo and the residue was purified by flash chromatography (silica gel) eluting typically with EtOAc/hexane to give 26.

### EXAMPLE 9

### General Methods for the Preparation of N-Ac NAP-Glu-NAP 9

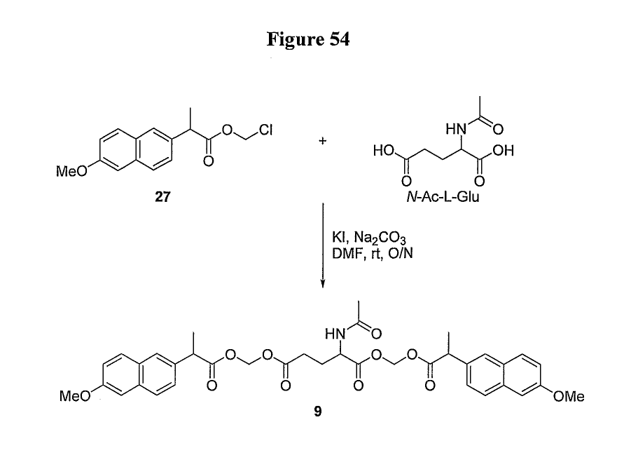

The method is shown in FIG. 54. A suspension of N-Ac-L-Glu (250 mg, 1.3 mmol), chloride 27 [(362 mg, 1.3 mmol) Phelan et al. (1989)], KI (50 mg, 0.3 mmol) and Na₂CO₃ (140 mg, 1.3 mmol) in anhydrous dimethylformamide (DMF) (10 mL) was stirred at rt overnight. The reaction mixture was then concentrated in vacuo and the resulting residue was then taken up into water. The pH was adjusted to 2 using 1N HCl and the aqueous layer was then extracted with EtOAc (×3). The organic fractions were collected, dried (Na₂SO₄) and concentrated in vacuo. The residue eluting initially with 1:1 hexane:EtOAc and then 2:1 EtOAc:hexane to give 9 as a white solid (132 mg, 30%).

### EXAMPLE 10

### General Methods for the Preparation of N-Ac DMA/NO-Glu-NO/DMA 14

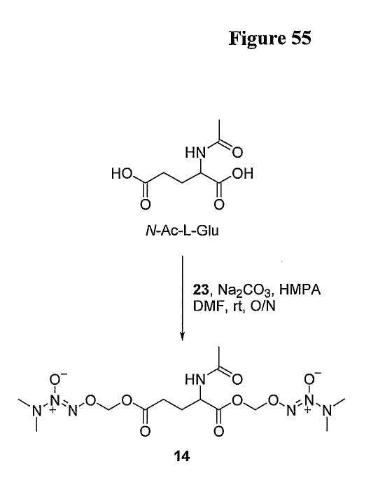

The method is shown in FIG. 55. A suspension of the DMA chloride 23 [(3.2 mmol), Knaus et al. (2005)], N-acetyl-L-glu (250 mg, 1.32 mmol) and Na₂CO₃ (280 mg, 2.64 mmol) in HMPA (3 mL) was stirred at rt overnight. Water was then added to the mixture and the resulting aqueous layer was extracted with EtOAc (×3). The organic fractions were collected, dried (Na₂SO₄) and concentrated in vacuo. The residue was purified by flash chromatography (silica gel) eluting with EtOAc to give 14 (239 mg, 43%).

### EXAMPLE 11

### General Methods for the Preparation of CMD113 and CMD114

### N-Boc-O-Benzyl Glu-NAP 28 and 29

A mixture of the Boc-L-Glu benzyl ester (1.36 mmol), chloride 27 [(1.36 mmol) Phelan et al. (1989)], KI (100 mg, 0.6 mmol) and Na₂CO₃ (150 mg, 1.36 mmol) in anhydrous DMF (10 mL) was stirred at rt overnight. The reaction mixture was then concentrated in vacuo and the resulting residue was then taken up into water. The aqueous layer was then extracted with EtOAc (×3). The organic fractions were collected, dried (Na₂SO₄) and concentrated in vacuo. The residue was purified by flash chromatography (silica gel) eluting typically with EtOAc/hexanes to give the title compounds.

### Deprotection of 1-Boc-O-Benzyl Glu-NAP 30 and 31

A suspension of the N-Boc-O-Benzyl Glu-NAP (0.79 mmol) and 5% Pd/C (50 mg) in EtOAc (50 mL) was stirred vigorously under an atmosphere of H₂ (1 atm) at rt 3-6 h (the reaction was monitored) by TLC). The mixture was then filtered through a pad of Celite and the filtrate was concentrated in vacuo to give the title compounds.

### Synthesis of N-Boc Protected DMA/NO-Glu-NAP 32 and 33

A suspension of the chloride 23 [(0.73 mmol), Knaus et al. (2005)], N-Boc-O-Benzyl Glu-NAP (0.49 mmol) and Na₂CO₃ (78 mg, 0.74 mmol) in HMPA (5 mL) was stirred at rt overnight. Water was then added to the mixture and the resulting aqueous layer was extracted with EtOAc (×3). The organic fractions were collected, dried (Na₂SO₄) and concentrated in vacuo. The residue was purified by flash chromatography (silica gel) typically eluting with EtOAc/hexane to give the title compounds.

### N-Pivaloyl Glutamic Acid-NONOates CMD113 and CMD114

N-Boc Protected DMA/NO-Glu-NAP (0.31 mmol) was dissolved in TFA (3 mL) at rt and stirred for 30 min-2 h (reaction monitored by TLC). The resulting mixture was concentrated in vacuo. The residue was taken up into CH₂Cl₂ (5 mL) and pivaloyl chloride (58 μL) followed by Et₃N (100 μL) were added dropwise with stirring at rt. The resulting mixture was stirred at rt overnight. The reaction mixture was then concentrated in vacuo and the residue was purified by flash chromatography (silica gel) eluting typically with EtOAc/hexane to give the title compounds.

### REFERENCES

Al Awwadi N A, Borrot-Bouttefroy A, Umar A, Saucier C, Segur M C, Garreau C, Canal M, Glories Y, Moore N Effect of Armagnac fractions on human platelet aggregation in vitro and on rat arteriovenous shunt thrombosis in vivo probably not related only to polyphenols. Thromb Res. 2006 Jun. 21; [Epub ahead of print].
Almirante N. Ferrario M, Ongini E: Process for preparing nitrooxy esters, nitrooxy thioesters, nitrooxy carbonates and nitrooxy thiocarbonates, intermediates useful in said process and preparation thereof: 2006. Int. Patent No: 2006/008196 A1.
Andersson J, Belli A, Cannata V, Hedberg M, Palmgren A, Schuldei S, Ström M, Villa M: Manufacturing process for NO-donating compounds such as NO-donating Diclofenac. 2004. Int. Patent No. WO 2004/026808 A1.
Bandarage et al. 2000; UK, Dong Q, Fang X, Garvey D S, Mercer G J, Richardson S K, Schroeder J D, Wang T: Nitrosated and nitrosylated nonsteroidal anti-inflammatory compounds, compositions and methods of use. Int. Patent No. WO 00/25776 A1.
Bandarage U K, Fang X, Garvey D S, Letts L G, Schroeder J D, Tam S W: Nitrosated and nitrosylated cyclooxygenase-2 inhibitors, compositions and methods of use. 2003. U.S. Pat. No. 6,649,629 B2.
Bandarage U K, Earl R A, Ezawa M, Fang X, Garvey D S, Khanpure S P, Ranatunge R R, Richardson S K, Schroeder J D: Cyclooxygenase-2 selective inhibitors, compositions and methods of use. Int. Patent No. WO 2004/002409 A2
Beaumont K, Webster R, Gardner I, Dack K: Design of ester prodrugs to enhance oral absorption of poorly permeable compounds: challenges to the discovery scientist. Current Drug Metabolism 2003, 4: 461-485.
Benedini F, Del Soldato P: Nitrooxy derivatives giving anti-inflammatory, analgesic and antithrombotic activity. 2000. Int. Patent No. WO 00/51988 A1.
Bernareggi A, Rowland M: Physiologic Modeling of Cyclosporine Kinetics in Rat and Man. J. Pharmacokin. Biopharm. 1991, 19(1): 21-49.
Black C, Leger S, Prasit P, Wang Z, Han Y, Hughes G: 3,4-Diaryl-2-hydroxy-2,5-dihydrofurans as prodrugs to COX-2 inhibitors. 1997. U.S. Pat. No. 5,698,584.
Black C, Girard M, Guay D, Wang Z: Diphenyl stilbenes as prodrugs to COX-2 inhibitors. 1998a. U.S. Pat. No. 5,733,909.
Black C, Grimm E, Leger S, Hughes G, Prasit P, Wang Z: Alkylated styrenes as prodrugs to COX-2 inhibitors. 1998b. U.S. Pat. No. 5,789,413.
Black C, Hughes G, Grimm E, Leger S, Prasit P, Wang Z: Alkylated styrenes as prodrugs to COX-2 inhibitors. 1999. U.S. Pat. No. 5,925,631.
Bolla M, Santus G, Del Soldato P: Nitrosylated analgesic and/or anti-inflammatory drugs having antiviral activity. 2005. Int. Patent No. WO 2005/030224 A1.
Borna C, Lazarowski E, van Heusden C, Ohlin H, Erlinge D: Resistance to aspirin is increased by ST-elevation myocardial infarction and correlates with adenosine diphosphate levels. Thromb. J. 2005, 26: 3:10.
Bowalgaha K, Miners J O: The glucuronidation of mycophenolic acid by human liver, kidney and jejunum microsomes. Br. J. Clin. Pharmacol. 2001, 52: 605-609.
Bresalier R S, Sandler R S, Quan H, Bolognese J A, Oxenius B, Horgan K, Lines C, Riddell R, Morton D, Lanas A, Konstam M A, Baron J A; Adenomatous Polyp Prevention on Vioxx (APPROVe) Trial Investigators: Cardiovascular events associated with rofecoxib in a colorectal adenoma chemoprevention trial. N. Engl. J. Med. 2005, 352(11):1092-102.
Brooks P M, Day R O: Nonsteroidal anti-inflammatory drugs-differences and similarities. N. Engl. J. Med. 1991, 324: 1716-1725.
Brown R P, Delp M D, Lindstedt S L, Rhomberg L R, Beliles R P: Physiological Parameter Values for Physiologically based Pharmacokinetic Studies, Toxicol. Ind. Health 1997, 13(4) 407-484.
Bundgaard H. Buur A: Prodrugs as drig delivery systems. 65. Hydrolysis of α-hydroxy- and α-acyloxy-N-benzoylglycine derivatives and implications for the design of prodrugs of NH-acidic compounds. Int. J. Pharm. 1987, 37: 185-94.
Buttar N S, Wang K K: The ‘aspirin’ of the new millennium: cyclooxygenase-2 inhibitors. Mayo Clin. Proc. 2000, 75: 1027-38.
Cannon G W, Caldwell J R, Holt P, McLean B, Seidenberg B, Bolognese J, Ehrich E, Mukhopadhyay S, Daniels B: Rofecoxib, a specific inhibitor of cyclooxygenase 2, with clinical efficacy comparable with that of diclofenac sodium: results of a one-year, randomized, clinical trial in patients with osteoarthritis of the knee and hip. Rofecoxib Phase III Protocol 035 Study Group. Arthritis Rheum. 2000, 43(5): 978-87.
Carini M, Aldini G, Orioli M, Facino R M: In vitro metabolism of a nitroderivative of acetylsalicylic acid (NCX4016) by rat liver: LC and LC-MS studies. J. Pharm. Biomed. Anal. 2002, 29: 1061-71
Cathella-Lawson F, Reilly M P, Kapoor S C: Cyclooxygenase inhibitors and the antiplatelet effects of aspirin. N. Engl. J. Med. 2001, 345: 1809-1817.
Chakraborty I, Das S K, Wang J, Dey S K: Developmental expression of the cyclo-oxygenase-1 and cyclo-oxygenase-2 genes in the peri-implantation mouse uterus and their differential regulation by the blastocyst and ovarian steroids. J. Mol. Endocrinol. 1996, 16: 107-22.
Chandran V R: Amino acid prodrugs. Int. Patent No. WO 2005/046575 A2.
Connor J R, Manning P T: Method for the prevention or treatment of pain, inflammation and inflammation-related disorders with a COX-2 selective inhibitor in combination with a nitric oxide-donating agent and therewith. 2005, US Patent No: 2005/0113409 A1.
Const T, Ferdinandy P: Cardioprotective effects of glyceryl trinitrate: beyond vascular nitrate tolerance. Pharmacol. Ther. 2005, 105: 57-68.
Crankshaw D L, Berkeley L I, Cohen J F, Shirota F N, Nagasawa H T: Double-prodrugs of L-cysteine: Differential protection against acetaminophen-induced hepatotoxicity in mice. J. Biochem. Molecular Toxicology. 2002, 16: 235-44.
Cuzzolin L, Conforti A, Adami A: Anti-inflammatory potency and gastrointestinal toxicity of a new compound. NO-Naproxen. Pharmacol. Res. 1995, 31: 61-65.
Damm J, Rau T, Maihofner C, Pahl A, Brune K: Constitutive expression and localization of COX-1 and COX-2 in rabbit iris and ciliary body. Exp. Eye Res. 2001, 72: 611-21.
Davidge S T: Prostaglandin H synthase and vascular function. Circ. Res. 2001, 89: 650-60.
Davies B, Morris T: Physiological Parameters in laboratory animals and man. Pharm. Res. 1993, 10(7): 1093-95.
Davies N M: NO-naproxen vs. naproxen: ulcerogenic, analgesic and anti-inflammatory effects. Aliment. Pharmacol. Ther. 1997, 11: 69-79.
Del Soldato P: Organic nitrate-based compounds for the treatment of vasculopathies. 2002a. Int. Patent No: WO 02/100400 A1.
Del Soldato P: Drugs for the Alzheimer disease. 2002b. Int. Patent No: WO 02/092072 A2.
Del Soldato P: Drugs for the treatment of arthritis. 2003. Int. Patent No: WO 03/084550 A1.
Del Soldato P, Santus G: Oral pharmaceutical forms of liquid drugs having improved bioavailability. 2004a. Int. Patent No: WO 2004/000273 A1.
Del Soldato P, Santus G: Cyclooxygenase-2 inhibitors. 2004b. Int Patent No: WO 2004/0003000 A1.
Del Soldato P, Santus G: Nitrooxyderivatives of cyclooxygenase-2 inhibitors. 2004c. Int. Patent No: WO 2004/000781 A2.
Dhawan V, Schwalb D J, Shumway M J, Warren M C, Wexler R S, Zemtseva I S, Zifcak B M, Janero D R: Selective nitros(yl)ation induced in vivo by a nitric oxide-donating cyclooxygenase-2 inhibitor: A NObonomic analysis Free Radical. Biology & Medicine. 2005, 39: 1191-1207.
Earl R A, Ezawa M, Fang X, Garvey D S, Gaston R D, Khanapure S P, Letts G L, Lin C-E, Ranatunge R R; Nitrosated nonsteroidal anti-inflammatory compounds, compositions and methods of use. Int. Patent No. W) 2004/004648 A2.
Engelhardt F C, Shi Y J, Cowden C J, Conlon D A, Pipik B, Zhou G, McNamara J M, Dolling U H: Synthesis of a NO-releasing prodrug of rofecoxib. J. Org. Chem. 2006, 71: 480-491.
Fagerholm U, Breuer O, Swedmark S, Hoogstraate J: Pre-clinical pharmacokinetics of the cyclooxygenase-inhibiting nitric oxide donor (CINOD) AZD3582. J. Pharm. Pharmacol. 2005, 57(5): 587-97.
Fagerholm U, Bjornsson M A: Clinical pharmacokinetics of the cyclooxygenase inhibiting nitric oxide donator (CINOD) AZD3582. J. Pharm. Pharmacol. 2005, 57(12):1539-1554.
FitzGerald G A: Parsing an enigma: the pharmacodynamics of aspirin resistance. Lancet. 2003, 361(9357):542-4.
FitzGerald G A: COX-2 and beyond: Approaches to prostaglandin inhibition in human disease. Nat. Rev. Drug Discov. 2003a, 2:879-90.
Frehm E J, Bonaventura J, Gow A J: S-Nitrosohemoglobin: an allosteric mediator of NO group function in mammalian vasculature. Free Radic. Biol. Med. 2004, 37:442-53.
Fung H-L: Biochemical mechanism of nitroglycerin action and tolerance: is this old mystery solved? Annu. Rev. Pharmacol. Toxicol. 2004, 44: 67-85.
Gao J, Kashfi K, Rigas B: In vitro metabolism of nitric oxide-donating aspirin: The effect of positional isomerism. J. Pharmacol. Exp. Ther. 2005, 312: 989-997.
Ghose A K, Viswanadhan V N, Wendoloski J J: Prediction of Hydrophobic (lipophilic) Properties of Small Organic Molecules Using Fragmental Methods: An Analysis of ALOGP and CLOGP Methods. J. Phys. Chem. 1998, A 102: 3762-72.
Gilmer J F, Moriarty L M, Lally M N, Clancy J M: Isosorbide-based aspirin prodrugs II. Hydrolysis kinetics of isosorbide diaspirinate. Eur. J. Pharm. Sci. 2002, 16: 297-304.
Gow A J: Nitric oxide, hemoglobin, and hypoxic vasodilation. Am J. Respir. Cell Mol. Biol. 2005, 32: 479-82.
Hansen J, Mark N, Bundgaard H: Phenyl carbamates of amino acids as prodrug forms for protecting phenols against first-pass metabolism. Int. J. Pharm. 1992, 81: 253-61.
Hanson J, Rolin S, Reynaud D, Qiao N, Kelley L P, Reid H M, Valentin F, Tippins J, Kinsella B T, Masereel B, Pace-Asciak C, Pirotte B, Dogne J M: In vitro and in vivo pharmacological characterization of BM-613 [N-n-pentyl-N′-[2-(4′-methylphenylamino)-5-nitrobenzenesulfonyl]urea], a novel dual thromboxane synthase inhibitor and thromboxane receptor antagonist. J Pharmacol Exp Ther. 2005, 313(1):293-301.
Hinz B, Brune K: Cyclooxygenase-2-10 years later. J. Pharmacol. Exp. Ther. 2002, 300: 367-75.
Hinz B, Brune K, Pahl A: Cyclooxygenase-2 expression in lipopolysaccharide-stimulated human monocytes is modulated by cyclic AMP, prostaglandin E(2), and nonsteroidal anti-inflammatory drugs. Biochem. Biophys. Res. Commun. 2000, 278: 790-96.
Hollander D: Gastrointestinal complications of nonsteroidal anti-inflammatory drugs: prophylactic and therapeutic strategies. Am. J. Med. 1994, 96: 274-281.
Iseki S: Immunocytochemical localization of cyclooxygenase-1 and cyclooxygenase-2 in the rat stomach. Histochem. J. 1995, 27:323-8.
Kartasasmita R E, Laufer S, Lehmann J: NO-Donors (VII): Synthesis and cyclooxygenase inhibitory properties of N- and S-nitrooxypivaloyl-cysteine derivatives of naproxen—A novel type of NO-NSAID. Arch. Pharm. Pharm. Med. Chem. 2002, 8: 363-66.
Keefer L K: Progress toward the clinical application of the nitric oxide-releasing diazeniumdiolates. Annu. Rev. Pharmacol. Toxicol. 2003, 43: 585-607.
Keefer L K: Nitric oxide (NO)- and nitroxyl (HNO)-generating diazeniumdiolates (NONOates): Emerging commercial opportunities. Curr. Topics Med. Chem. 2005, 5: 625-36.
Kitagawa H, Takeda F, Kohei H: Effect of endothelium-derived relaxing factor on the gastric lesion induced by HCl in rats. J. Pharmacol. Exp. Ther. 1990, 253: 1133-1137.
Khanapure S P, Garvey D S, Earl R A, Ezawa M, Fang X, Gaston R D: Substituted aryl compounds as novel cyclooxygenase-2 selective inhibitors, compounds and methods of use. Int. Patent No. WO 02/060378 A2.
Khanapure S P, Garvey D S, Earl R A, Ezawa M, Fang X, Gaston R D: Substituted aryl compounds as novel cyclooxygenase-2 selective inhibitors, compositions and methods of use. 2004, U.S. Pat. No. 6,825,185.
Knaus E E, et al. Novel NSAIDs Possessing a Nitric Oxide Donor Diazen-1-ium-1,2-diolate Moiety: Design, Synthesis, Biological Evaluation and Nitric Oxide Release Studies 2005, USPTO Provisional Patent Application.
Krötz F, Schiele T M, Klauss V, Sohn H Y: Selective COX-2 inhibitors and risk of myocardial infarction. J. Vasc. Res. 2005, 42(4):312-24.
Letts L G, Garvey D S: Nitrosated and/or nitrosylated cyclooxygenase-2 selective inhibitors, compositions and methods of use. Int. Patent No. WO 03/103602 A2.
Lipsky P E: Role of cyclooxygenase-1 and -2 in health and disease. Am. J. Orthop. 1999, 28: 8-12.
Luo C, He M L, Bohlin L: Is COX-2 a perpetrator or a protector? Selective COX-2 inhibitors remain controversial. Acta Pharmacologica Sinica 2005, 26: 926.
Martignoni M, Groothuis G, de Kanter R: Comparison of mouse and rat cytochrome P450-mediated metabolism in liver and intestine. Drug. Metab. Disp. 2006, 34:1047-54.
Mattioni B E, Kauffman G W, Jurs P C, Custer L L, Durham S K, Pearl G M: Predicting the genotoxicity of secondary and aromatic amines using data subsetting to generate a model ensemble. J. Chem. Inf. Comput. Sci. 2003, 43: 949-63.
Mizen, L, Burton, G: The use of esters as prodrugs for oral delivery of beta-lactam antibiotics. 1998, Pharm. Biotech. 11: 345-365.
MacNaughton W K, Cirino G, Wallace J: Endothelium-derived relaxing factor (nitric oxide) has protective actions in the stomach. Life Sci. 1989, 45: 1869-1876.
Mollace V, Muscoli C, Masini E, Cuzzocrea S, Salvemini D: Modulation of prostaglandin biosynthesis by nitric oxide and nitric oxide donors. Pharmacol. Rev. 2005, 57:217-52.
Morrison B W, Christensen S, Yuan W, Brown J, Amlani S, Seidenberg B: Analgesic efficacy of the cyclooxygenase-2-specific inhibitor rofecoxib in post-dental surgery pain: a randomized, control trial. Clin. Ther. 1999, 21: 943-53.
Myers R C, Ballantyne B: Comparative acute toxicity and primary irritancy of various classes of amines. Toxic Subst. Mech. 1997, 16: 151-93.
Nantel F, Meadows E, Denis D, Connolly B. Metters K M, Giaid A: Immunolocalization of cyclooxygenase-2 in the macula densa of human elderly. FEBS Lett. 1999, 457:475-7.
Phelan M J, Bodor N: Improved Delivery Through Biological Membranes. XXXVII. Synthesis and Stability of Novel Redox Derivatives of Naproxen and Indomethacin. Pharm. Res. 1989, 6: 667-76.
Pitman I H: Med. Res. Rev. 1981, 1: 189-214.
Porter G A, Norton T L, Legg V: Using urinary biomarkers to evaluate renal effects of a Cox-2 NSAID in volunteers. Renal Failure 1999, 21(3&4): 311-7.
Poulin P, Thiel F-P: Prediction of Pharmacokinetics Prior to In Vivo Studies. 1. Mechanism-Based Prediction of Volume of Distribution. J. Pharm. Sci. 2002, 91(1): 129-156.
Rainsford K D: Profile and mechanisms of gastrointestinal and other side effects of nonsteroidal anti-inflammatory drugs (NSAIDs). Am. J. Med. 1999, 107: 27S-36S.
Ranatunge R R, Augustyniak M E, Bandarage U K, Earl R A, Ellis J L, Garvey D S, Janero D R, Letts L G, Martino A M, Murty M G, Richardson S K, Schroeder J D, Shumway M J, Tam S W, Trocha A M, Young D V: Synthesis and Selective Cyclooxygenase-2 Inhibitory Activity of a Series of Novel, Nitric Oxide Donor-Containing Pyrazoles. J. Med. Chem. 2004, 47: 2180-93.
Ranatunge R R, Augustyniak M E, Dhawan V, Ellis J L, Garvey D S, Janero D R, Letts G, Richardson S K, Shumway M J, Trocha M, Young D V, Zemtseva I S: Synthesis and anti-inflammatory activity of a series of N-substituted naproxen glycolamides: Nitric oxide-donor naproxen prodrugs. Bioorg. Med. Chem. 2006, 14: 2589-99.
Reicin A, Brown J, Jove M, deAndrade J R, Bourne M, Krupa D, Walters D, Seidenberg B: Efficacy of single-dose and multidose rofecoxib in the treatment of post-orthopedic surgery pain. Am. J. Orthop. 2001, 30: 40-8.
Reuter B, Cirino G, Wallace, J: Markedly reduced intestinal toxicity of a diclofenac derivative. Life Sci. 1994, 55: PL1-PL8.
Rivolta R, Roberto A: New process for the preparation of nitrooxy derivatives of paracetamol. 2005. Int. Patent No. WO 2005/054175 A2.
Rodriguez L A: The effect of NSAIDs on the risk of coronary heart disease: fusion of clinical pharmacology and pharmacoepidemiologic data. Clin. Exp. Rheumatol. 2001, 19: (Suppl 25), S41-S44.
Runkle R, Chaplin M, Boost G, Segre E, Forchielli E: Absorption, Distribution, Metabolism, and Excretion of Naproxen in Various Laboratory Animals and Human Subjects. J. Pharm. Sci. 1972, 61(5): 703-8.
Saavedra J E, Booth M N, Hrabie J A, Davies K M, Keefer L K: piperazine as a linker for incorporating the nitric oxide-releasing diazeniumdiolate group into other biomedically relevant functional molecules. J. Org. Chem. 1999, 64: 5124-31.
Saavedra J E, Shami P J, Wang L Y, Davies K M, Booth M N, Citro M L, Keefer L K: Esterase-sensitive nitric oxide donors of the diazeniumdiolate family: In vitro antileukemic activity. J. Med. Chem. 2000, 43: 261-9.
Satyam A: Prodrugs containing novel bio-cleavable linkers. US Patent No. US 2006/0046967 A1.
Schoen R T, Vender R J: Mechanisms of nonsteroidal anti-inflammatory drugs gastric damage. Am. J. Med. 1989, 86: 449-457.
Showalter B M, Reynolds M M, Valdez C A, Saavedra J E, Davies K M, Klose J R, Churny G N, Citro M L, Barchi Jr. J J, Merz S I, Meyerhoff M E, Keefer L K: Diazeniumdiolate ions as leaving groups in anomeric displacement reactions: A protection-deprotection strategy for ionic diazeniumdiolates. J. Am. Chem. Soc. 2005, 127: 14188-14189.
Silverstein F E, Faich G, Goldstein J L, Simon L S, Pincus T, Whelton A, Makuch R, Eisen G, Agrawal N M, Stenson W F, Burr A M, Zhao W W, Kent J D, Lefkowith J B, Verburg K M, Geis G S: Gastrointestinal toxicity with celecoxib vs. nonsteroidal anti-inflammatory drugs for osteoarthritis and rheumatoid arthritis: the CLASS study: A randomized controlled trial. Celecoxib Long-term Arthritis Safety Study. JAMA. 2000, 284(10):1247-55.
Singel D J, Stamler J S: Chemical physiology of blood flow regulation by red blood cells. Annu. Rev. Physiol. 2005, 67:99-145.
Slater D M, Dennes W J, Campa J S, Poston L, Bennett P R: Expression of cyclooxygenase types-1 and -2 in human myometrium throughout pregnancy. Mol. Hum. Reprod. 1999, 5(9):880-4.
Slater D, Dennes W, Sawdy R, Allport V, Bennett P: Expression of cyclo-oxygenase types-1 and -2 in human fetal membranes throughout pregnancy. J. Mol. Endocrinol. 1999, 22(2):125-30.
Tam D, Tirona R G, Pang S K: Segmental Intestinal Transporters and Metabolic Enzymes on Intestinal Drug Absorption, Drug Metabolism and Disposition, 2003, 31(4): 373-83.
Tang X, Xian M, Trikha M, Honn K V, Wang P G: Synthesis of peptide-diazeniumdiolate conjugates: towards enzyme activated antitumor agents. Tetrahedron Letts. 2001, 42(14): 2625-2629.
Tegeder I, Neupert W, Guhring H, Geisslinger G: Effects of selective and unselective cyclooxygenase inhibitors on prostanoid release from various rat organs. J. Pharmacol. Exp. Ther. 2000, 292(3):1161-8.
Thulesen J, Hartmann B, Nielsen C, Hoist J J, Poulsen S S: Diabetic intestinal growth adaptation and glucagon-like peptide 2 in the rat: effects of dietary fibre. Gut 1999, 45:672-8.
Tong V, Abbott F S, Mbofana S, Walker M J A: In Vitro Investigation of the Hepatic Extraction of RSD1070, A Novel Antiarrhythmic Compound, J. Pharm. Pharmaceut. Sci. 2001, 4(1):15-23.
Tubaro E, Belogi L, Mezzadri C M: Anti-inflammatory and antiplatelet effects of amtolmetin guacyl, a new gastroprotective non-steroidal anti-inflammatory drug. Arzneimittelforschung. 2001, 51(9):737-42.
Turkan H, Beyan C, Karabiyik L, Guner D, Kaptan K: The effects of desflurane on human platelet aggregation in vitro. Int J Hematol. 2004, 80(1):91-3.
Vane J, Botting R M: Mechanism of action of nonsteroidal anti-inflammatory drugs. Am. J. Med. 1998, 104: (Suppl 3A), 2S-8S.
Velázquez C, Praveen Rao P N, Knaus E E: J. Novel nonsteroidal anti-inflammatory drugs possessing a nitric oxide donor diazen-1-ium-1,2-diolate moiety: Design, synthesis, biological evaluation and nitric oxide release studies. J. Med. Chem. 2005, 48:4061-4067.
Wallace J, Reuter B, Cicala C, McKnight W, Grisham M, Cirino G: Novel nonsteroidal anti-inflammatory drug derivatives with markedly reduced ulcerogenic properties in the rat. Gastroenterology 1994a, 107: 173-179.
Wallace J, Reuter B, Cicala C, McKnight W, Grisham M, Cirino, G: A Diclofenac derivative without ulcerogenic properties. Eur. J. Pharmacol. 1994b, 257: 249-255.
Wallace J L, Soldato P D: The therapeutic potential of NO-NSAIDs. Fund. Clin. Pharmacol. 2003, 17: 11-20.
Warner T D, Mitchell J A: Cyclooxygenases: new forms, new inhibitors, and lessons from the clinic. FASEB J. 2004, 18(7):790-804.
Yu R J, Van Scott E J: Systemic administration of therapeutic amino acids and N-acetylamino acids. US 2006/0063827.
Zafirovska K G, Bogdanovska S V, Marina N, Gruev T, Lozance L: Urinary excretion of three specific renal tubular enzymes in patients treated with nonsteroidal anti-inflammatory drugs (NSAID). Ren. Fail. 1993, 15(1): 51-4.
Zimmermann K C, Sarbia M, Schror K, Weber A A: Constitutive cyclooxygenase-2 expression in healthy human and rabbit gastric mucosa. Mol. Pharmacol. 1998, 54(3):536-40.
de Zwart L L, Rompelberg C J M, Sips A J A M, Welink J, van Engelen J G M: Rijksinstituut Voor Volksgezondheid En Milieu RIVM report 623860 010: Anatomical and physiological differences between various species used in studies on the pharmacokinetics and toxicology of xenobiotics. A review of literature. 1999, 1-100.

1-35. (canceled)

36. A method of pairing a therapeutic agent with an appropriate nitric oxide donor to create an effective prodrug molecule, comprising:
(i) obtaining in vitro or in silico pharmacokinetic and/or pharmacodynamic data of the therapeutic agent, nitric oxide donor and nitric oxide from said donor;
(ii) placing the data into a physiologically-based pharmacokinetic/pharmacodynamic model comprising:
(a) a first compartment model which divides a gastrointestinal tract into compartments, wherein said compartment model describes gastrointestinal absorption of said prodrug molecule; and
(b) a second compartment model which divides a body into plasma/blood and tissue compartments, wherein said compartment model describes the time course of the therapeutic agent, the nitric oxide donor, and nitric oxide in gastrointestinal tract, blood, and tissues; and
(iii) generating output parameters from said pharmacokinetic model, wherein said output parameters determine the appropriateness of pairing of said therapeutic agent with said nitric oxide donor to create an effective prodrug molecule.

37. The method of claim 1, wherein the first compartment model divides a gastrointestinal tract into seven compartments, wherein the tissue compartments are selected from the group consisting of heart, liver, and kidney, and wherein the therapeutic agent is selected from the group consisting of nonsteroidal anti-inflammatory drugs and antibiotics.

38. The method of claim 1, wherein the in vitro or in silico data are selected from the group consisting of pKa values, octanol/water partition coefficients, solubility data, log P values, permeability values, metabolism data, hydrolysis data, serum protein binding data, nitric oxide release rate, pharmacokinetic and pharmacodynamic data of a prodrug and a therapeutic agent, and stability data in gastric and intestinal environments.

39. The method of claim 1, wherein the in vitro or in silico data comprise volumes of distribution for the prodrug and the nitric oxide donor.

40. A prodrug molecule selected by the method of claim 1, wherein said prodrug molecule can be enzymatically or hydrolytically cleaved to give a therapeutic agent and a nitric oxide donor.

41. The prodrug molecule of claim 5, wherein the therapeutic agent is a nonsteroidal anti-inflammatory drug or an antibiotic.

42. The prodrug molecule of claim 5, wherein the nitric oxide donor is diazen-1-ium-1,2-diolate.

43. The prodrug molecule of claim 6, wherein the nonsteroidal anti-inflammatory drug is a non-selective cyclooxygenase isozyme inhibitor or a cyclooxygenase-2 inhibitor.

44. The prodrug molecule of claim 8, wherein the non-selective cyclooxygenase isozyme inhibitor is selected from the group consisting of acetylsalicylic acid (CH₃COOC₆H₄COOH), IBUPROFEN (C₁₃H₁₈O₂), NAPROXEN (C₁₄H₁₄O₃,), indomethacin (C₁₉H₁₆ClNO₄), and diclofenac (Cl₁₄H₁₀Cl₂NNaO); or the cyclooxygenase-2 inhibitor comprises a carboxyl group.

45. The prodrug molecule of claim 5, wherein said donor has a half-life that is longer than the total time period for hydrolysis and absorption, wherein a therapeutic dosage of nitric oxide is released into enterocytes, thereby protecting them against damage caused by gastrointestinal irritation, bleeding or ulceration, or wherein a therapeutic dosage of nitric oxide is released into the blood stream, thereby protecting one or more organ systems, wherein the organ systems are selected from the group consisting of heart, kidney, and cardiovascular system.

46. The prodrug molecule of claim 5 comprising:
(i) a nitric oxide donor linked to an amino acid through a linkage that is susceptible to enzymatic hydrolysis or cleavage; and
(ii) a therapeutic agent directly linked to said amino acid, or linked to said amino acid through a spacer, wherein the linkage between the therapeutic agent and the spacer, or the linkage between the spacer and the amino acid is susceptible to enzymatic hydrolysis or cleavage,
wherein release of the nitric oxide donor and the therapeutic agent from the prodrug molecule can be controlled independently.

47. The prodrug molecule of claim 11, wherein the amino acid is selected from the group consisting of hydroxyproline, glutamic acid, and aspartic acid, and wherein the linkage susceptible to enzymatic hydrolysis or cleavage is selected from the group consisting of ester linkage, thioester linkage, amide linkage, and sulfonamide linkage.

48. The prodrug molecule of claim 11, wherein said therapeutic agent is a nonsteroidal anti-inflammatory drug or an antibiotic, and said donor is diazen-1-ium-1,2-diolate.

49. The prodrug molecule of claim 5 with the formula of: 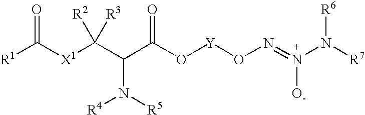
wherein R¹ is the uncarboxylated core of a non-steroidal anti-inflammatory drug, or a structure of formula II: 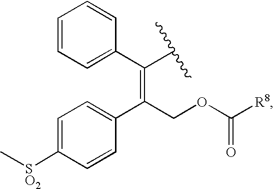
wherein R⁸ is hydrogen, an unsubstituted or substituted C₁₋₁₂ straight chain alkyl, or an unsubstituted or substituted C₃₋₁₂ branched chain alkyl;
wherein X¹ has a formula selected from the group consisting of:
(i) formula III: 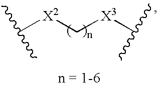
wherein X² is oxygen, sulfur, or NH, and X³ is oxygen, sulfur, or NH,
(ii) formula IV: 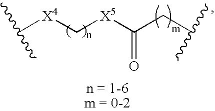
wherein X⁴ is oxygen, sulfur, or NH, and X⁵ is oxygen, sulfur, or NH,
(iii) formula V: 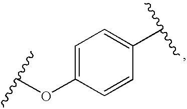
and
(iv) formula VI: 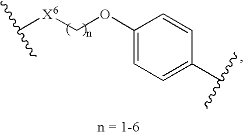
wherein X⁶ is oxygen, sulfur, or NH;
wherein R² is hydrogen, an unsubstituted or substituted C₁₋₁₂ straight chain alkyl, or an unsubstituted or substituted C₃₋₁₂ branched chain alkyl;
wherein R³ is hydrogen, an unsubstituted or substituted C₁₋₁₂ straight chain alkyl, or an unsubstituted or substituted C₃₋₁₂ branched chain alkyl;
wherein R⁴ is selected from the group consisting of:
(i) hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl,
(ii) formula VII: 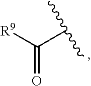
wherein R⁹ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, an amide derivative linked via a carboxy group of an amino acid, or an amide derivative of a polypeptide,
(iii) formula VIII: 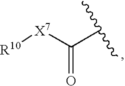
wherein X⁷ is oxygen, sulfur, or NH, and R¹⁰ is an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl, and
(iv) formula IX: 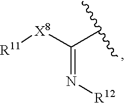
wherein X⁸ is oxygen, sulfur, or NH; and R¹¹ is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl; and R¹² is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl;
wherein R⁵ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, a structure of formula VIII, or a structure of formula IX;
wherein R⁶ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII;
wherein R⁷ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII, or a structure of formula XI: 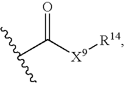
wherein X⁹ is oxygen, sulfur, or NH, and R¹⁴ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, or an amino acid wherein X⁹ is the amino group of the amino acid; and
wherein Y is a structure of the formula XIII: 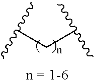
or a structure of the formula XIV: 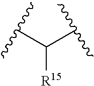
wherein R¹⁵ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl.

50. The prodrug molecule of claim 14, wherein NR⁶R⁷ is a heterocycle of
(i) formula X: 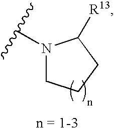
wherein R¹³ is hydrogen, or
(ii) formula XII: 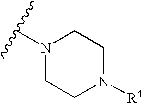
wherein R⁴ is selected from the group consisting of:
(i) hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl,
(ii) formula VII: 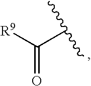
wherein R⁹ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, an amide derivative linked via a carboxy group of an amino acid, or an amide derivative of a polypeptide,
(iii) formula VIII: 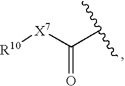
wherein X⁷ is oxygen, sulfur, or NH, and R¹⁰ is an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl, and
(iv) formula IX: 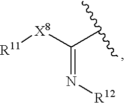
wherein X⁸ is oxygen, sulfur, or NH; and R¹¹ is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl; and R¹² is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl.

51. The prodrug molecule of claim 5 with the formula of 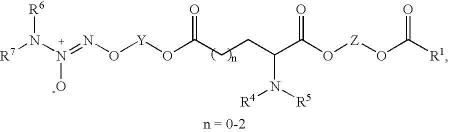
wherein Z is a structure of the formula XIII, 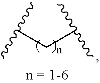
or a structure of the formula XIV, 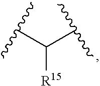
wherein R¹⁵ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl;
wherein R¹ is the uncarboxylated core of a non-steroidal anti-inflammatory drug, or a structure of formula II: 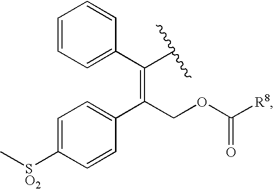
wherein R⁸ is hydrogen, an unsubstituted or substituted C₁₋₁₂ straight chain alkyl, or an unsubstituted or substituted C₃₋₁₂ branched chain alkyl;
wherein R⁴ is selected from the group consisting of:
(i) hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl,
(ii) formula VII: 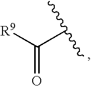
wherein R⁹ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, an amide derivative linked via a carboxy group of an amino acid, or an amide derivative of a polypeptide,
(iii) formula VIII: 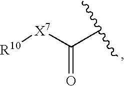
wherein X⁷ is oxygen, sulfur, or NH, and R¹⁰ is an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl, and
(iv) formula IX: 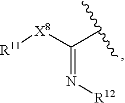
wherein X⁸ is oxygen, sulfur, or NH; and R¹¹ is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl; and R¹² is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl;
wherein R⁵ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, a structure of formula VIII, or a structure of formula IX;
wherein R⁶ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII;
wherein R⁷ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII, or a structure of formula XI: 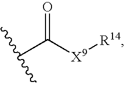
wherein X⁹ is oxygen, sulfur, or NH, and R¹⁴ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, or an amino acid wherein X⁹ is the amino group of the amino acid; and
wherein Y is a structure of the formula XIII: 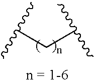
or a structure of the formula XIV: 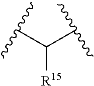
wherein R¹⁵ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl (an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl.

52. The prodrug molecule of claim 5 with the formula of 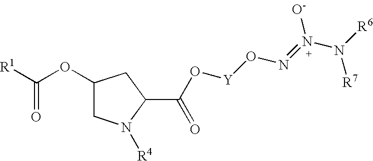
wherein R¹ is the uncarboxylated core of a non-steroidal anti-inflammatory drug, or a structure of formula II: 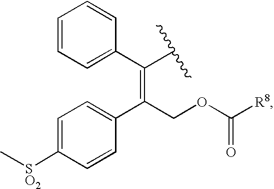
wherein R⁸ is hydrogen, an unsubstituted or substituted C₁₋₁₂ straight chain alkyl, or an unsubstituted or substituted C₃₋₁₂ branched chain alkyl;
wherein R⁴ is selected from the group consisting of:
(i) hydrogen, an unsubstituted or substituted C₁₋₁₂ straight chain alkyl an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl,
(ii) formula VII: 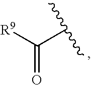
wherein R⁹ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, an amide derivative linked via a carboxy group of an amino acid, or an amide derivative of a polypeptide,
(iii) formula VIII: 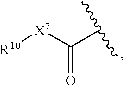
wherein X⁷ is oxygen, sulfur, or NH, and R¹⁰ is an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl, and
(iv) formula IX: 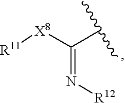
wherein X⁸ is oxygen, sulfur, or NH; and R¹¹ is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl; and R¹² is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl;
wherein R⁶ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII;
wherein R⁷ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII, or a structure of formula XI: 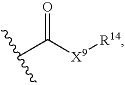
wherein X⁹ is oxygen, sulfur, or NH, and R¹⁴ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, or an amino acid wherein X⁹ is the amino group of the amino acid; and
wherein Y is a structure of the formula XIII: 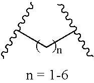
or a structure of the formula XIV: 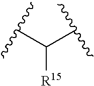
wherein R¹⁵ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl.

53. The prodrug molecule of claim 5 with the formula of 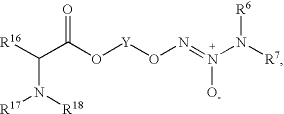
wherein R¹⁸ is selected from the group consisting of:
(i) formula XVIII: 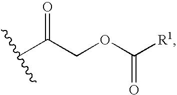
(ii) formula XIX: 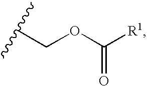
(iii) formula XX: 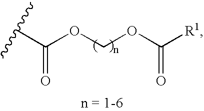
and
(iv) formula XXI: 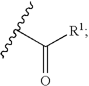
wherein the substructure: 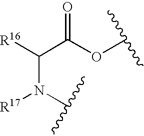
represents the core structure of an amino acid selected from alanine, 2-aminobutyric acid, acid, α-aminosuberic acid, arginine, asparagines, aspartic acid, citrulline, β-cyclohexylalanine, cysteine, 3,4-dehydroproline, glutamic acid, glutamine, glycine, histadine, homocitrulline, homoserine, hydroxyproline, β-hydroxyvaline, isoleucine, leucine, lysine, methionine, norleucine, novaline, ornithine, penicillamine, phenylalanine, phenylglycine, proline, pyroglutamine, sarcosine, serine, threonine, tryptophan, tyrosine and valine;
wherein R¹ is the uncarboxylated core of a non-steroidal anti-inflammatory drug, or a structure of formula II: 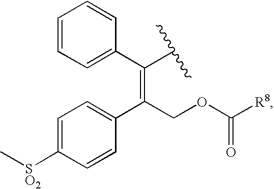
wherein R⁸ is hydrogen, an unsubstituted or substituted C₁₋₁₂ straight chain alkyl, or an unsubstituted or substituted C₃₋₁₂ branched chain alkyl;
wherein R⁶ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII;
wherein R⁷ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII, or a structure of formula XI: 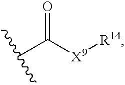
wherein X⁹ is oxygen, sulfur, or NH, and R¹⁴ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, or an amino acid wherein X⁹ is the amino group of the amino acid; and
wherein Y is a structure of the formula XIII: 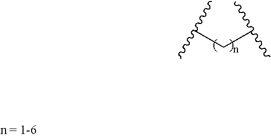
or a structure of the formula XIV: 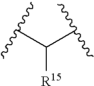
wherein R¹⁵ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl.

54. The prodrug molecule of claim 5 with the formula of 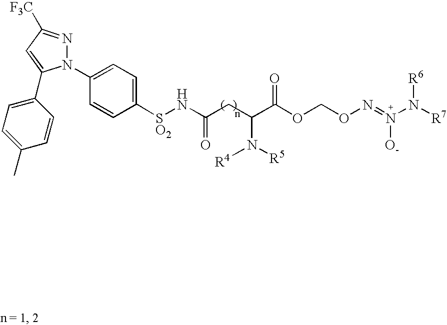
wherein R⁴ is selected from the group consisting of:
(i) hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl,
(ii) formula VII: 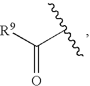
wherein R⁹ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, an amide derivative linked via a carboxy group of an amino acid, or an amide derivative of a polypeptide,
(iii) formula VIII: 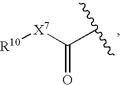
wherein X⁷ is oxygen, sulfur, or NH, and R¹⁰ is an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl, and
(iv) formula IX: 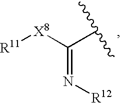
wherein X⁸ is oxygen, sulfur, or NH; and R¹¹ is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl; and R¹² is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl;
wherein R⁵ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, a structure of formula VIII, or a structure of formula IX;
wherein R⁶ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII; and
wherein R⁷ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII, or a structure of formula XI: 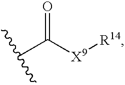
wherein X⁹ is oxygen, sulfur, or NH, and R¹⁴ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, or an amino acid wherein X⁹ is the amino group of the amino acid.

55. The prodrug molecule of claim 5 with the formula of 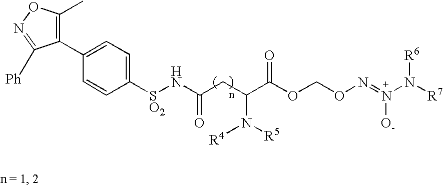
wherein R⁴ is selected from the group consisting of:
(i) hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl,
(ii) formula VII: 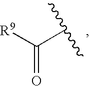
wherein R⁹ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, an amide derivative linked via a carboxy group of an amino acid, or an amide derivative of a polypeptide,
(iii) formula VIII: 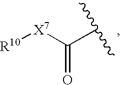
wherein X⁷ is oxygen, sulfur, or NH, and R¹⁰ is an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl, and
(iv) formula IX: 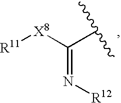
wherein X⁸ is oxygen, sulfur, or NH; and R¹¹ is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl; and R¹² is a hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, or an unsubstituted or substituted heteroaryl;
wherein R⁵ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, a structure of formula VIII, or a structure of formula IX;
wherein R⁶ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII; and
wherein R⁷ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, a structure of formula VII, or a structure of formula VIII, or a structure of formula XI: 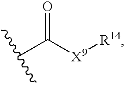
wherein X⁹ is oxygen, sulfur, or NH, and R¹⁴ is hydrogen, an unsubstituted or substituted C₁₋₁₂ straight chain alkyl, an unsubstituted or substituted C₃₋₁₂ branched chain alkyl, an unsubstituted or substituted C₁₋₁₂ straight chain alkenyl, an unsubstituted or substituted C₃₋₁₂ branched chain alkenyl, an unsubstituted or substituted benzyl, an unsubstituted or substituted phenyl, an unsubstituted or substituted C₁₋₄ aryl alkyl, an unsubstituted or substituted heteroaryl, or an amino acid wherein X⁹ is the amino group of the amino acid.
